SEQUENCE LISTING (1 sequence)

SEQ ID NO: 1 - protein, 38 aa, Artificial Sequence
  Synthetic
RQIKIWFQNRRMKWKKMGQVGRQLAIIGDDINRRYDSC

(19) United States
(12) Patent Application Publication
(10) Pub. No.: US 2011/0281934 A1
(43) Pub. Date: Nov. 17, 2011

(54) MICELLES OF HYDROPHILICALLY SHIELDED MEMBRANE-DESTABILIZING COPOLYMERS

(75) Inventors: Paul Johnson, Snohomish, WA (US); Patrick S. Stayton, Seattle, WA (US); Allan S. Hoffman, Seattle, WA (US); Robert Overell, Shoreline, WA (US); Anna Gall, Woodinville, WA (US); Mary Prieve, Lake Forest Park, WA (US); Amber Paschal, Redmond, WA (US); Charbel Diab, Seattle, WA (US); Priyadarsi De, Mohanpur, West Bengal (IN)

(73) Assignees: PHASERX, INC., Seattle, WA (US); UNIVERSITY OF WASHINGTON, Seattle, WA (US)

(21) Appl. No.: 13/127,962

(22) PCT Filed: May 13, 2009
(86) PCT No.: PCT/US2009/043860

§ 371 Date: Jul. 26, 2011

Related U.S. Application Data
(60) Provisional application No. 61/112,054, filed on Nov. 6, 2008.
(60) Provisional application No. 61/112,048, filed on Nov. 6, 2008.
(60) Provisional application No. 61/140,779, filed on Dec. 24, 2008.
(60) Provisional application No. 61/140,774, filed on Dec. 24, 2008.
(60) Provisional application No. 61/171,369, filed on Apr. 21, 2009.
(60) Provisional application No. 61/171,358, filed on Apr. 21, 2009.

(51) Int. Cl. A61K 31/713; C08F 271/02; C08F 265/04

(52) U.S. Cl. 514/44 A; 525/54.2; 525/294; 525/280

(57) ABSTRACT

Provided herein are micelles comprising a plurality of copolymers. In certain instances, micelles provided herein are pH sensitive particles.

### CROSS-REFERENCE

This application claims the benefit of U.S. Provisional Application No. 61/112,048, filed Nov. 6, 2008, U.S. Provisional Application No. 61/140,774, filed Dec. 24, 2008, and U.S. Provisional Application No. 61/171,369, filed Apr. 21, 2009, U.S. Provisional Application No. 61/140,779 filed Dec. 24, 2008, U.S. Provisional Application No. 61/112,054 filed Nov. 6, 2008, U.S. Provisional Application No. 61/171,358 filed Apr. 21, 2009, each of which is hereby incorporated by reference in its entirety.

### FIELD OF THE INVENTION

Described herein are hydrophilically shielded micelles formed from polymers and the use of such micelles.

### BACKGROUND OF THE INVENTION

In certain instances, it is beneficial to provide therapeutic agents (e.g., oligonucleotides) to living cells. In some instances, delivery of polynucleotides to a living cell provides a therapeutic benefit.

### SUMMARY OF THE INVENTION

Provided herein are micelles that are composed of a plurality of hydrophilically-shielded membrane-destabilizing block copolymers. That is, the block copolymers that comprise the micelle comprise both a hydrophilic shielding portion and a pH-dependent membrane-destabilizing portion. The block copolymers optionally include further portions, but at the least the block copolymers have both of the aforementioned portions. The hydrophilic shielding portion of the block copolymer is comprised of monomeric units with a hydrophilic pendant group, including with a polyoxylated alkyl pendant group, and the pH dependent membrane destabilizing portion is a hydrophobic copolymer block that comprises a first chargeable species that is anionic at about neutral pH. When the pH is at about the pKₐ of the chargeable species, there will exist an equilibrium distribution of chargeable species in both forms. In the case of an anionic species, about 50% of the population will be anionic and about 50% will be non-charged when the pH is at the pKₐ of the anionic species. The further the pH is from the pKₐ of the chargeable species, there will be a corresponding shift in this equilibrium such that at higher pH values, the anionic form will predominate and at lower pH values, the uncharged form will predominate. The embodiments described herein include the form of the copolymers at any pH value.

In some embodiments the micelle further includes a therapeutic agent and the hydrophilic shielding portion enhances the stability of the therapeutic agent (e.g., polynucleotide or peptide, etc.), including shielding the therapeutic agent against enzymatic-based digestion. In some instances, a shielding agent reduces toxicity of micelles described herein (e.g., block copolymer attached to polynucleotides). In certain embodiments, the hydrophilic shielding portion also includes a polynucleotide carrier block/segment, and the hydrophilic shielding serves to shield, at least in part, the charge (e.g., cationic charges) on the polynucleotide carrier block/segment. In some embodiments, the therapeutic agent is not in the core of the micelle.

In some embodiments, the compositions described herein comprise a polymeric micelle (e.g., a micelle that comprises polymers) and a polynucleotide associated with the micelle, the micelle comprising a block copolymer including a hydrophilic block and a hydrophobic block, such that the micelle is stable in an aqueous medium at pH 7.4, the hydrophilic block of the copolymer comprising a plurality of constitutional units derived from a polymerizable monomer having a pendant hydrophilic group. One example of a pendant hydrophilic group is Y₆-Q₆: Y₆ is selected from the group consisting of a covalent bond, (1C-10C)alkyl-, —C(O)O(2C-10C) alkyl-, —OC(O)(1C-10C) alkyl-, —O(2C-10C)alkyl- and —S(2C-10C)alkyl- —C(O)NR₆(2C-10C) alkyl-; Q₆ is a residue selected from the group consisting of residues which are hydrophilic at physiologic pH and are substantially non-charged at physiologic pH (e.g., hydroxy, polyoxylated alkyl, polyethylene glycol, polypropylene glycol, thiol, or the like).

In some embodiments, the compositions described herein comprise a polymeric micelle (e.g., a micelle that comprises polymers) and a polynucleotide associated with the micelle, the micelle comprising a block copolymer including a hydrophilic block and a hydrophobic block, such that the micelle is stable in an aqueous medium at pH 7.4, the hydrophilic block of the copolymer comprising a plurality of constitutional units derived from a polymerizable monomer having a pendant hydrophilic group comprising a moiety of formula I

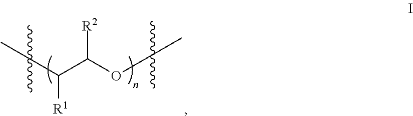

where R¹ and R² are each independently selected from the group consisting of hydrogen, halogen, C₁-C₃ haloalkyl, and optionally substituted C₁-C₃ alkyl, and n is an integer ranging from 2 to 20.

Provided, in some embodiments, is a composition comprising a micelle and a polynucleotide associated with the micelle, the micelle comprising a plurality of block copolymers, each including hydrophilic block and a hydrophobic block;
the micelle being stable in an aqueous medium of about neutral pH;
the hydrophilic block of the copolymer comprising a plurality of constitutional units from a polymerizable monomer having a pendant group comprising a moiety of formula I

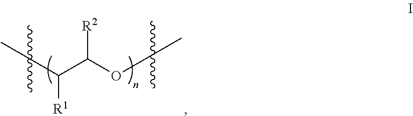

where
R¹ and R² are each independently selected from the group consisting of hydrogen, halogen, C₁-C₃ fluoroalkyl, and optionally substituted C₁-C₃ alkyl, and
n is an integer ranging from 2 to 20.

In some embodiments, the hydrophobic block comprises a pH dependent membrane destabilizing block.

In some embodiments, the pH dependent membrane destabilizing block comprises a plurality of pendant groups that are anionic at about neutral pH, and uncharged at about an endosomal pH.

In some embodiments, the pH dependent membrane destabilizing block comprises a plurality of pendant groups that are cationic at about neutral pH, and cationic at about an endosomal pH.

In some embodiments, the pH dependent membrane destabilizing block further comprises a pendant group that is cationic at about neutral pH and cationic at about an endosomal pH.

In some embodiments, the pH dependent membrane destabilizing block further comprises a pendant group that is hydrophobic at about neutral pH and at about an endosomal pH.

In some embodiments, the polynucleotide is not in the core of the micelle.

In some embodiments, the block copolymer further comprises a plurality of constitutional units having a cationic species in ionic association with the polynucleotide.

In some embodiments, the hydrophilic block of the block copolymer further comprises a plurality of constitutional units having a cationic species in ionic association with the polynucleotide.

In some embodiments, the micelle is covalently coupled to the polynucleotide.

Provided, in some embodiments, is a polymeric micelle, the micelle comprising

a block copolymer comprising a hydrophilic block and a hydrophobic block;
the micelle being stable in an aqueous medium of about neutral pH;
the hydrophilic block of the copolymer comprising a plurality of constitutional units from a polymerizable monomer having a pendant group comprising a moiety of formula I

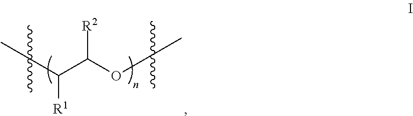

where R¹ and R² are each independently selected from the group consisting of hydrogen, halogen, C₁-C₃ fluoroalkyl, and optionally substituted C₁-C₃ alkyl, and
n is an integer ranging from 2 to 20, and
   an endosomolytic agent.

In some embodiments, the endosomolytic agent is a pH-dependent membrane destabilizing block.

In some embodiments, the block copolymer comprises the pH-dependent membrane disrupting polymer.

In some embodiments, the hydrophobic block of the block copolymer comprises the pH-dependent membrane disrupting polymer.

In some embodiments, the pH dependent membrane destabilizing polymer comprises a plurality of pendant groups that are anionic at about neutral pH, and uncharged at about an endosomal pH.

In some embodiments, the pH dependent membrane destabilizing polymer further comprises a plurality of pendant groups that are cationic at about neutral pH and cationic at about an endosomal pH.

In some embodiments, the pH dependent membrane destabilizing polymer further comprises a plurality of pendant groups that are hydrophobic at about neutral pH and at about an endosomal pH.

In some embodiments, the hydrophilic block of the block copolymer further comprises a plurality of constitutional units having a cationic species in ionic association with the polynucleotide.

In some embodiments, the micelle is covalently coupled to the polynucleotide.

Provided, in some embodiments, is a block copolymer comprising one or more hydrophilic blocks and one or more hydrophobic blocks,
the one or more hydrophilic blocks comprising
   a plurality of constitutional units having a species charged or chargeable to a cation, and
   a plurality of constitutional units from a polymerizable monomer having a pendant group comprising a moiety of formula I

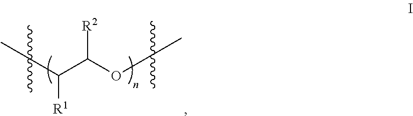

where R¹ and R² are each independently selected from the group consisting of hydrogen, halogen, C₁-C₃ fluoroalkyl, and optionally substituted C₁-C₃ alkyl, and
n is an integer ranging from 2 to 20, and
   the one or more hydrophobic blocks comprising
      a plurality of constitutional units having a species charged or chargeable to an anion, and
      a plurality of constitutional units having a hydrophobic species.

Further provided is a polymeric micelle comprising the block copolymer described above.

In some embodiments, the block copolymer further comprises a plurality of constitutional units having a species charged or chargeable to a cation.

In some embodiments, the hydrophilic block of the block copolymer further comprises a plurality of constitutional units having a species charged or chargeable to a cation.

In some embodiments, the block copolymer further comprises a plurality of constitutional units having a species charged or chargeable to an anion, and a plurality of constitutional units having a hydrophobic species.

In some embodiments, the hydrophobic block of the block copolymer further comprises a plurality of constitutional units having a species charged or chargeable to an anion, and a plurality of constitutional units having a hydrophobic species.

In some embodiments, the hydrophobic block of the block copolymer further comprises a plurality of constitutional units having a species charged or chargeable to an anion, a plurality of constitutional units having a species charged or chargeable to a cation, and a plurality of constitutional units having a hydrophobic species.

In some embodiments, the hydrophobic block of the block copolymer further comprises a plurality of constitutional units having a species charged or chargeable to an anion, a plurality of constitutional units having a species charged or chargeable to a cation, and a plurality of constitutional units having a hydrophobic species, the hydrophobic block having a substantially neutral overall charge in an aqueous medium at pH 7.4.

In some embodiments, the constitutional units are derived from a polymerizable monomer.

In some embodiments, the polymerizable monomer is an ethylenically unsaturated monomer.

In some embodiments, the polymerizable monomer is an acrylic monomer or a vinylic monomer.

In some embodiments, the polymerizable monomer is an acrylic monomer selected from an optionally substituted acrylic acid, an optionally substituted acrylamide, and an optionally substituted acrylate.

In some embodiments, the polymerizable monomer is selected from an optionally C₁-C₃ alkyl-substituted acrylic acid, an optionally C₁-C₃ alkyl-substituted acrylamide, and an optionally C₁-C₃ alkyl-substituted acrylate.

In some embodiments, the polymerizable monomer is a monomer having a formula II

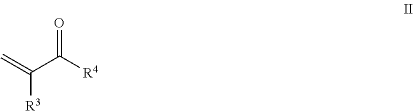

wherein
R³ is hydrogen, halogen, hydroxyl, or optionally substituted C₁-C₃ alkyl;
R⁴ is —SR⁵, —OR⁵, —NR⁶R⁷, or
R⁴ is a polyoxylated alkyl, optionally substituted by hydroxyl, thiol, —NR⁹R¹⁰, a cleavable moiety or a functionalizable moiety;
R⁵ is a polyoxylated alkyl, optionally substituted by hydroxyl, thiol, —NR⁹R¹⁰, a cleavable group or a functionalizable group;
R⁶ and R⁷ are each independently H or polyoxylated alkyl, optionally substituted by hydroxyl, thiol, —NR⁹R¹⁰, a cleavable group or a functionalizable group, provided that R⁶ and R⁷ are not both H; or
R⁶ and R⁷ together with the Nitrogen to which they are attached form an optionally substituted heterocycle;
R⁹ and R¹⁰ are each independently H or C₁-C₆ alkyl; or
R⁹ and R¹⁰ together with the nitrogen to which they are attached form a heterocycle.

In some embodiments, R⁴ is an optionally substituted polyoxylated alkyl.

In some embodiments, the polyoxylated alkyl is selected from an oligosaccharide, a polyethylene glycol group, and a polypropylene glycol group, including optionally substituted groups thereof.

In some embodiments, the block copolymer comprises a plurality of constitutional units derived from a polymerizable monomer having a formula III

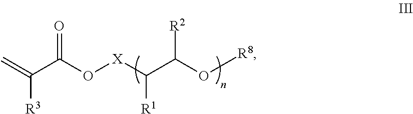

where
X is absent or optionally substituted C₁-C₃ alkyl;
R¹, R² and R³ are each independently hydrogen, halogen, C₁-C₃ fluoroalkyl or optionally substituted C₁-C₃ alkyl;
n is an integer ranging from 2 to 20,
R⁸ is hydrogen, C₁-C₆ alkyl, C₁-C₆ alkenyl, C₁-C₆ alkynyl, cycloalkyl, heterocycloalkyl, aryl, or heteroaryl optionally substituted by hydroxyl, thiol, —NR⁹R¹⁰, a cleavable group or a functionalizable group;
R⁹ and R¹⁰ are each independently H or C₁-C₆ alkyl; or
R⁹ and R¹⁰ together with the nitrogen to which they are attached form a heterocycle.

In some embodiments, R¹ and R² are each H.

In some embodiments, the block copolymer comprises a plurality of constitutional units derived from a polymerizable monomer having a formula IV

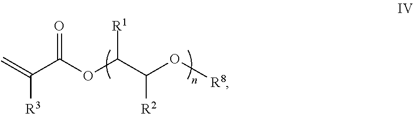

where
R¹, R² and R³ are each independently hydrogen, halogen, C₁-C₃ fluoroalkyl or optionally substituted C₁-C₃ alkyl;
n is an integer ranging from 2 to 20,
R⁸ is hydrogen, C₁-C₆ alkyl, C₁-C₆ alkenyl, C₁-C₆ alkynyl, cycloalkyl, heterocycloalkyl, aryl, or heteroaryl optionally substituted by hydroxyl, thiol, —NR⁹R¹⁰, a cleavable group or a functionalizable group;
R⁹ and R¹⁰ are each independently H or C₁-C₆ alkyl; or
R⁹ and R¹⁰ together with the nitrogen to which they are attached form a heterocycle.

In some embodiments, R¹ and R² are each H.

In some embodiments, the hydrophilic block of the block copolymer is a random copolymer comprising at least about 10% by weight of constitutional units derived from a polymerizable monomer having a pendant group comprising a moiety of Formula I, Formula II, Formula III or Formula IV.

In some embodiments, the hydrophilic block of the block copolymer is a random copolymer comprising at least about 30% by weight of constitutional units derived from a polymerizable monomer having a pendant group comprising a moiety of Formula I, Formula II, Formula III or Formula IV.

In some embodiments, the hydrophilic block of the block copolymer is a random copolymer comprising at least about 50% by weight of constitutional units derived from a polymerizable monomer having a pendant group comprising a moiety of Formula I, Formula II, Formula III or Formula IV.

In some embodiments, the hydrophilic block of the block copolymer is a random copolymer comprising at least about 65% by weight of constitutional units derived from a polymerizable monomer having a pendant group comprising a moiety of Formula I, Formula II, Formula III or Formula IV, and in each case, where n is an integer ranging from 5 to 12.

In certain embodiments, provided herein is a micelle in which at least one block of one or more of the block copolymers is a gradient block.

In some embodiments, provided herein is a hydrophilically-shielded micelle having membrane-destabilizing copolymers that comprises at least one research reagent. In certain embodiments, a hydrophilically-shielded micelle having membrane-destabilizing copolymers described herein comprises at least one diagnostic agent. In some embodiments, the hydrophilically-shielded micelle having membrane-destabilizing copolymers comprises at least one therapeutic agent. In specific embodiments, the therapeutic agent is attached to the hydrophilic block of at least one of the block copolymers in the micelle by a covalent bond, a non-covalent interaction, or a combination thereof. In some embodiments, a hydrophilically-shielded micelle having membrane-destabilizing copolymers provided herein comprises a first therapeutic agent attached to the hydrophilic block of at least one of the block copolymers and at least one second therapeutic agent within the core portion of the micelle. In some embodiments, each hydrophilically-shielded micelle having membrane-destabilizing copolymers comprises on average 1-5, 5-250, 5-1000, 250-1000, at least 2, at least 5, at least 10, at least 20, or at least 50 therapeutic agents. In some embodiments, a therapeutic agent provided in the micelles described herein comprises at least one nucleotide, at least one carbohydrate or at least one amino acid. In certain embodiments, the therapeutic agent is a polynucleotide, an oligonucleotide, a gene expression modulator, a knockdown agent, an siRNA, an RNAi agent, a dicer substrate, an miRNA, an shRNA, an antisense oligonucleotide, or an aptamer. In some embodiments, the therapeutic agent is a proteinaceous therapeutic agent (e.g., a protein, peptide, enzyme, dominant-negative protein, hormone, antibody, antibody-like molecule, or antibody fragment). In certain embodiments, the therapeutic agent is a carbohydrate, or a small molecule with a molecular weight of greater than about 500 Daltons. In some embodiments, one or more of the plurality of block copolymers is attached to a therapeutic agent.

In some embodiments, provided herein is a hydrophilically-shielded micelle having membrane-destabilizing copolymers that comprises at least one targeting moiety.

In certain embodiments, the hydrophilic block of the block copolymers is charged or chargeable. In some embodiments, the hydrophilic block of the block copolymers is polycationic at about neutral pH. In certain embodiments, the hydrophilic block comprises cationic and non-cationic monomeric units. In some embodiments, the hydrophilic block comprises at least one cationic chargeable monomeric unit and at least one non-chargeable monomeric unit.

In some embodiments, a hydrophilically-shielded micelle having membrane-destabilizing copolymers provided herein comprises a plurality of block copolymers with a hydrophilic block that is a homopolymeric block. In further or alternative embodiments, a hydrophilically-shielded micelle having membrane-destabilizing copolymers provided herein comprises a plurality of block copolymer with a hydrophilic block that is a heteropolymeric block. In some embodiments, the hydrophilic block of a block copolymer of a micelle provided herein comprises a N,N-di(C₁-C₆)alkyl-amino(C₁-C₆)alkyl-ethacrylate monomeric unit, a N,N-di(C₁-C₆)alkyl-amino(C₁-C₆)alkyl-methacrylate monomeric unit, a N,N-di(C₁-C₆)alkyl-amino(C₁-C₆)alkyl-acrylate monomeric unit, or a combination thereof.

In some embodiments, a hydrophilically-shielded micelle having membrane-destabilizing copolymers provided herein comprises a core with at least one first chargeable species and at least one second chargeable species, wherein the first chargeable species is chargeable or charged to an anionic species, wherein the second chargeable species is chargeable or charged to a cationic species, and wherein the ratio of first chargeable species to second chargeable species present in the core is about 1:4 to about 4:1. In some embodiments, the ratio of positively charged groups to negatively charged groups in the core is about 1:4 to about 4:1 at about neutral pH. In certain embodiments, the ratio of positively charged groups to negatively charged groups in the core is about 1:2 to about 2:1 at about neutral pH. In some embodiments, the ratio of positively charged groups to negatively charged groups in the core is about 1:1.1 to about 1.1:1 at about neutral pH.

In some embodiments, the hydrophobic block of the block copolymer comprises more than 5, more than 20, more than 50, or more than 100 chargeable species that are charged or chargeable to anionic species. In some embodiments, the hydrophobic block of the block copolymer comprises more than 5, more than 20, more than 50, or more than 100 first chargeable species. In specific embodiments, each first chargeable species is chargeable or charged to an anionic species. In some embodiments, the hydrophobic block of the block copolymer comprises more than 5, more than 20, more than 50, or more than 100 second chargeable species. In specific embodiments, each second chargeable species is charged or chargeable to a cationic species. In certain embodiments, the hydrophobic block of the block copolymer comprises more than 5, more than 20, more than 50, or more than 100 hydrophobic species. In some embodiments, the hydrophobic block copolymer comprises more than 5, more than 20, more than 50, or more than 100 chargeable species that are charged or chargeable to anionic species. In certain embodiments, the hydrophobic block of the block copolymer provided herein comprises more than 5, more than 20, more than 50, or more than 100 first chargeable species. In specific embodiments, each first chargeable species is chargeable or charged to an anionic species. In some embodiments, the hydrophobic block of the block copolymer comprises more than 5, more than 20, more than 50, or more than 100 second chargeable species. In specific embodiments, each second chargeable species is charged or chargeable to a cationic species. In certain embodiments, the hydrophobic block of the block copolymer provided herein comprises more than 5, more than 20, more than 50, or more than 100 hydrophobic species.

In some embodiments, a hydrophobic block comprises a first chargeable species (e.g., anionic chargeable) present on a first monomeric unit and the second chargeable species (e.g., cationic chargeable) on a second monomeric unit. In alternative embodiments, a first and second chargeable species are on the same monomeric unit (e.g., a zwitteroinically chargeable monomeric unit). In some embodiments, the ratio of the number of first monomeric units to the number of second monomeric units present in the core is about 1:4 to about 4:1.

In certain embodiments, a hydrophilically-shielded micelle having membrane-destabilizing copolymers provided herein comprises at least one block copolymer with a hydrophobic block that comprises at least one first chargeable monomeric unit and at least one second chargeable monomeric unit. In some embodiments, the first chargeable monomeric unit is Brønsted acid. In certain embodiments, at least 80% of the first chargeable monomeric unit is charged, by loss of a H⁺, to an anionic species at a pH of about 7.4. In further or alternative embodiments, less than 50% of the first chargeable monomeric unit is charged to an anionic species at a pH of about 6. In some embodiments, the first chargeable monomeric unit is a (C₂-C₈)alkylacrylic acid. In certain embodiments, the second chargeable monomeric unit is a Brønsted base. In some embodiments, at least 40% of the second chargeable monomeric unit is charged, by gain of a H⁺, to a cationic species at a pH of about 7.4. In certain embodiments, the second chargeable monomeric unit is N,N-di(C₁-C₆)alkyl-amino(C₁-C₆)alkyl-ethacrylate, N,N-di(C₁-C₆)alkyl-amino(C₁-C₆)alkyl-methacrylate, or N,N-di(C₁-C₆)alkyl-amino(C₁-C₆)alkyl-acrylate. In some embodiments, the hydrophobic block further comprises at least one non-chargeable monomeric unit. In certain embodiments, the non-chargeable monomeric unit is a (C₂-C₈)alkyl-ethacrylate, a (C₂-C₈)alkyl-methacrylate, or a (C₂-C₈)alkyl-acrylate.

In some embodiments a hydrophilically-shielded micelle having membrane-destabilizing copolymers provided herein is a particle with an average hydrodynamic diameter of about 10 nm to about 200 nm. In specific embodiments, the micelle has an average hydrodynamic diameter of about 20 nm to about 100 nm. In more specific embodiments, the micelle has an average hydrodynamic diameter of about 30 nm to about 80 nm.

In some embodiments, provided herein is a hydrophilically-shielded micelle having membrane-destabilizing copolymers that is self-assembled. In certain embodiments, the micelle self-assembles in an aqueous medium at a pH within about 6.5 to about 7.5. In some embodiments, the self-assembly occurs in less than 2 hours, in less than 1 hour, in less than 30 minutes, in less than 15 minutes. In some embodiments, the micelle is membrane destabilizing in an aqueous medium at a pH within about 5.0 to about 7.4. In some embodiments, micelle formation occurs in the absence of the nucleic acid. In some embodiments, micelle formation occurs in the presence of nucleic acid. In some embodiments, micelle formation occurs in the presence or absence of nucleic acid.

In certain embodiments, provided herein is a hydrophilically-shielded micelle having membrane-destabilizing copolymers that comprises a greater net cationic charge at pH of about 5 than at a pH of about 7. In some embodiments, the absolute value of the charge of the micelle is greater at pH of about 5 than at a pH of about 7.

In some embodiments, provided herein is a hydrophilically-shielded micelle having membrane-destabilizing copolymers comprising a plurality of block copolymers having a hydrophobic block and a hydrophilic block, wherein the ratio of the number average molecular weight of the hydrophobic block to the number average molecular weight of the hydrophilic block is about 5:1 to about 1:1, or from 1:1 to about 5:1. In more specific embodiments, the ratio of the number average molecular weight of the hydrophobic block to the number average molecular weight of the hydrophilic block is about 2:1.

In certain embodiments, the micelle provided herein comprises a plurality of block copolymers with a hydrophobic block having a number average molecular weight (Mn) of about 2,000 dalton to about 200,000 dalton, about 2,000 dalton to about 100,000 dalton, or about 10,000 dalton to about 200,000 dalton. In some embodiments, the micelle provided herein comprises a plurality of block copolymers with a hydrophilic block having a number average molecular weight (Mn) of about 5,000 dalton to about 50,000 dalton. In some embodiments, the micelle provided herein comprises a plurality of block copolymers with a hydrophobic block having a number average molecular weight (Mn) of greater than 200,000 dalton. In some embodiments, the micelle provided herein comprises a plurality of block copolymers with a hydrophobic block having a number average molecular weight (Mn) of greater than 100,000 dalton. In some embodiments, the micelle provided herein comprises a plurality of block copolymers with a hydrophilic block having a number average molecular weight (Mn) of greater than 50,000 dalton.

In some embodiments, the block copolymers provided herein have a polydispersity index of less than 2, less than 1.8, less than 1.6, less than 1.5, less than 1.4, or less than 1.3.

In some embodiments, provided herein is a hydrophilically-shielded micelle having membrane-destabilizing copolymers that is stable at a pH of about 7.4. In certain embodiments, the micelle is substantially less stable at a pH of about 5.8 than at a pH of about 7.4.

In certain embodiments, provided herein is a hydrophilically-shielded micelle having membrane-destabilizing copolymers that is stable at a concentration of about 10 μg/mL, or greater (e.g., at about neutral pH). In some embodiments, provided herein is a hydrophilically-shielded micelle having membrane-destabilizing copolymers that is stable at a concentration of about 100 μg/mL, or greater (e.g., at about neutral pH).

In certain embodiments described herein are any of the polymers that make up the micelles described herein. That is, the polymeric subunits (e.g., the block copolymers) or the individual polymers (whether or not in the form of a micelle) are also embodiments described herein. To be explicit, each and every block copolymer that is presented herein is within the scope of the inventions described herein, both as an individual polymer, or as a polymeric unit/strand/component of the micelle described herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: An illustrative example of the composition and properties of PEGMA-DMAEMA copolymers

FIG. 2: An illustrative example of the galactose end functionalized poly[DMAEMA]-macro CTA

FIG. 3: An illustrative example of the synthesis of [PEGMA-MAA(NHS)]-[B-P-D]

FIG. 4: An illustrative example of the RAFT Co-polymerization of PEGMA and MAA-NHS

FIG. 5: An illustrative example of the galactose functionalized DMAEMA-MAA(NHS) or PEGMA-MAA(NHS) di-block co-polymers

FIG. 6: An illustrative example of the structures of conjugatable siRNAs, peptides, and pyridyl disulfide amine

### DETAILED DESCRIPTION OF THE INVENTION

Provided herein are polymeric micelles (i.e., micelles comprising polymers) that are composed of a plurality of hydrophilically-shielded membrane-destabilizing block copolymers. That is, the block copolymers that comprise the micelle comprise both a hydrophilic shielding portion and a pH-dependent membrane-destabilizing portion. The block copolymers optionally include further portions, but at the least the block copolymers have both of the aforementioned portions. The hydrophilic shielding portion of the block copolymer is comprised of constitutional units with a hydrophilic pendant group, including with a polyoxylated alkyl pendant group, and the pH dependent membrane destabilizing portion is a hydrophobic copolymer block that comprises a first chargeable species that is anionic at about neutral pH. When the pH is at about the pKₐ of the chargeable species, there will exist an equilibrium distribution of chargeable species in both forms. In the case of an anionic species, about 50% of the population will be anionic and about 50% will be non-charged when the pH is at the pKₐ of the anionic species. The further the pH is from the pKₐ of the chargeable species, there will be a corresponding shift in this equilibrium such that at higher pH values, the anionic form will predominate and at lower pH values, the uncharged form will predominate. The embodiments described herein include the form of the copolymers at any pH value.

In some embodiments the micelle further includes a therapeutic agent or some other micellar payload and the hydrophilic shielding portion enhances the stability of this payload (e.g., polynucleotide or peptide, etc.), including shielding the payload against enzymatic-based digestion. In some instances, a shielding agent reduces toxicity of micelles or the hydrophilically-shielded membrane-destabilizing block copolymers described herein. In some instances, a shielding agent provides the micelle with desirable surface properties. In some instances a shielding agent is in the hydrophilic block of a block copolymer. In certain embodiments, the hydrophilic shielding portion also includes a cationic polynucleotide carrier block/segment, and the hydrophilic shielding serves to shield, at least in part, the charge (e.g., cationic charges) on the polynucleotide carrier block/segment.

The hydrophilic shielding results, at least in part, from the presence of a hydrophilic pendant group on at least some of the constitutional units that make up the hydrophilic shielding portion of the block copolymers. In some embodiments, the aforementioned hydrophilic pendant groups are also found on the monomers that are used to produce the hydrophilic block copolymer. That is, in particular embodiments, the hydrophilic groups are not added to the polymer post-polymerization, but rather incorporated into the polymer via the hydrophilic pendant groups of the monomers. However, the hydrophilic pendant groups of the monomer and on the polymer need to be strictly identical, for example, the monomers may have a protected form of the hydrophilic pendant group, and following polymerization, the protecting group is removed.

One example of a pendant hydrophilic group is Y₆-Q₆: Y₆ is selected from the group consisting of a covalent bond, (1C-10C)alkyl-, —C(O)O(2C-10C) alkyl-, —OC(O)(1C-10C) alkyl-, —O(2C-10C)alkyl- and —S(2C-10C)alkyl- —C(O)NR₆(2C-10C) alkyl-; Q₆ is a residue selected from the group consisting of residues which are hydrophilic at physiologic pH and are substantially non-charged at physiologic pH (e.g., hydroxy, polyoxylated alkyl, polyethylene glycol, polypropylene glycol, thiol, or the like).

In one particular series of embodiments, the pendant group on the polymer and on the monomer share the following structural feature:

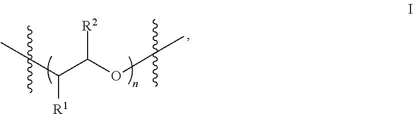

where R¹ and R² are each independently selected from the group consisting of hydrogen, halogen, C₁-C₃ haloalkyl, and optionally substituted C₁-C₃ alkyl, and n is an integer ranging from 2 to 20.

In some instances, provided herein are micelles suitable for the delivery of therapeutic agents (including, e.g., oligonucleotides or peptides) to a living cell. In some embodiments, the micelles comprise a plurality of block copolymers and, optionally, at least one therapeutic agent. In certain embodiments, the micelles provided herein are biocompatible, stable (including chemically and/or physically stable), and/or reproducibly synthesized. Additionally, in some embodiments, the micelles provided herein are non-toxic (e.g., exhibit low toxicity), protect the therapeutic agent (e.g., oligonucleotide or peptide) payload from degradation, enter living cells via a naturally occurring process (e.g., by endocytosis), and/or deliver the therapeutic agent (e.g., oligonucleotide or peptide) payload into the cytoplasm of a living cell after being contacted with the cell. In certain instances, the polynucleotide (e.g., oligonucleotide) is an siRNA and/or another ‘nucleotide-based’ agent that alters the expression of at least one gene in the cell. Accordingly, in certain embodiments, the micelles provided herein are useful for delivering siRNA or peptide into a cell. In certain instances, the cell is in vitro, and in other instances, the cell is in vivo. In some embodiments, a therapeutically effective amount of the micelles comprising an siRNA or peptide is administered to an individual in need thereof (e.g., in need of having a gene knocked down, wherein the gene is capable of being knocked down by the siRNA administered). In specific instances, the micelles are useful for or are specifically designed for delivery of siRNA or peptide to specifically targeted cells of the individual.

### DEFINITIONS

It is understood that, with regard to this application, use of the singular includes the plural and vice versa unless expressly stated to be otherwise. That is, “a” and “the” refer to one or more of whatever the word modifies. For example, “the polymer” or “a nucleotide” may refer to one polymer or nucleotide or to a plurality of polymers or nucleotides. By the same token, “polymers” and “nucleotides” would refer to one polymer or one nucleotide as well as to a plurality of polymers or nucleotides unless, again, it is expressly stated or obvious from the context that such is not intended.

As used herein, two groups (e.g., an siRNA and a hydrophilic block) are “associated” or “attached” if they are held together by any interaction including, by way of non-limiting example, one or more covalent bonds, one or more non-covalent interactions (e.g., ionic bonds, static forces, van der Waals interactions, combinations thereof, or the like), or a combination thereof.

Anionic monomer: “Anionic monomer” or “anionic monomeric unit”, as used herein, is a monomer or monomeric unit bearing a group that is present in an anionic charged state or in a non-charged state, but in the non-charged state is capable of becoming anionic charged, e.g., upon removal of an electrophile (e.g., a proton (H⁺), for example in a pH dependent manner). In certain instances, the group is substantially negatively charged at an approximately physiological pH but undergoes protonation and becomes substantially neutral at a weakly acidic pH. The non-limiting examples of such groups include carboxyl groups, barbituric acid and derivatives thereof, xanthine and derivatives thereof, boronic acids, phosphinic acids, phosphonic acids, sulfinic acids, phosphates, and sulfonamides.

Anionic species: “Anionic species”, as used herein, is a group, residue or molecule that is present in an anionic charged or non-charged state, but in the non-charged state is capable of becoming anionic charged, e.g., upon removal of an electrophile (e.g., a proton (H⁺), for example in a pH dependent manner). In certain instances, the group, residue or molecule is substantially negatively charged at an approximately physiological pH but undergoes protonation and becomes substantially neutral at a weakly acidic pH.

As used herein, a “charge neutralized” means a particle having a Zeta potential that is between ±10 to ±30 mV, and/or the presence of a first number (z) of chargeable species that are chargeable to a negative charge (e.g., acidic species that become anionic upon de-protonation) and a second number (0.5·z) of chargeable species that are chargeable to a positive charge (e.g., basic species that become cationic upon protonation).

As used herein, normal physiological pH refers to the pH of the predominant fluids of the mammalian body such as blood, serum, the cytosol of normal cells, etc. In certain instances, normal physiologic pH is about neutral pH, including, e.g., a pH of about 7.2 to about 7.4. In some instances, about neutral pH is a pH of 6.6 to 7.6. As used herein, the terms neutral pH, physiologic and physiological pH are synonymous and interchangeable.

As used herein, a micelle is “disrupted” if it does not function in an identical, substantially similar or similar manner and/or possess identical, substantially similar or similar physical and/or chemical characteristics as would a stable micelle in an aqueous solution representing physiological conditions, for example phosphate-buffered saline at pH 7.4. Micelle stability can be quantitatively defined by the critical micelle concentration (CMC), defined as the micelle concentration where instability occurs, as indicated by uptake of a hydrophobic probe molecule (e.g., the pyrene fluorescence assay) or changes in the size of the micelle (e.g., as determined by dynamic light scattering measurements). In “disruption” of a micelle can be determined in any suitable manner. In one instance, a micelle is “disrupted” if it does not have a hydrodynamic particle size that is less than 5 times, 4 times, 3 times, 2 times, 1.8 times, 1.6 times, 1.5 times, 1.4 times, 1.3 times, 1.2 times, or 1.1 times the hydrodynamic particle size of a micelle comprising the same block copolymers and as formed in an aqueous solution at a pH of 7.4, or formed in human serum. In one instance, a micelle is “disrupted” if it does not have a concentration of assembly that is less than 5 times, 4 times, 3 times, 2 times, 1.8 times, 1.6 times, 1.5 times, 1.4 times, 1.3 times, 1.2 times, or 1.1 times the concentration of assembly of a micelle comprising the same block copolymers and as formed in an aqueous solution at a pH of 7.4, or formed in human serum.

As used herein, a “chargeable species”, “chargeable group”, or “chargeable monomeric unit” is a species, group or monomeric unit in either a charged or non-charged state. In certain instances, a “chargeable monomeric unit” is one that can be converted to a charged state (either an anionic or cationic charged state) by the addition or removal of an electrophile (e.g., a proton (H⁺), for example in a pH dependent manner). The use of any of the terms “chargeable species”, “chargeable group”, or “chargeable monomeric unit” includes the disclosure of any other of a “chargeable species”, “chargeable group”, or “chargeable monomeric unit” unless otherwise stated.

Hydrophobic species: “hydrophobic species” (used interchangeably herein with “hydrophobicity-enhancing moiety”), as used herein, is a moiety such as a substituent, residue or a group which, when covalently attached to a molecule, such as a monomer or a polymer, increases the molecule's hydrophobicity or serves as a hydrophobicity enhancing moiety. The term “hydrophobicity” is a term of art describing a physical property of a compound measured by the free energy of transfer of the compound between a non-polar solvent and water (Hydrophobicity regained. Karplus P. A., Protein Sci., 1997, 6: 1302-1307.) A compound's hydrophobicity can be measured by its log P value, the logarithm of a partition coefficient (P), which is defined as the ratio of concentrations of a compound in the two phases of a mixture of two immiscible solvents, e.g. octanol and water. Experimental methods of determination of hydrophobicity as well as methods of computer-assisted calculation of log P values are known to those skilled in the art. Hydrophobic species of the present invention include but are not limited to aliphatic, heteroaliphatic, aryl, and heteroaryl groups.

Without being bound by theory not expressly recited in the claims, a membrane destabilizing polymer can directly or indirectly elicit a change (e.g., a permeability change) in a cellular membrane structure (e.g., an endosomal membrane) so as to permit an agent (e.g., polynucleotide), in association with or independent of a micelle (or a constituent polymer thereof), to pass through such membrane structure—for example to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or disruption of a cellular membrane (e.g., as observed for a substantial fraction of a population of cellular membranes).

Generally, membrane destabilizing or membrane disruptive properties of polymers or micelles can be assessed by various means. In one non-limiting approach, a change in a cellular membrane structure can be observed by assessment in assays that measure (directly or indirectly) release of an agent (e.g., polynucleotide) from cellular membranes (e.g., endosomal membranes)—for example, by determining the presence or absence of such agent, or an activity of such agent, in an environment external to such membrane. Another non-limiting approach involves measuring red blood cell lysis (hemolysis)—e.g., as a surrogate assay for a cellular membrane of interest. Such assays are optionally conducted at a single pH value or over a range of pH values.

As used herein, a “micelle” includes a particle comprising a core and a hydrophilic shell, wherein the core is held together at least partially, predominantly or substantially through hydrophobic interactions. In certain instances, as used herein, a “micelle” is a multi-component, nanoparticle comprising at least two domains, the inner domain or core, and the outer domain or shell. The core is at least partially, predominantly or substantially held together by hydrophobic interactions, and is present in the center of the micelle. As used herein, the “shell of a micelle” is defined as non-core portion of the micelle.

A “pH dependent membrane-destabilizing portion” is a group that is at least partially, predominantly, or substantially hydrophobic and is membrane destabilizing in a pH dependent manner. In certain instances, a pH dependent membrane destabilizing portion is a hydrophobic polymeric segment of a block copolymer and/or comprises a plurality of hydrophobic species; and comprises a plurality of anionic species. In some embodiments, the anionic species is anionic at about neutral pH. In further or alternative embodiments, the anionic species is non-charged at a lower, e.g., endosomal pH. In some embodiments, the membrane destabilizing portion comprises a plurality of cationic species. The pH dependent membrane-destabilizing portion has neither a non-peptidic and non-lipidic polymer backbone.

Nanoparticle: As used herein, the term “nanoparticle” refers to any particle having a diameter of less than 1000 nanometers (nm). In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g. having diameters of about 10 nm to about 200 nm, about 20 nm to about 100 nm, or 50 nm or less, e.g., 5 nm-30 nm, are used in some embodiments.

Nucleotide: As used herein, the term “nucleotide,” in its broadest sense, refers to any compound and/or substance that is or can be incorporated into a polynucleotide (e.g., oligonucleotide) chain. In some embodiments, a nucleotide is a compound and/or substance that is or can be incorporated into a polynucleotide (e.g., oligonucleotide) chain via a phosphodiester linkage. In some embodiments, “nucleotide” refers to individual nucleic acid residues (e.g. nucleotides and/or nucleosides). In certain embodiments, “at least one nucleotide” refers to one or more nucleotides present; in various embodiments, the one or more nucleotides are discrete nucleotides, are non-covalently attached to one another, or are covalently attached to one another. As such, in certain instances, “at least one nucleotide” refers to one or more polynucleotide (e.g., oligonucleotide). In some embodiments, a polynucleotide is a polymer comprising two or more nucleotide monomeric units.

Oligonucleotide gene expression modulator: as used herein, an “oligonucleotide gene expression modulator” is an oligonucleotide agent capable of inducing a selective modulation of gene expression in a living cell by mechanisms including but not limited to an antisense mechanism or by way of an RNA interference (RNAi)-mediated pathway which may include (i) transcription inactivation; (ii) mRNA degradation or sequestration; (iii) transcriptional inhibition or attenuation or (iv) inhibition or attenuation of translation. Oligonucleotide gene expression modulators include, regulatory RNA (including virtually any regulatory RNA) such as, but not limited to, antisense oligonucleotides, miRNA, siRNA, RNAi, shRNA, aptamers and any analogs or precursors thereof.

Oligonucleotide knockdown agent: as used herein, an “oligonucleotide knockdown agent” is an oligonucleotide species which can inhibit gene expression by targeting and binding an intracellular nucleic acid in a sequence-specific manner. Non-limiting examples of oligonucleotide knockdown agents include siRNA, miRNA, shRNA, dicer substrates, antisense oligonucleotides, decoy DNA or RNA, antigene oligonucleotides and any analogs and precursors thereof.

As used herein, the term “oligonucleotide” refers to a polymer comprising 7-200 nucleotide monomeric units. In some embodiments, “oligonucleotide” encompasses single and or/double stranded RNA as well as single and/or double-stranded DNA. Furthermore, the terms “nucleotide”, “nucleic acid,” “DNA,” “RNA,” and/or similar terms include nucleic acid analogs, i.e. analogs having a modified backbone, including but not limited to peptide nucleic acids (PNA), locked nucleic acids (LNA), phosphono-PNA, morpholino nucleic acids, or nucleic acids with modified phosphate groups (e.g., phosphorothioates, phosphonates, 5′-N-phosphoramidite linkages). Nucleotides can be purified from natural sources, produced using recombinant expression systems and optionally purified, chemically synthesized, etc. As used herein, a “nucleoside” is the term describing a compound comprising a monosaccharide and a base. The monosaccharide includes but is not limited to pentose and hexose monosaccharides. The monosaccharide also includes monosaccharide mimetics and monosaccharides modified by substituting hydroxyl groups with halogens, methoxy, hydrogen or amino groups, or by esterification of additional hydroxyl groups. In some embodiments, a nucleotide is or comprises a natural nucleoside phosphate (e.g. adenosine, thymidine, guanosine, cytidine, uridine, deoxyadenosine, deoxythymidine, deoxyguanosine, and deoxycytidine phosphate). In some embodiments, the base includes any bases occurring naturally in various nucleic acids as well as other modifications which mimic or resemble such naturally occurring bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, 2-aminoadenine, pyrrolopyrimidine, and 2,6-diaminopurine. Nucleoside bases also include universal nucleobases such as difluorotolyl, nitroindolyl, nitropyrrolyl, or nitroimidazolyl. Nucleotides also include nucleotides which harbor a label or contain abasic, i.e. lacking a base, monomers. A nucleic acid sequence is presented in the 5′ to 3′ direction unless otherwise indicated. A nucleotide can bind to another nucleotide in a sequence-specific manner through hydrogen bonding via Watson-Crick base pairs. Such base pairs are said to be complementary to one another. An oligonucleotide can be single stranded, double-stranded or triple-stranded.

RNA interference (RNAi): As used herein, the term “RNA interference” or “RNAi” refers to sequence-specific inhibition of gene expression and/or reduction in target mRNA and protein levels mediated by an at least partially double-stranded RNA, which also comprises a portion that is substantially complementary to a target RNA.

RNAi agent: As used herein, the term “RNAi agent” refers to an oligonucleotide which can mediate inhibition of gene expression through an RNAi mechanism and includes but is not limited to siRNA, microRNA (miRNA), short hairpin RNA (shRNA), asymmetrical interfering RNA (aiRNA), dicer substrate and the precursors thereof.

Short interfering RNA (siRNA): As used herein, the term “short interfering RNA” or “siRNA” refers to an RNAi agent comprising a nucleotide duplex that is approximately 15-50 base pairs in length and optionally further comprises zero to two single-stranded overhangs. One strand of the siRNA includes a portion that hybridizes with a target RNA in a complementary manner. In some embodiments, one or more mismatches between the siRNA and the targeted portion of the target RNA may exist. In some embodiments, siRNAs mediate inhibition of gene expression by causing degradation of target transcripts.

Short hairpin RNA (shRNA): Short hairpin RNA (shRNA) refers to an oligonucleotide having at least two complementary portions hybridized or capable of hybridizing with each other to form a double-stranded (duplex) structure and at least one single-stranded portion.

Dicer Substrate: a “dicer substrate” is a greater than approximately 25 base pair duplex RNA that is a substrate for the RNase III family member Dicer in cells. Dicer substrates are cleaved to produce approximately 21 base pair duplex small interfering RNAs (siRNAs) that evoke an RNA interference effect resulting in gene silencing by mRNA knockdown.

As used herein, a “substantially non-charged” includes a Zeta potential that is between ±10 to ±30 mV, and/or the presence of a first number (z) of chargeable species that are chargeable to a negative charge (e.g., acidic species that become anionic upon de-protonation) and a second number (0.5·z) of chargeable species that are chargeable to a positive charge (e.g., basic species that become cationic upon protonation).

Therapeutic agent: As used herein, the phrase “therapeutic agent” refers to any agent that, when administered to a subject, organ, tissue, or cell has a therapeutic effect and/or elicits a desired biological and/or pharmacological effect.

### Micelle Structure

Provided herein are polymeric micelles (i.e., micelles comprising polymers) that are composed of a plurality of hydrophilically-shielded membrane-destabilizing block copolymers. That is, the block copolymers that comprise the micelle comprise both a hydrophilic shielding portion and a pH-dependent membrane-destabilizing portion. The block copolymers optionally include further portions, but at the least the block copolymers have both of the aforementioned portions. The hydrophilic shielding portion of the block copolymer is comprised of constitutional units with a hydrophilic pendant group, including with a polyoxylated alkyl pendant group, and the pH dependent membrane destabilizing portion is a hydrophobic copolymer block that comprises a first chargeable species that is anionic at about neutral pH.

The hydrophilic shielding results, at least in part, from the presence of a hydrophilic pendant group on at least some of the constitutional units that make up the hydrophilic shielding portion of the block copolymers. In some embodiments, the aforementioned hydrophilic pendant groups are also found on the monomers that are used to produce the hydrophilic block copolymer. That is, in particular embodiments, the hydrophilic groups are not added to the polymer post-polymerization, but rather incorporated into the polymer via the hydrophilic pendant groups of the monomers. However, the hydrophilic pendant groups of the monomer and on the polymer need to be strictly identical, for example, the monomers may have a protected form of the hydrophilic pendant group, and following polymerization, the protecting group is removed. In one particular series of embodiments, the pendant group on the polymer and on the monomer share the following structural feature:

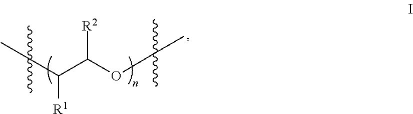

where R¹ and R² are each independently selected from the group consisting of hydrogen, halogen, C₁-C₃ haloalkyl, and optionally substituted C₁-C₃ alkyl, and n is an integer ranging from 2 to 20.

Provided in certain embodiments herein are hydrophilically-shielded micelles having membrane-destabilizing copolymers and processes for making the same. In some embodiments, a hydrophilically-shielded micelle having membrane-destabilizing copolymers provided herein comprises a plurality of block copolymers, the block copolymers comprising a hydrophilic block and a hydrophobic block. In some embodiments, the micelle comprising a core and a shell, wherein the core comprises a hydrophobic block of the multiblock polymer, and wherein the shell comprises a hydrophilic block of the multiblock polymer. In some embodiments, the micelles described herein are self-assembled. In specific embodiments, the micelles are spontaneously self-assembled.

Provided in some embodiments herein is a hydrophilically-shielded micelle having membrane-destabilizing copolymers comprising a plurality of block copolymers. In certain embodiments, the micelle comprises a core and a shell.

In certain embodiments, the micelle comprises a plurality of membrane-destabilizing block copolymers. As used herein, membrane-destabilizing block copolymers include membrane-disruptive block copolymers (e.g., polymers that lyse an endosomal membrane) and block copolymers that locally destabilize a membrane (e.g., via a temporary rift in an endosomal membrane). In some embodiments, a membrane-destabilizing block copolymer comprises (i) a plurality of hydrophobic monomeric residues, (ii) a plurality of anionic monomeric residues having a chargeable species, the chargeable species being anionic at serum physiological pH, and being substantially neutral or non-charged at an endosomal pH and (iii) optionally a plurality of cationic monomeric residues. In some embodiments, modification of the ratio of anionic to cationic species in a block copolymer allows for modification of membrane destabilizing activity of a micelle described herein. In some of such embodiments, the ratio of anionic:cationic species in a block copolymer ranges from about 4:1 to about 1:4 at serum physiological pH. In some of such embodiments, modification of the ratio of anionic to cationic species in a hydrophobic block of a block copolymer allows for modification of membrane destabilizing activity of a micelle described herein. In some of such embodiments, the ratio of anionic:cationic species in a hydrophobic block of a block copolymer described herein ranges from about 1:2 to about 3:1, or from about 1:1 to about 2:1 at serum physiological pH.

In certain embodiments, the copolymers present in a hydrophilically-shielded micelle having membrane-destabilizing copolymers provided herein comprise a core section (e.g., hydrophobic block) that comprises a plurality of hydrophobic groups. In more specific embodiments, the core section (e.g., hydrophobic block) comprises a plurality of hydrophobic groups and a plurality of first chargeable species or groups. In still more specific embodiments, such first chargeable species or groups are negatively charged and/or are chargeable to a negatively charged species or group (e.g., at about a neutral pH, or a pH of about 7.4). In some specific embodiments, the core section (e.g., hydrophobic block) comprises a plurality of hydrophobic groups, a plurality of first chargeable species or groups, and a plurality of second chargeable species or groups. In more specific embodiments, the first chargeable species or groups are negatively charged and/or are chargeable to a negatively charged species or group, and the second chargeable species or groups are positively charged and/or are chargeable to a positively charged species or group (e.g., at about a neutral pH, or a pH of about 7.4).

In certain embodiments, the core of the micelle comprises a plurality of hydrophobic groups. In some embodiments, the hydrophobic groups are hydrophobic about at a neutral pH. In more specific embodiments, the hydrophobic group is hydrophobic at a slightly acidic pH (e.g., at a pH of about 6 and/or a pH of about 5). In certain embodiments, two or more different hydrophobic groups are present. In some embodiments, a hydrophobic group has a π value of about one, or more. A compound's π value is a measure of its relative hydrophilic-lipophilic value (see, e.g., Cates, L. A., “Calculation of Drug Solubilities by Pharmacy Students” Am. J. Pharm. Educ. 45:11-13 (1981)).

In some embodiments, the core of the micelle comprises at least one charge at about a neutral pH (e.g., about 7.4). In specific embodiments, at least one charge is a negative charge. In a more specific embodiment, at least one charge is at least one negative charge and at least two positive charges.

In some embodiments, the hydrophobic blocks of the block copolymers are membrane destabilizing. In specific embodiments, the hydrophobic block of the block copolymers described herein is a pH dependent membrane destabilizing hydrophobe. In specific embodiments, the hydrophilic block is hydrophilic at about a neutral pH.

In certain embodiments, the shell of the micelle and/or the hydrophilic blocks described herein also comprise a chargeable species or groups. In some embodiments, one or more copolymers present in a micelle provided herein has a shell section that comprises a plurality of cationically chargeable species or groups. Depending on the concentration of electrolytes in a medium surrounding the micelle (e.g., on the pH), these cationically chargeable species are either in a cationically charged, or in a non-charged state.

In certain embodiments, a hydrophilically-shielded micelle having membrane-destabilizing copolymers provided herein has a net cationic charge at a pH of about 5. In some embodiments, a hydrophilically-shielded micelle having membrane-destabilizing copolymers described herein has a net neutral charge at about a neutral pH. In certain embodiments, a hydrophilically-shielded micelle having membrane-destabilizing copolymers described herein has a net cationic charge at about neutral pH (e.g., at a pH of about 7.4). In some embodiments, a hydrophilically-shielded micelle having membrane-destabilizing copolymers described herein has a greater net cationic charge at pH of about 5 than at a pH of about 7. In further or alternative embodiments, a hydrophilically-shielded micelle having membrane-destabilizing copolymers provided herein has a nominal (or absolute value of) charge that is greater at pH of about 5 than at a pH of about 7. In some embodiments, the Zeta potential of the micelle is charge neutralized.

In certain embodiments, provided herein is a hydrophilically-shielded micelle having membrane-destabilizing copolymers wherein the form of the micelle is a micelle, a pseudo-micelle, or a micelle-like structure over the pH range of about 6 and up, about 6.5 and up, about 7 and up, about 6 to about 14, or more; about 6 to about 10, or more; about 6 to about 9.5, or more; about 6 to about 9, or more; about 6 to about 8.5, or more; about 6 to about 8, or more; about 6.5 to about 14, or more; about 6.5 to about 10, or more; about 6.5 to about 9.5, or more; about 6.5 to about 9, or more; about 6.5 to about 8.5, or more; about 7 to about 14, or more; about 7 to about 10, or more; about 7 to about 9.5, or more; about 7 to about 9, or more; about 7 to about 8.5, or more; about 6.2 to about 7.5, or more; 6.2 to 7.5; or about 7.2 to about 7.4. In certain embodiments, at a pH of about 7, or below; about 6.8, or below; about 6.5, or below; about 6.2, or below; about 6, or below; about 5.8, or below; or about 5.7, or below, the micelle, micelle, pseudo-micelle, or micelle-like structure provided herein become substantially, or at least partially disrupted or disassociated. In specific embodiments, the form of the micelle over the pH range of about 6.2 to 7.5 is a micelle. It is to be understood that as used herein, the micelles have a form over at least the pH described and may also have the described form at a pH outside the pH range described.

In some instances, the micelles provided herein are formed from a plurality of block copolymers which self-associate. In certain instances, the self-association occurs through the interactions of the hydrophobic blocks of the block copolymers and the resulting micelles are stabilized through hydrophobic interactions of the hydrophobic blocks present in the core of the micelles.

In some embodiments, the micelles provided herein retain activity (e.g., the activity of the micelle to deliver a therapeutic agent, e.g., a polynucleotide) in 50% human serum for at least 2 hours, at least 4 hours, at least 6 hours, at least 8 hours, at least 12 hours, or at least 24 hours. In further or alternative embodiments, the micelles provided herein retain activity (e.g., the activity of the micelle to deliver a polynucleotide) in at least 50% human plasma for at least 2 hours, at least 4 hours, at least 6 hours, at least 8 hours, at least 12 hours, or at least 24 hours. In further or alternative embodiments, the micelles provided herein retain activity (e.g., the activity of the micelle to deliver a polynucleotide) in 50% mouse serum for at least 2 hours, at least 4 hours, at least 6 hours, at least 8 hours, at least 12 hours, or at least 24 hours. In still further or alternative embodiments, the micelles provided herein retain activity (e.g., the activity of the micelle to deliver a therapeutic agent, e.g., a polynucleotide) in at least 50% mouse plasma for at least 2 hours, at least 4 hours, at least 6 hours, at least 8 hours, at least 12 hours, or at least 24 hours. In specific embodiments, the micelles provided herein retain activity (e.g., the activity of the micelle to deliver a therapeutic agent, e.g., a polynucleotide) in 50% human serum for at least 2 hours, in at least 50% human plasma for at least 2 hours, in 50% mouse serum for at least 2 hours, in at least 50% mouse plasma for at least 2 hours, or a combination thereof.

In some embodiments, a hydrophilically-shielded micelle having membrane-destabilizing copolymers provided herein is characterized by one or more of the following: (1) the micelle is formed by spontaneous self association of block copolymers to form organized assemblies (e.g., micelles) upon dilution from a water-miscible solvent (such as but not limited to ethanol) to aqueous solvents (for example phosphate-buffered saline, pH 7.4); (2) the micelle is stable to dilution (e.g., down to a polymer concentration of 100 ug/ml, 50 ug/ml, 10 ug/ml, 5 ug/ml or 1 ug/ml, which constitutes the critical stability concentration or the critical micelle concentration (CMC)); (3) the micelle is stable to high ionic strength of the surrounding media (e.g. 0.5M NaCl); and/or (4) the micelle has an increasing instability as the concentration of organic solvent increases, such organic solvents including, but not limited to dimethylformamide (DMF), dimethylsulfoxide (DMS), and dioxane. In some embodiments, a micelle provided herein is characterized by having at least two of the aforementioned properties. In some embodiments, a micelle provided herein is characterized by having at least three of the aforementioned properties. In some embodiments, a micelle provided herein is characterized by having all of the aforementioned properties.

In certain embodiments, micelles provided herein are further or alternatively characterized by other criteria: (1) the molecular weight of the individual blocks and their relative length ratios is decreased or increased in order to govern the size of the micelle formed and its relative stability and (2) the size of the polymer cationic block that forms the shell is varied in order to provide effective complex formation with and/or charge neutralization of an anionic therapeutic agent (e.g., an oligonucleotide drug).

Moreover, in certain embodiments, micelles provided herein selectively uptake small hydrophobic molecules, such as hydrophobic small molecule compounds (e.g., hydrophobic small molecule drugs) into the hydrophobic core of the micelles. In specific embodiments, micelles provided herein selectively uptake small hydrophobic molecules, such as the hydrophobic small molecule compound pyrene into the hydrophobic core of a micelle.

### Core

Provided in certain embodiments herein, the core of a hydrophilically-shielded micelle having membrane-destabilizing copolymers described herein comprises a plurality of pH dependent membrane destabilizing hydrophobes. In certain embodiments, the core of a micelle described herein is held together at least partially, substantially, or predominantly by hydrophobic interactions.

In some embodiments, the core of a hydrophilically-shielded micelle having membrane-destabilizing copolymers described herein comprises a plurality of first chargeable species. In specific embodiments, the first chargeable species are charged or chargeable to an anionic species. It is to be understood that none, some, or all of the first chargeable species within the core are charged.

In certain embodiments, the hydrophobic block of a membrane destabilizing polymer described herein comprises a plurality of first chargeable species, and a plurality of second chargeable species. In some instances, the first chargeable species is charged or chargeable to an anionic species; and the second chargeable species is charged or chargeable to a cationic species. In some embodiments, the core of a micelle described herein comprises a plurality of first chargeable species; a plurality of second chargeable species; and a plurality of hydrophobic species.

In certain embodiments, where the core comprises a plurality of anionic chargeable species and a plurality of cationic chargeable species, the ratio of the number of the plurality of anionic chargeable species to the number of the plurality of cationic chargeable species is about 1:10 to about 10:1, about 1:8 to about 8:1, about 1:6 to about 6:1, about 1:4 to about 4:1, about 1:2 to about 2:1, about 3:2 to about 2:3, or is about 1:1. In some embodiments, the core comprises a plurality of anionic chargeable species that are anionically charged and a plurality of cationically chargeable species that is cationically charged, wherein the ratio of the number of anionically charged species to the number of cationically charged species present in the core is about 1:10 to about 10:1, about 1:8 to about 8:1, about 1:6 to about 6:1, about 1:4 to about 4:1, about 1:2 to about 2:1, about 3:2 to about 2:3, or is about 1:1.

In some embodiments, the ratio, at about a neutral pH (e.g., at a pH of about 7.4), of the number of the plurality of anionic chargeable species to the number of the plurality of cationic chargeable species is about 1:10 to about 10:1, about 1:8 to about 8:1, about 1:6 to about 6:1, about 1:4 to about 4:1, about 1:2 to about 2:1, about 2:3 to about 3:2, about 1:1.1 to about 1.1:1, or is about 1:1. In some embodiments, the core comprises a plurality of anionic chargeable species that is anionically charged and a plurality of cationically chargeable species that is cationically charged, wherein the ratio, at about a neutral pH (e.g., at a pH of about 7.4), of the number of anionically charged species to the number of cationically charged species present in the core is about 1:10 to about 10:1, about 1:8 to about 8:1, about 1:6 to about 6:1, about 1:4 to about 4:1, about 1:2 to about 2:1, about 2:3 to about 3:2, about 1:1.1 to about 1.1:1, or is about 1:1. In specific embodiments, the ratio of positively charged species present in the core to negatively charged species in the core is about 1:4 to about 4:1 at about neutral pH. In more specific embodiments, the ratio of positively charged species present in the core to negatively charged species in the core is about 1:2 to about 2:1 at about neutral pH. In specific embodiments, the ratio of positively charged species present in the core to negatively charged species in the core is about 1:1.1 to about 1.1:1 at about neutral pH.

In specific embodiments, the first chargeable species is Brønsted acid. In certain instances, as used herein, a chargeable species includes species wherein addition or removal of a proton (e.g., in a pH dependent manner), provides a cationic or anionic, respectively, species, group, or monomeric unit.

In some embodiments, the first chargeable species present in the core are species that are at least 50%, at least 60%, at least 70%, at least 80%, at least 85%, or at least 95% negatively charged at about neutral pH (e.g., at a pH of about 7.4). In specific embodiments, these first chargeable species are charged by loss of a H⁺, to an anionic species at about neutral pH. In further or alternative embodiments, the first chargeable species present in the core are species that are at least 20%, at least 30%, at least 40%, at least 50%, at least 60%, at least 70%, at least 80%, at least 85%, or at least 95% neutral or non-charged at a slightly acidic pH (e.g., a pH of about 6.5, or less; about 6.2, or less; about 6, or less; about 5.9, or less; about 5.8, or less; or about endosomal pH).

In some embodiments, the first chargeable species is, by way of non-limiting example, a carboxylic acid, anhydride, sulfonamide, sulfonic acid, sulfinic acid, sulfuric acid, phosphoric acid, phosphinic acid, boric acid, phosphorous acid, or the like.

In some embodiments, the second chargeable species present in the core are species that are at least 20%, at least 30%, at least 40%, at least 50%, at least 60%, at least 70%, at least 80%, at least 85%, or at least 95% positively charged at about neutral pH (e.g., at a pH of about 7.4). In specific embodiments, these second chargeable species are charged by addition of an H⁺, to a cationic species. In further or alternative embodiments, the second chargeable species present in the core are species that are at least 20%, at least 30%, at least 40%, at least 50%, at least 60%, at least 70%, at least 80%, at least 85%, or at least 95% positively charged at a slightly acidic pH (e.g., a pH of about 6.5, or less; about 6.2, or less; about 6, or less; about 5.9, or less; about 5.8, or less; or about endosomal pH).

In some embodiments provided herein is a hydrophilically-shielded micelle having membrane-destabilizing copolymer comprising a plurality of membrane destabilizing moieties in the core of the micelle.

### Shell

In some embodiments, the shell of a micelle described herein is hydrophilic, and includes any of the hydrophilic structures described herein, in particular a hydrophilic group that also serves as a shielding agent. In specific embodiments, the shell of a micelle described herein comprises a plurality of chargeable species. In specific embodiments, the chargeable species is charged or chargeable to a cationic species. In other specific embodiments, the chargeable species is charged or chargeable to an anionic species. In other embodiments, the shell of the micelle is hydrophilic and non-charged (e.g., substantially non-charged). It is to be understood that such hydrophilic blocks include species wherein none, some, or all of the chargeable species are charged.

In specific embodiments, the shell of a micelle described herein is polycationic at about neutral pH (e.g., at a pH of about 7.4). In some embodiments, the chargeable species in the shell of a micelle are species, groups, or monomeric units that are at least 20%, at least 30%, at least 40%, at least 50%, at least 60%, at least 70%, at least 80%, at least 85%, or at least 95% positively charged at about neutral pH (e.g., at a pH of about 7.4). In specific embodiments, these chargeable species in the shell of a micelle are charged by addition of an H⁺, to a cationic species (e.g., a Bronsted base). In further or alternative embodiments, the chargeable species in the shell of a micelle described herein are species that are at least 20%, at least 30%, at least 40%, at least 50%, at least 60%, at least 70%, at least 80%, at least 85%, or at least 95% positively charged at a slightly acidic pH (e.g., a pH of about 6.5, or less; about 6.2, or less; about 6, or less; about 5.9, or less; about 5.8, or less; or about endosomal pH).

In some embodiments, the shell of a hydrophilically-shielded micelle having membrane-destabilizing copolymers described herein is cationic at or near physiological pH (e.g., the pH of circulating human plasma). In some embodiments, the hydrophilic block is polycationic. In some embodiments, the shell comprises one or more therapeutic agents (e.g., a polynucleotide, such as siRNA), wherein the therapeutic agents are polyanionic. In some embodiments, the plurality of therapeutic agents comprise a total of x anions, and the polycationic shell of a micelle described herein comprises about 0.6 x, about 0.7·x, about 0.8 x, about 0.9 x, about 1.0 x, about 1.1 x cations, or more.

In some embodiments, the shell of a hydrophilically-shielded micelle having membrane-destabilizing copolymers described herein is hydrophilic and non-charged. Hydrophilic, non-charged species useful herein include, by way of non-limiting example, polyethylene glycol (PEG), polyethylene oxide (PEO), or the like.

In certain embodiments, the shell of a hydrophilically-shielded micelle having membrane-destabilizing copolymers described herein comprises a plurality of different hydrophilic species (e.g., at least one non-charged hydrophilic species and at least one charged hydrophilic species).

### Particle Size

In certain embodiments, the micelle provided herein is a nanoparticle having any suitable size. Size of the nanoparticles is adjusted to meet specific needs by adjusting the degree of polymerization of the core sections, shell sections, additional sections, or a combination thereof. In specific embodiments, a hydrophilically-shielded micelle having membrane-destabilizing copolymers provided herein has an average hydrodynamic diameter of about 10 nm to about 200 nm. In more specific embodiments, the micelle provided herein has an average hydrodynamic diameter of about 1 nm to about 500 nm, about 5 nm to about 250 nm, about 10 nm to about 200 nm, about 10 nm to about 100 nm, about 20 nm to about 100 nm, about 30 nm to about 80 nm, or the like in an aqueous medium. In still more specific embodiments, a hydrophilically-shielded micelle having membrane-destabilizing copolymers provided herein has an average hydrodynamic diameter of about 1 nm to about 500 nm, about 5 nm to about 250 nm, about 10 nm to about 200 nm, about 10 nm to about 100 nm, about 20 nm to about 100 nm, about 30 nm to about 80 nm, or the like in an aqueous medium with about a neutral pH (e.g., a pH of about 7.4). In some embodiments, a hydrophilically-shielded micelle having membrane-destabilizing copolymers provided herein has an average hydrodynamic diameter of about 1 nm to about 500 nm, about 5 nm to about 250 nm, about 10 nm to about 200 nm, about 10 nm to about 100 nm, about 20 nm to about 100 nm, about 30 nm to about 80 nm, or the like in human serum. In specific embodiments, provided herein is a hydrophilically-shielded micelle having membrane-destabilizing copolymers that has a particle size of about 10 nm to about 200 nm in both an aqueous medium having a pH of about 7.4 and in human serum.

### Assembly

In some embodiments, a hydrophilically-shielded micelle having membrane-destabilizing copolymers provided herein is self-assembled. In certain embodiments, the micelle is self-assembled or is capable of being self-assembled in an aqueous medium. In some embodiments, the micelle is self-assembled or is capable of being self-assembled in an aqueous medium having about neutral pH (e.g., having a pH of about 7.4). In some embodiments, the micelle is self-assembled or is capable of being self-assembled upon dilution of an organic solution of the block copolymers with an aqueous medium having about neutral pH (e.g., having a pH of about 7.4). In some embodiments, the micelle is self-assembled or is capable of being self-assembled in human serum. In some embodiments, a micelle provided herein is self-assembled.

In specific embodiments, a hydrophilically-shielded micelle having membrane-destabilizing copolymers provided herein self-assembles in an aqueous medium at least one pH value within about 6 to about 9, about 6 to about 8, about 6.5 to about 9, about 6.5 to about 8, about 6.5 to about 7.5, about 7 to about 9, or about 7 to about 8. In some embodiments, a micelle is membrane destabilizing in an aqueous medium at a pH value within about 5.0 to about 7.4. It is to be understood that as used herein, the micelles self assemble at least the pH described herein, but may also self assemble at one or more pH values outside the pH range described.

In some embodiments, a hydrophilically-shielded micelle having membrane-destabilizing copolymers provided herein self-assembles at any suitable concentration. In certain embodiments, a micelle provided herein self-assembles (e.g., has a critical assembly concentration (CAC), or the minimum concentration at which a micelle forms) of about 2 μg/mL, about 5 μg/mL, about 8 μg/mL, about 10 μg/mL, about 20 μg/mL, about 25 μg/mL, about 30 μg/mL, about 40 μg/mL, about 50 μg/mL, about 60 μg/mL, about 70 μg/mL, about 80 μg/mL, about 90 μg/mL, about 100 μg/mL, or greater. In certain embodiments, a micelle provided herein self assembles at least one concentration between about 1 μg/mL and about 100 μg/mL.

In some embodiments, the micelle (e.g., micelles) provided herein are prepared by spontaneous self-assembly of the polymers described herein. In certain embodiments, the polymers described herein assemble into the micelles provided herein upon (a) dilution of a solution of the polymer in water-miscible organic solvent into aqueous media; or (b) being dissolved directly in an aqueous solution. In some embodiments, the polymers described herein assemble into the micelles provided herein in the absence of polynucleotides.

In some embodiments, the micelles are stable to dilution in an aqueous solution. In specific embodiments, the micelles are stable to dilution at physiologic pH (including the pH of circulating blood in a human) with a critical stability concentration (e.g., a critical micelle concentration (CMC)) of approximately 50 to approximately 100 μg/mL, or approximately 10 to approximately 50 μg/mL, less than 10 μg/mL, less than 5 μg/mL, or less than 2 μg/mL. As used herein, “destabilization of a micelle” means that the polymeric chains forming a micelle at least partially disaggregate, structurally alter (e.g., expand in size and/or change shape), and/or may form amorphous supramolecular structures (e.g., non-micellic supramolecular structures). The terms critical stability concentration (CSC), critical micelle concentration (CMC), and critical assembly concentration (CAC) are used interchangeably herein.

### Stability

In some embodiments, a hydrophilically-shielded micelle having membrane-destabilizing copolymers provided herein is stable in an aqueous medium. In certain embodiments, a micelle provided herein is stable in an aqueous medium at a selected pH, e.g., about physiological pH (e.g., the pH of circulating human plasma). In specific embodiments, a micelle provided herein is stable at about a neutral pH (e.g., at a pH of about 7.4) in an aqueous medium. In specific embodiments, the aqueous medium is animal (e.g., human) serum or animal (e.g., human) plasma. In certain embodiments, a micelle provided herein is stable in human serum and/or human plasma. In specific embodiments, the micelle is stable in circulating human plasma. It is to be understood that stability of the micelle is not limited to designated pH, but that it is stable at pH values that include, at a minimum, the designated pH. In specific embodiments, a micelle described herein is substantially less stable at an acidic pH than at a pH that is about neutral. In more specific embodiments, a micelle described herein is substantially less stable at a pH of about 5.8 than at a pH of about 7.4.

In specific embodiments, the micelle is stable at a concentration of about 10 μg/mL, or greater (e.g., at about a neutral pH). In some embodiments, the micelle is stable at a concentration of about 100 μg/mL, or greater (e.g., at about a neutral pH).

### Block Copolymers

In some embodiments, block copolymers provided herein are membrane destabilizing at any suitable pH. In some embodiments, the block copolymers are membrane destabilizing (e.g., in an aqueous medium) at an endosomal pH, a pH of about 6.5, or lower, about 5.0 to about 6.5, or about 6.2, or lower.

In specific embodiments, the hydrophobic block of the block copolymers provided herein comprise a plurality of first chargeable groups, species, or monomeric units and a plurality of second chargeable species, groups, or monomeric units. In certain instances, the first chargeable groups, species or monomeric units are negatively charged or chargeable to a negative species, group, or monomeric unit. In some instances, the second chargeable groups, species, or monomeric units are positively charged or chargeable to cationic species, groups, or monomeric units. In certain embodiments, as the pH of an aqueous medium comprising a micelle described herein decreases the hydrophobic block of the block copolymers and the core of the micelle become more positively charged, resulting in a disruption of the shape and/or size of the micelle, and causing partial or substantial disruption of a membrane (e.g., an endosomal membrane surrounding the micelle).

In certain embodiments, the micelles provided herein comprise a plurality of membrane-destabilizing block copolymers which destabilize an endosomal membrane in a pH-dependent manner. In various embodiments, the membrane-destabilizing block copolymers destabilize a membrane when assembled in the micelles and/or when present independent of the micelles form (e.g., when the micelles are disassociated and/or destabilized). In some embodiments, at or near physiological pH (e.g., pH of circulating blood), the polymers making up the micelles are minimally membrane-destabilizing, but upon exposure to decreased pH (e.g., endosomal pH), the polymer is membrane-destabilizing. In certain instances, this transition to a membrane-destabilizing state occurs via the protonation of weakly acidic residues that are incorporated into the polymers, such protonation leading to an increase in the hydrophobicity of the polymers. In certain instances, the increased hydrophobicity of the polymer results in a conformational change of the micelles. In some embodiments, the mechanism of membrane destabilization of the micelles provided herein does not rely on a purely proton-sponge membrane destabilizing mechanism of polycations such as PEI or other polycations. In some embodiments, the combination of two mechanisms of membrane disruption, (a) a polycation (such as DMAEMA) and (b) a hydrophobized polyanion (such as propylacrylic acid), acting together have an additive or synergistic effect on the potency of the membrane destabilization conferred by the polymer.

In some embodiments, polymer blocks are optionally selected from, by way of non-limiting example, polynucleotides, oligonucleotides, polyethyleneglycols, hydrophilic block, hydrophobic blocks, charged blocks, or the like.

In certain embodiments, micelles described herein comprise block copolymers, wherein the block copolymers are non-peptidic and/or non-lipidic. Provided herein are micelles comprising block copolymers wherein the hydrophobic block is non-peptidic and/or non-lipidic. In certain embodiments, the micelles described herein comprise block copolymers wherein the hydrophilic block is non-peptidic and/or non-lipidic. In some embodiments, the backbone of the block copolymers forming the micelle is non-peptidic and/or non-lipidic. In certain embodiments, the backbone of the hydrophobic block is non-peptidic and/or non-lipidic. In some embodiments, the hydrophilic block is non-peptidic and/or non-lipidic. As used herein, lipids are a diverse group of compounds broadly defined as hydrophobic or amphiphilic molecules that originate entirely or in part from two distinct types of biochemical subunits: ketoacyl and isoprene groups, e.g., fatty acids, glycerolipids, glycerophoispholipids, sphingolipids, saccharolipids, polyketides, sterol lipids, and prenol lipids.

In some embodiments, provided herein is a hydrophilically-shielded micelle having membrane-destabilizing copolymers comprising a plurality of block copolymers comprising a core section (e.g., hydrophobic block) and a shell section (e.g., hydrophilic block) wherein the ratio of the number average molecular weight of the core section (e.g., hydrophobic block) to the number average molecular weight of the shell section (e.g., hydrophilic block) is present in any suitable ratio. In specific embodiments, block copolymers wherein the ratio of the number average molecular weight of the core section (e.g., hydrophobic block) to the number average molecular weight of the shell section (e.g., hydrophilic block) is present in a ratio of about 1:10 to about 5:1, about 1:1 to about 5:1, about 5:4 to about 5:1, about 1:2 to about 2:1, about 2:1, about 1.5:1, about 1.1:1, about 1.2:1, about 1.3:1, about 1.4:1, about 1.6:1, about 1.7:1, about 1.8:1, about 1.9:1, or about 2.1:1. In some embodiments, block copolymers wherein the ratio of the number average molecular weight of the core section (e.g., hydrophobic block) to the number average molecular weight of the shell section (e.g., hydrophilic block) is present in a ratio of about 2 (or more) to 1; about 1.5 (or more) to 1; about 1.1 (or more) to 1; about 1.2 (or more) to 1; about 1.3 (or more) to 1; about 1.4 (or more) to 1; about 1.6 (or more) to 1; about 1.7 (or more) to 1; about 1.8 (or more) to 1; about 1.9 (or more) to 1; or about 2.1 (or more) to 1. In specific embodiments, the ratio of the number average molecular weight of the hydrophobic block to the number average molecular weight of the hydrophilic block is about 2:1.

In specific embodiments, the micelle provided herein comprises at least one type of polymer (e.g., block copolymers and/or monoblock polymers, including monoblock copolymers) having a hydrophilic segment and a hydrophobic segment. In certain embodiments, the hydrophilic segment is a hydrophilic block and the hydrophobic segment is a hydrophobic block. In some embodiments, these polymers are non-peptidic. In other embodiments, the hydrophilic segment and the hydrophobic segment are different regions of a monoblock gradient copolymer. In various instances, a “polymeric segment” is a polymer section with a given physical property (e.g., a physical property of a block described herein, e.g., hydrophobicity, hydrophilicity, chargeability, etc.) or which comprises one or more blocks with similar physical properties (e.g., hydrophobicity, hydrophilicity, chargeability, etc.).

In certain embodiments, one or more or all of the polymers of a hydrophilically-shielded micelle having membrane-destabilizing copolymers described herein each have (1) an optionally charged hydrophilic segment (e.g., a hydrophilic block) forming at least a portion of the shell of the micelle; and (2) a substantially hydrophobic segment (e.g., a hydrophobic block) forming at least a portion of the hydrophobic core of the micelle which is stabilized through hydrophobic interactions of the core-forming polymeric segments. In some embodiments the hydrophilic segment is neutral or non-charged. In some embodiments the hydrophilic segment is charged and cationic, or polycationic. In some embodiments the hydrophilic segment is charged and anionic, or polyanionic. In some embodiments the hydrophilic segment is charged and zwitterionic. In some cases, the hydrophilic segment may serve at least three functions: (1) to form the shell of the micellic structure, (2) to increase the aqueous dispersability of the micelle, and (3) to attach to (e.g., bind) one or more therapeutic agent (e.g., oligonucleotide-based therapeutic molecules such as siRNA). In some embodiments, hydrophobic block of the block copolymers and/or core of the micelle also comprise chargeable or charged species (e.g., anionic and/or cationic species/monomeric units at a physiological pH) and are membrane-destabilizing (e.g., membrane destabilizing in a pH dependent manner). In some embodiments, the substantially hydrophobic block (e.g., hydrophobic block) and/or the core of the micelle comprises one or more chargeable species (e.g., monomeric unit, moiety, group, or the like). In more specific embodiments, the substantially hydrophobic block and/or core of the micelle comprise a plurality of cationic species and a plurality of anionic species. In still more specific embodiments, the hydrophobic block of the block copolymers and/or core of the micelle comprises a substantially similar number of cationic and anionic species (i.e., the hydrophobic block and/or core are substantially net neutral).

In certain embodiments, a hydrophilically-shielded micelle having membrane-destabilizing copolymers provided herein comprises a hydrophobic block comprising a first and a second chargeable species. In some embodiments, the first chargeable species is as described herein and the second chargeable species is chargeable to a cationic species upon protonation. In specific embodiments, the first chargeable species is non-charged at an acidic pH (e.g., an endosomal pH, a pH below about 6.5, a pH below about 6.0, a pH below about 5.8, a pH below about 5.7, or the like). In specific embodiments, the pKa of the second chargeable species is about 6 to about 10, about 6.5 to about 9, about 6.5 to about 8, about 6.5 to about 7.5, or any other suitable pKa. In certain embodiments, at least one of the first chargeable species and at least one of the second chargeable species are present on a single monomeric unit. In some embodiments, the first chargeable species is found on a first chargeable monomeric unit and the second chargeable species is on a second chargeable monomeric unit. In certain embodiments, the first chargeable species is chargeable to an anionic species upon deprotonation, the second chargeable species is chargeable to a cationic species upon protonation, and the ratio of the anionic species to the cationic species is between about 1:10 and about 10:1, about 1:6 and about 6:1, about 1:4 and about 4:1, about 1:2 and about 2:1, about 1:2 and 3:2, or about 1:1 at about a neutral pH. In some embodiments, the ratio of the first chargeable monomeric unit to the second chargeable monomeric unit is about 1:10 and about 10:1, about 1:6 and about 6:1, about 1:4 and about 4:1, about 1:2 and about 2:1, about 1:2 and 3:2, or about 1:1.

The term “copolymer”, as used herein, signifies that the polymer is the result of polymerization of two or more different monomers. A “monoblock polymer” or a “subunit polymer” of a micelle described herein is a synthetic product of a single polymerization step. The term monoblock polymer includes a copolymer (i.e. a product of polymerization of more than one type of monomers) and a homopolymer (i.e. a product of polymerization of a single type of monomers). A “block” copolymer refers to a structure comprising one or more sub-combination of constitutional or monomeric units, used interchangeably herein. Such constitutional or monomeric units comprise residues of polymerized monomers. In some embodiments, a block copolymer described herein comprises non-lipidic constitutional or monomeric units. In some embodiments, the block copolymer is a diblock copolymer. A diblock copolymer comprises two blocks; a schematic generalization of such a polymer is represented by the following: [AₐB_{b}C_{c} . . . ]ₘ-[XₓY_{y}Z_{z} . . . ]ₙ, wherein each letter stands for a constitutional or monomeric unit, and wherein each subscript to a constitutional unit represents the mole fraction of that unit in the particular block, the three dots indicate that there may be more (there may also be fewer) constitutional units in each block and m and n indicate the molecular weight of each block in the diblock copolymer. As suggested by the schematic, in some instances, the number and the nature of each constitutional unit is separately controlled for each block. The schematic is not meant and should not be construed to infer any relationship whatsoever between the number of constitutional units or the number of different types of constitutional units in each of the blocks. Nor is the schematic meant to describe any particular number or arrangement of the constitutional units within a particular block. In each block the constitutional units may be disposed in a purely random, an alternating random, a regular alternating, a regular block or a random block configuration unless expressly stated to be otherwise. A purely random configuration, for example, may have the non-limiting form: x-x-y-z-x-y-y-z-y-z-z-z . . . . A non-limiting, exemplary alternating random configuration may have the non-limiting form: x-y-x-z-y-x-y-z-y-x-z . . . , and an exemplary regular alternating configuration may have the non-limiting form: x-y-z-x-y-z-x-y-z . . . . An exemplary regular block configuration may have the following non-limiting configuration: . . . x-x-x-y-y-y-z-z-z-x-x-x . . . , while an exemplary random block configuration may have the non-limiting configuration: . . . x-x-x-z-z-x-x-y-y-y-y-z-z-z-x-x-z-z-z- . . . . In a gradient polymer, the content of one or more monomeric units increases or decreases in a gradient manner from the α end of the polymer to the ω end. In none of the preceding generic examples is the particular juxtaposition of individual constitutional units or blocks or the number of constitutional units in a block or the number of blocks meant nor should they be construed as in any manner bearing on or limiting the actual structure of block copolymers forming the micelle of this invention. In certain embodiments, provided herein is any subunit polymer or composition of subunit polymers described herein, regardless of whether or not such polymers are assembled into a micelle.

As used herein, the brackets enclosing the constitutional units are not meant and are not to be construed to mean that the constitutional units themselves form blocks. That is, the constitutional units within the square brackets may combine in any manner with the other constitutional units within the block, i.e., purely random, alternating random, regular alternating, regular block or random block configurations. The block copolymers described herein are, optionally, alternate, gradient or random block copolymers. In some embodiments, the block copolymers are dendrimer, star or graft copolymers.

In certain embodiments, block copolymers (e.g., block copolymers) of the micelles provided herein comprise ethylenically unsaturated monomers. The term “ethylenically unsaturated monomer” is defined herein as a compound having at least one carbon double or triple bond. The non-limiting examples of the ethylenically unsaturated monomers are: an alkyl (alkyl)acrylate, a methacrylate, an acrylate, an alkylacrylamide, a methacrylamide, an acrylamide, a styrene, an allylamine, an allylammonium, a diallylamine, a diallylammonium, an N-vinyl formamide, a vinyl ether, a vinyl sulfonate, an acrylic acid, a sulfobetaine, a carboxybetaine, a phosphobetaine, or maleic anhydride.

In various embodiments, any monomer suitable for providing the polymers (including, e.g., the block copolymers) of the micelles described herein is used. In some embodiments, monomers suitable for use in the preparation of the polymers (including, e.g., the block copolymers) of the micelles provided herein include, by way of non-limiting example, one or more of the following monomers: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, oligoethylene alkacrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysillpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-arylmaleimide, N-phenylmaleimide, N-alkylmaleimide, N-butylimaleimide, N-vinylpyrrolidone, N-vinylcarbazole, butadiene, isoprene, chloroprene, ethylene, propylene, 1,5-hexadienes, 1,4-hexadienes, 1,3-butadienes, 1,4-pentadienes, vinylalcohol, vinylamine, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-isopropylacrylamide, N-alkylmethacrylamides, styrene, vinylnaphthalene, vinyl pyridine, ethylvinylbenzene, aminostyrene, vinylpyridine, vinylimidazole, vinylbiphenyl, vinylanisole, vinylimidazolyl, vinylpyridinyl, vinylpolyethyleneglycol, dimethylaminomethylstyrene, trimethylammonium ethyl methacrylate, trimethylammonium ethyl acrylate, dimethylamino propylacrylamide, trimethylammonium ethylacrylate, trimethylammonium ethyl methacrylate, trimethylammonium propyl acrylamide, dodecyl acrylate, octadecyl acrylate, or octadecyl methacrylate monomers, or combinations thereof.

In some embodiments, functionalized versions of these monomers are optionally used. A functionalized monomer, as used herein, is a monomer comprising a masked or non-masked functional group, e.g. a group to which other moieties can be attached following the polymerization. The non-limiting examples of such groups are primary amino groups, carboxyls, thiols, hydroxyls, azides, and cyano groups. Several suitable masking groups are available (see, e.g., T. W. Greene & P. G. M. Wuts, Protective Groups in Organic Synthesis (2nd edition) J. Wiley & Sons, 1991. P. J. Kocienski, Protecting Groups, Georg Thieme Verlag, 1994)

Polymers described here are prepared in any suitable manner. Suitable synthetic methods used to produce the polymers provided herein include, by way of non-limiting example, cationic, anionic and free radical polymerization. In some instances, when a cationic process is used, the monomer is treated with a catalyst to initiate the polymerization. Optionally, one or more monomers are used to form a copolymer. In some embodiments, such a catalyst is an initiator, including, e.g., protonic acids (Bronsted acid) or Lewis acids, in the case of using Lewis acid some promoter such as water or alcohols are also optionally used. In some embodiments, the catalyst is, by way of non-limiting example, hydrogen iodide, perchloric acid, sulfuric acid, phosphoric acid, hydrogen fluoride, chlorosulfonic acid, methansulfonic acid, trifluoromehtanesulfonic acid, aluminum trichloride, alkyl aluminum chlorides, boron trifluoride complexes, tin tetrachloride, antimony pentachloride, zinc chloride, titanium tetrachloride, phosphorous pentachloride, phosphorus oxychloride, or chromium oxychloride. In certain embodiments, polymer synthesis is performed neat or in any suitable solvent. Suitable solvents include, but are not limited to, pentane, hexane, dichloromethane, chloroform, or dimethyl formamide (DMF). In certain embodiments, the polymer synthesis is performed at any suitable reaction temperature, including, e.g., from about −50° C. to about 100° C., or from about 0° C. to about 70° C.

In certain embodiments, the polymers are prepared by the means of a free radical polymerization. When a free radical polymerization process is used, (i) the monomer, (ii) optionally, the co-monomer, and (iii) an optional source of free radicals are provided to trigger a free radical polymerization process. In some embodiments, the source of free radicals is optional because some monomers may self-initiate upon heating at high temperature. In certain instances, after forming the polymerization mixture, the mixture is subjected to polymerization conditions. Polymerization conditions are those conditions that cause at least one monomer to form at least one polymer, as discussed herein. Such conditions are optionally varied to any suitable level and include, by way of non-limiting example, temperature, pressure, atmosphere, ratios of starting components used in the polymerization mixture and reaction time. The polymerization is carried out in any suitable manner, including, e.g., in solution, dispersion, suspension, emulsion or bulk.

In some embodiments, initiators are present in the reaction mixture. Any suitable initiators is optionally utilized if useful in the polymerization processes described herein. Such initiators include, by way of non-limiting example, one or more of alkyl peroxides, substituted alkyl peroxides, aryl peroxides, substituted aryl peroxides, acyl peroxides, alkyl hydroperoxides, substituted alkyl hydroperoxides, aryl hydroperoxides, substituted aryl hydroperoxides, heteroalkyl peroxides, substituted heteroalkyl peroxides, heteroalkyl hydroperoxides, substituted heteroalkyl hydroperoxides, heteroaryl peroxides, substituted heteroaryl peroxides, heteroaryl hydroperoxides, substituted heteroaryl hydroperoxides, alkyl peresters, substituted alkyl peresters, aryl peresters, substituted aryl peresters, or azo compounds. In specific embodiments, benzoylperoxide (BPO) and/or AIBN are used as initiators.

In some embodiments, polymerization processes are carried out in a living mode, in any suitable manner, such as but not limited to Atom Transfer Radical Polymerization (ATRP), nitroxide-mediated living free radical polymerization (NMP), ring-opening polymerization (ROP), degenerative transfer (DT), or Reversible Addition Fragmentation Transfer (RAFT). Using conventional and/or living/controlled polymerizations methods, various polymer architectures can be produced, such as but not limited to block, graft, star and gradient copolymers, whereby the monomer units are either distributed statistically or in a gradient fashion across the chain or homopolymerized in block sequence or pendant grafts. In other embodiments, polymers are synthesized by Macromolecular design via reversible addition-fragmentation chain transfer of Xanthates (MADIX) (Direct Synthesis of Double Hydrophilic Statistical Di- and Triblock Copolymers Comprised of Acrylamide and Acrylic Acid Units via the MADIX Process”, Daniel Taton, et al., Macromolecular Rapid Communications, 22, No. 18, 1497-1503 (2001).)

In certain embodiments, Reversible Addition-Fragmentation chain Transfer or RAFT is used in synthesizing ethylenic backbone polymers of this invention. RAFT is a living polymerization process. RAFT comprises a free radical degenerative chain transfer process. In some embodiments, RAFT procedures for preparing a polymer described herein employs thiocarbonylthio compounds such as, without limitation, dithioesters, dithiocarbamates, trithiocarbonates and xanthates to mediate polymerization by a reversible chain transfer mechanism. In certain instances, reaction of a polymeric radical with the C═S group of any of the preceding compounds leads to the formation of stabilized radical intermediates. Typically, these stabilized radical intermediates do not undergo the termination reactions typical of standard radical polymerization but, rather, reintroduce a radical capable of re-initiation or propagation with monomer, reforming the C═S bond in the process. In most instances, this cycle of addition to the C═S bond followed by fragmentation of the ensuing radical continues until all monomer has been consumed or the reaction is quenched. Generally, the low concentration of active radicals at any particular time limits normal termination reactions.

In some embodiments, polymers (e.g., block copolymers) utilized in the micelles provided herein have a low polydispersity index (PDI) or differences in chain length. Polydispersity index (PDI) can be determined in any suitable manner, e.g., by dividing the weight average molecular weight of the polymer chains by their number average molecular weight. The number average molecule weight is sum of individual chain molecular weights divided by the number of chains. The weight average molecular weight is proportional to the square of the molecular weight divided by the number of molecules of that molecular weight. Since the weight average molecular weight is always greater than the number average molecular weight, polydispersity is always greater than or equal to one. As the numbers come closer and closer to being the same, i.e., as the polydispersity approaches a value of one, the polymer becomes closer to being monodisperse in which every chain has exactly the same number of constitutional units. Polydispersity values approaching one are achievable using radical living polymerization. Methods of determining polydispersity, such as, but not limited to, size exclusion chromatography, dynamic light scattering, matrix-assisted laser desorption/ionization chromatography and electrospray mass chromatography are well known in the art. In some embodiments, block copolymers (e.g., membrane destabilizing block copolymers) of the micelles provided herein have a polydispersity index (PDI) of less than 2.0, or less than 1.8, or less than 1.6, or less than 1.5, or less than 1.4, or less than 1.3, or less than 1.2.

Polymerization processes described herein optionally occur in any suitable solvent or mixture thereof. Suitable solvents include water, alcohol (e.g., methanol, ethanol, n-propanol, isopropanol, butanol), tetrahydrofuran (THF) dimethyl sulfoxide (DMSO), dimethylformamide (DMF), acetone, acetonitrile, hexamethylphosphoramide, acetic acid, formic acid, hexane, cyclohexane, benzene, toluene, dioxane, methylene chloride, ether (e.g., diethyl ether), chloroform, and ethyl acetate. In one aspect, the solvent includes water, and mixtures of water and water-miscible organic solvents such as DMF.

In certain embodiments, poly(PEGMA) and other polymeric entities used herein (e.g., copolymers or copolymer blocks of BMA, DMAEMA and PAA) are prepared in any suitable manner. In one embodiment, poly(PEGMA) is prepared by polymerizing PEGMA in the presence of the RAFT CTA, ECT, and a radical initiator. In some embodiments, a block, poly(PEGMA) macroCTA is used to prepare a series of diblock copolymers where the second block contained BMA, DMAEMA and PAA. In other specific embodiments, the orientation of the blocks on the diblock polymer is reversed, such that upon self-assembly, the ω end of the polymer is exposed on the hydrophilic segment of the micelle or micelle. In various embodiments, this is achieved in any suitable manner, including a number of ways synthetically. For example, in some embodiments, the synthesis of the block copolymers described herein begins with the preparation of the PAA/BMA/DMAEMA core-forming hydrophobic block, and the shell-forming hydrophilic, charged block is added in the second synthetic step by subjecting the resulting PAA/BMA/DMAEMA macroCTA to a second RAFT polymerization step. Alternate approaches include reducing the PAA/BMA/DMAEMA macroCTA to form a thiol end and then covalently attaching a pre-formed hydrophilic, charged polymer to the formed thiol. This synthetic approach provides a method for introduction of a reactive group on the ω-end of the polymeric chain exposed to the surface of micelle thus providing alternate approaches to chemical conjugation to the micelle.

In some embodiments, block copolymers are synthesized by chemical conjugation of several polymer blocks that are prepared by separate polymerization processes.

In some instances, the block copolymers (e.g., membrane destabilizing block copolymers) comprise monomers bearing reactive groups which can be used for post-polymerization introduction of additional functionalities via know in the art chemistries, for example, “click” chemistry (for example of “click” reactions, see Wu, P.; Fokin, V. V. Catalytic Azide-Alkyne Cycloaddition: Reactivity and Applications. Aldrichim. Acta, 2007, 40, 7-17).

In specific instances, provided herein are the polymers (e.g., block copolymers including membrane destabilizing block copolymers) of the following structure:

α-[Dₛ-Xₜ]_{b}-[Bₓ-P_{y}-D_{z}]ₐ-ω [Structure 1]

α-[Bₓ-P_{y}-D_{z}]ₐ-[Dₛ-Xₜ]_{b}-ω [Structure 2]

wherein x, y, z, s and t are the mole % composition (generally, 0-50%) of the individual monomeric units D (DMAEMA), B (BMA), P (PAA), and a hydrophilic neutral monomer (X) in the polymer block, a and b are the molecular weights of the blocks, [Dₛ-Xₜ] is the hydrophilic hydrophobic block, and α and ω denote the opposite ends of the polymer. In certain embodiments, x is 50%, y is 25% and z is 25%. In certain embodiments, x is 60%, y is 20% and z is 20%. In certain embodiments, x is 70%, y is 15% and z is 15%. In certain embodiments, x is 50%, y is 25% and z is 25%. In certain embodiments, x is 33%, y is 33% and z is 33%. In certain embodiments, x is 50%, y is 20% and z is 30%. In certain embodiments, x is 20%, y is 40% and z is 40%. In certain embodiments, x is 30%, y is 40% and z is 30%. In some embodiments, a hydrophilically-shielded micelle having membrane-destabilizing copolymers described herein comprises a hydrophilic block of about 2,000 Da to about 30,000 Da, about 5,000 Da to about 20,000 Da, or about 7,000 Da to about 15,000 Da. In specific embodiments, the hydrophilic block is of about 7,000 Da, 8,000 Da, 9,000 Da, 10,000 Da, 11,000 Da, 12,000 Da, 13,000 Da, 14,000 Da, or 15,000 Da. In certain embodiments, a hydrophilically-shielded micelle having membrane-destabilizing copolymers described herein comprises a hydrophobic block of about 2,000 Da to about 50,000 Da, about 10,000 Da to about 50,000 Da, about 15,000 Da to about 35,000 Da, or about 20,000 Da to about 30,000 Da. In some specific embodiments, the polymer with a hydrophilic block is of 12,500 Da and a hydrophobic block of 25,000 Da (length ratio of 1:2) forms micelles. In some specific embodiments, the polymer with a hydrophilic block is of 10,000 Da and a hydrophobic block of 30,000 Da (length ratio of 1:3) forms micelles. In some specific embodiments, the polymer with a hydrophilic block is of 10,000 Da and a hydrophobic block of 25,000 Da (length ratio of 1:2.5) forms micelles of approximately 45 nm (as determined by dynamic light scattering measurements or electron microscopy). In some specific embodiments, the micelles are 80 or 130 nm (as determined by dynamic light scattering measurements or electron microscopy). Typically, as the molecular weight (or length) of [Dₛ-Xₜ], which forms the micelle shell, increases relative to [Bₓ-P_{y}-D_{z}] the hydrophobic block that forms the core, the size of the micelle increases. In some instances, the size of the polymer cationic block that forms the shell ([Dₛ-Xₜ] is important in providing effective complex formation/charge neutralization with the oligonucleotide drug. For example, in certain instances, for siRNA of approximately 20 base pairs (i.e., 40 anionic charges) a cationic block has a length suitable to provide effective binding, for example 40 cationic charges. For a hydrophilic block containing 80 DMAEMA monomers with a pKa value of 7.4, the block contains 40 cationic charges at pH 7.4. In some instances, stable polymer-siRNA conjugates (e.g., complexes) form by electrostatic interactions between similar numbered opposite charges. In certain instances, avoiding a large number of excess positive charge helps to prevent significant in vitro and in vivo toxicity.

In specific embodiments, the hydrophobic block of the block copolymer comprises a plurality a cationic chargeable species, for example, dimethylaminoethylmethacrylate (DMAEMA). Thus, in some embodiments, the structure of such a polymeric segment is represented by the Structure 3:

Q₁-[BMAₓ-PAA_{y}-DMAEMA_{z}]-Q₂ [Structure 3]

wherein Q₁ and Q₂ in the above designation denote other polymer blocks or end group functionalities, and wherein x, y, and z are the mole % composition (generally, 0-50%) of the individual monomeric units. In certain instances, the individual monomeric units serve individual and synergistic functions. For example, polypropyl acrylic acid, which comprises both anionic species and hydrophobic species, with a pKa value of ˜6.7 is hydrophilic above a pH of about 6.7 and is increasingly hydrophobic below a pH of about 6.7, where the carboxylates become protonated. In certain instances, increasing the hydrophobicity of the local environment, for example, by increasing the mole % of the predominantly hydrophobic monomer unit BMA in the block raises the PAA pKa and results in protonation of PAA at a higher pH, that is, the PAA containing block becomes more membrane destabilizing at a higher pH and thus more responsive to smaller acidic changes in pH below physiological pH ˜7.4. In some instances, protonation of PAA results in a large increase in hydrophobicity and subsequent conformational change to a form with membrane destabilizing properties. A third monomeric unit in the above described polymer block is the cationic species, for example DMAEMA, which, in some instances, serves multiple functions, including but not limited to the following. When matched in equivalent molar amounts to the anionic species of PAA, it creates charge neutralization and the potential for forming electrostatic interactions that can contribute to the stability of the hydrophobic core of a micelle structure where either Q₁ or Q₂ in the above structure is a hydrophilic homopolymer block, for example poly-DMAEMA.

In certain embodiments, the block copolymer (e.g., membrane destabilizing block copolymer) has the chemical Formula I:

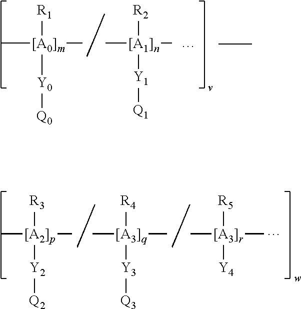

In some embodiments:
A₀, A₁, A₂, A₃ and A₄ are selected from the group consisting of —C—, —C—C—, —C(O)(C)ₐC(O)O—, —O(C)ₐC(O)— and —O(C)_{b}O—; wherein,
   a is 1-4;
b is 2-4;
Y₄ is selected from the group consisting of hydrogen, (1C-10C)alkyl, (3C-6C)cycloalkyl, O-(1C-10C)alkyl, —C(O)O(1C-10C)alkyl, C(O)NR₆(1C-10C) and aryl, any of which is optionally substituted with one or more fluorine groups;
Y₀, Y₁ and Y₂ are independently selected from the group consisting of a covalent bond, (1C-10C)alkyl-, —C(O)O(2C-10C) alkyl-, —OC(O)(1C-10C) alkyl-, —O(2C-10C)alkyl- and —S(2C-10C)alkyl- —C(O)NR₆(2C-10C) alkyl-;
Y₃ is selected from the group consisting of a covalent bond, (1C-10C)alkyl and (6C-10C)aryl; wherein
   tetravalent carbon atoms of A₁-A₄ that are not fully substituted with R₁-R₅ and Y₀-Y₄ are completed with an appropriate number of hydrogen atoms;
R₁, R₂, R₃, R₄, R₅, and R₆ are independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;
Q₀ is a residue selected from the group consisting of residues which are hydrophilic at physiologic pH, and are at least partially positively charged at physiologic pH (e.g., amino, alkylamino, ammonium, alkylammonium, guanidine, imidazolyl, pyridyl, or the like); at least partially negatively charged at physiologic pH but undergo protonation at lower pH (e.g., carboxyl, sulfonamide, boronate, phosphonate, phosphate, or the like); substantially neutral (or non-charged) at physiologic pH (e.g., hydroxy, polyoxylated alkyl, polyethylene glycol, polypropylene glycol, thiol, or the like); at least partially zwitterionic at physiologic pH (e.g., a monomeric residue comprising a phosphate group and an ammonium group at physiologic pH); conjugatable or functionalizable residues (e.g. residues that comprise a reactive group, e.g., azide, alkyne, succinimide ester, tetrafluorophenyl ester, pentafluorophenyl ester, p-nitrophenyl ester, pyridyl disulfide, or the like); or hydrogen;

Q₁ is a residue which is hydrophilic at physiologic pH, and is at least partially positively charged at physiologic pH (e.g., amino, alkylamino, ammonium, alkylammonium, guanidine, imidazolyl, pyridyl, or the like); at least partially negatively charged at physiologic pH but undergoes protonation at lower pH (e.g., carboxyl, sulfonamide, boronate, phosphonate, phosphate, or the like); substantially neutral at physiologic pH (e.g., hydroxy, polyoxylated alkyl, polyethylene glycol, polypropylene glycol, thiol, or the like); or at least partially zwitterionic at physiologic pH (e.g., comprising a phosphate group and an ammonium group at physiologic pH);
Q₂ is a residue which is positively charged at physiologic pH, including but not limited to amino, alkylamino, ammonium, alkylammonium, guanidine, imidazolyl, and pyridyl;
Q₃ is a residue which is negatively charged at physiologic pH, but undergoes protonation at lower pH, including but not limited to carboxyl, sulfonamide, boronate, phosphonate, and phosphate;
m is about 0 to less than 1.0 (e.g., 0 to about 0.49);
n is greater than 0 to about 1.0 (e.g., about 0.51 to about 1.0); wherein

m+n=1
p is about 0.1 to about 0.9 (e.g., about 0.2 to about 0.5);
q is about 0.1 to about 0.9 (e.g., about 0.2 to about 0.5); wherein:
r is 0 to about 0.8 (e.g., 0 to about 0.6); wherein

p+q+r=1
v is from about 1 to about 25 kDa, or about 5 to about 25 kDa; and,
w is from about 1 to about 50 kDa, or about 5 to about 50 kDal;
   provided that one of Q₀ or Q₁ is hydrophilic, including polyoxylated alkyl, polyethylene glycol, or polypropylene glycol.

In some embodiments, the number or ratio of monomeric residues represented by p and q are within about 30% of each other, about 20% of each other, about 10% of each other, or the like. In specific embodiments, p is substantially the same as q. In certain embodiments, at least partially charged generally includes more than a trace amount of charged species, including, e.g., at least 20% of the residues are charged, at least 30% of the residues are charged, at least 40% of the residues are charged, at least 50% of the residues are charged, at least 60% of the residues are charged, at least 70% of the residues are charged, or the like.

In certain embodiments, m is 0 and Q₁ is a residue which is hydrophilic and substantially neutral (or non-charged) at physiologic pH. In some embodiments, substantially non-charged includes, e.g., less than 5% are charged, less than 3% are charged, less than 1% are charged, or the like. In certain embodiments, m is 0 and Q₁ is a residue which is hydrophilic and at least partially cationic at physiologic pH. In certain embodiments, m is 0 and Q₁ is a residue which is hydrophilic and at least partially anionic at physiologic pH. In certain embodiments, m is >0 and n is >0 and one of and Q₀ or Q₁ is a residue which is hydrophilic and at least partially cationic at physiologic pH and the other of Q₀ or Q₁ is a residue which is hydrophilic and is substantially neutral at physiologic pH. In certain embodiments, m is >0 and n is >0 and one of and Q₀ or Q₁ is a residue which is hydrophilic and at least partially anionic at physiologic pH and the other of Q₀ or Q₁ is a residue which is hydrophilic and is substantially neutral at physiologic pH. In certain embodiments, m is >0 and n is >0 and Q₁ is a residue which is hydrophilic and at least partially cationic at physiologic pH and Q₀ is a residue which is a conjugatable or functionalizable residue. In certain embodiments, m is >0 and n is >0 and Q₁ is a residue which is hydrophilic and substantially neutral at physiologic pH and Q₀ is a residue which is a conjugatable or functionalizable residue.

In various embodiments described herein, constitutional units, that are cationic or positively charged at physiological pH (including, e.g., certain hydrophilic constitutional units) described herein comprise one or more amino groups, alkylamino groups, guanidine groups, imidazolyl groups, pyridyl groups, or the like, or the protonated, alkylated or otherwise charged forms thereof. In some embodiments, constitutional units that are cationic at normal physiological pH that are utilized herein include, by way of non-limiting example, monomeric residues of dialkylaminoalkylmethacrylates (e.g., DMAEMA). In various embodiments described herein, constitutional units, that are anionic or negatively charged at physiological pH (including, e.g., certain hydrophilic constitutional units) described herein comprise one or more acid group or conjugate base thereof, including, by way of non-limiting example, carboxylate, sulfonamide, boronate, phosphonate, phosphate, or the like. In some embodiments, constitutional units that are anionic or negatively charged at normal physiological pH that are utilized herein include, by way of non-limiting example, monomeric residues of acrylic acid, alkyl acrylic acid (e.g., methyl acrylic acid, ethyl acrylic acid, propyl acrylic acid, etc.), or the like. In various embodiments described herein, hydrophilic constitutional units that are neutral at physiologic pH comprise one or more hydrophilic group, e.g., hydroxy, polyoxylated alkyl, polyethylene glycol, polypropylene glycol, thiol, or the like. In some embodiments, hydrophilic constitutional units that are neutral at normal physiological pH that are utilized herein include, by way of non-limiting example, monomeric residues of PEGylated acrylic acid, PEGylated methacrylic acid, hydroxyalkylacrylic acid, hydroxyalkylalkacrylic acid (e.g., HPMA), or the like. In various embodiments described herein, hydrophilic constitutional units that are zwitterionic at physiologic pH comprise an anionic or negatively charged group at physiologic pH and a cationic or positively charged group at physiologic pH. In some embodiments, hydrophilic constitutional units that are zwitterionic at normal physiological pH that are utilized herein include, by way of non-limiting example, monomeric residues of comprising a phosphate group and an ammonium group at physiologic pH, such as set forth in U.S. Pat. No. 7,300,990, which is hereby incorporated herein for such disclosure, or the like.

In certain embodiments, polymers provided herein further comprise one or more constitutional unit comprising a conjugatable or functionalizable side chain (e.g., a pendant group of a monomeric residue). In some instances, a conjugatable or functionalizable side chain is a group bearing one or more reactive groups that can be used for post-polymerization introduction of additional functionalities via know in the art chemistries, for example, “click” chemistry (for example of “click” reactions, see Wu, P.; Fokin, V. V. Catalytic Azide-Alkyne Cycloaddition: Reactivity and Applications. Aldrichim. Acta, 2007, 40, 7-17). In certain embodiments, conjugatable or functionalizable side chains provided herein comprise one or more of any suitable activated group, such as but not limited to N-hydrosuccinimide (NHS)ester, HOBt (1-hydroxybenzotriazole) ester, p-nitrophenyl ester, tetrafluorophenyl ester, pentafluorophenyl ester, pyridyl disulfide group or the like.

Provided in some embodiments, a compound provided herein is a compound having the structure:

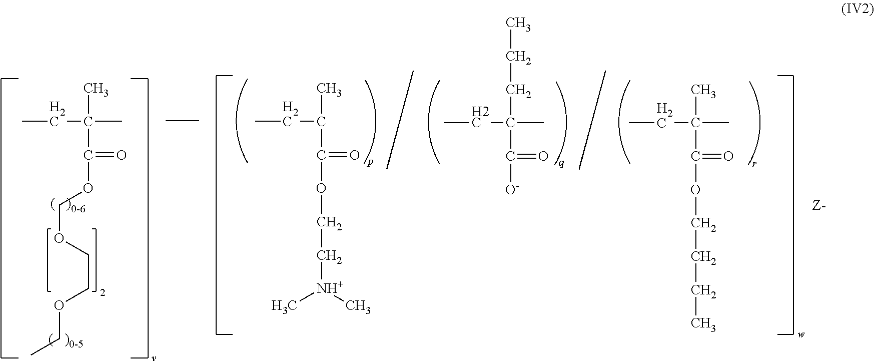

As discussed above, letters p, q and r represent the mole fraction of each constitutional unit within its block. The letters v and w represent the molecular weight (number average) of each block in the diblock copolymer.

In some embodiments, provided herein the following polymers:

[PEGMA]ᵥ-[Bₚ-/-P_{q}-/-Dᵣ]_{w} IV4

[PEGMAₘ-/-DMAEMAₙ]ᵥ-[Bₚ-/-P_{q}-/-Dᵣ]_{w} IV5

[PEGMAₘ-/-MAA(NHS)ₙ]ᵥ-[Bₚ-/-P_{q}-/-Dᵣ]_{w} IV6

[PEGMAₘ-/-PDSMₙ]ᵥ-[Bₚ-/-P_{q}-/-Dᵣ]_{w} IV9

In some embodiments, B is butyl methacrylate residue; P is propyl acrylic acid residue; D and DMAEMA are dimethylaminoethyl methacrylate residue; PEGMA is polyethyleneglycol methacrylate residue (e.g., with 1-20 ethylene oxide units, such as illustrated in compound IV2, or 4-5 ethylene oxide units, or 7-8 ethylene oxide units); MAA(NHS) is methylacrylic acid-N-hydroxy succinamide residue; HPMA is N-(2-hydroxypropyl)methacrylamide residue; and PDSM is pyridyl disulfide methacrylate residue. In certain embodiments, the terms m, n, p, q, r, w and v are as described herein. In specific embodiments, w is about 1× to about 5×v.

Compounds of Formulas IV4, IV5, IV6, and IV9 are examples of polymers provided herein comprising a variety of constitutional unit(s) making up the first block of the polymer. In some embodiments, the constitutional unit(s) of the first block are varied or chemically treated in order to create polymers where the first block is or comprises a constitutional unit that is neutral (e.g., PEGMA), cationic (e.g., DMAEMA), anionic (e.g., PEGMA-NHS, where the NHS is hydrolyzed to the acid, or acrylic acid), ampholytic (e.g., DMAEMA-NHS, where the NHS is hydrolyzed to the acid), or zwiterrionic (for example, poly[2-methacryloyloxy-2′ trimethylammoniumethyl phosphate]). In some embodiments, polymers comprising pyridyl disulfide functionality in the first block, e.g., [PEGMA-PDSM]-[B-P-D], that can be and is optionally reacted with a thiolated siRNA to form a polymer-siRNA conjugate.

### Polymerizable Hydrophilic Monomers

In some embodiments, a block copolymer described herein comprises one or more hydrophilic polymerizable constitutional units in the hydrophilic block. Provided in this section (and also throughout this description) are examples of hydrophilic monomers that are used as components of the hydrophilic block of the copolymers described herein.

In certain embodiments, hydrophilic polymerizable monomeric units comprise ethylenically unsaturated monomers. The term “ethylenically unsaturated monomer” is defined herein as a compound having at least one carbon double or triple bond. Non-limiting examples of ethylenically unsaturated monomers include an alkyl (alkyl)acrylate, a alkyl methacrylate, an alkylacrylic acid, an N-alkylacrylamide, a methacrylamide, a styrene, an allylamine, an allylammonium, a diallylamine, a diallylammonium, an N-vinyl formamide, a vinyl ether, a vinyl sulfonate, an acrylic acid, a sulfobetaine, a carboxybetaine, a phosphobetaine, or maleic anhydride.

In some embodiments, a hydrophilic ethylenically unsaturated polymerizable monomer is a vinylic monomer. In some embodiments, a hydrophilic ethylenically unsaturated polymerizable monomer is an acrylic monomer of formula:

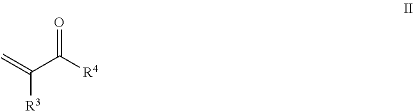

wherein
R³ is hydrogen, halogen, hydroxyl, or optionally substituted C₁-C₃ alkyl;
R⁴ is —SR⁵, —OR⁵, —NR⁶R⁷, or
R⁴ is a polyoxylated alkyl, optionally substituted by hydroxyl, thiol, —NR⁹R¹⁰, a cleavable moiety or a functionalizable moiety;
R⁵ is a polyoxylated alkyl, optionally substituted by hydroxyl, thiol, —NR⁹R¹⁰, a cleavable group or a functionalizable group;
R⁶ and R⁷ are each independently H or polyoxylated alkyl, optionally substituted by hydroxyl, thiol, —NR⁹R¹⁰, a cleavable group or a functionalizable group, provided that R⁶ and R⁷ are not both H; or
R⁶ and R⁷ together with the Nitrogen to which they are attached form an optionally substituted heterocycle;
R⁹ and R¹⁰ are each independently H or C₁-C₆ alkyl; or
R⁹ and R¹⁰ together with the nitrogen to which they are attached form a heterocycle.

In some embodiments the polyoxylated alkyl is selected from a polyethylene glycol group, a polypropylene glycol group, including optionally substituted groups thereof.

In some embodiments, a polymerizable acrylic monomer is an optionally substituted acrylic acid, an optionally substituted acrylate or an optionally substituted acrylamide.

In some embodiments, the functionalizable moiety is suitable for forming a covalent bond to a therapeutic agent (including an siRNA) or a targeting moiety. In one embodiment, the functionalizable moiety is NHS.

In some embodiments, a hydrophilic polymerizable monomer is cationic (e.g., R³ and/or R⁴ comprises a deprotonable cationic species). In some of such embodiments, a deprotonable cationic species is an acyclic amine, acyclic imine, cyclic amine, cyclic imine, amino groups, alkylamino groups, guanidine groups, imidazolyl groups, pyridyl groups, triazolyl groups or the like or combinations thereof.

In some embodiments, polymerizable hydrophilic constitutional units that are cationic at normal physiological pH that are utilized herein include, by way of non-limiting example, monomeric residues of dialkylaminoalkylmethacrylates (e.g., DMAEMA).

In some embodiments, a hydrophilic polymerizable monomer is anionic (e.g., R³ and/or R⁴ comprises a protonable anionic species). In some of such embodiments, a protonable anionic species is a carboxylic acid, sulfonamide, boronic acid, sulfonic acid, sulfinic acid, sulfuric acid, phosphoric acid, phosphinic acid, or combinations thereof. In some embodiments, polymerizable constitutional units that are anionic at normal physiological pH that are utilized herein include, by way of non-limiting example, monomeric residues derived from polymerization of a (C₂-C₈) alkylacrylic acid.

In some embodiments, a hydrophilic polymerizable monomeric unit comprises a cleavable moiety that is a removable protecting group (e.g., an ester protecting group). In some embodiments, a cleavable moiety is a group that is hydrolysed under physiological conditions (e.g., in the presence of a protease).

In some embodiments, hydrophilic polymerizable monomeric units comprise conjugatable or functionalizable moieties (e.g. monomers that comprise a reactive group, e.g., azide, alkyne, succinimide ester, tetrafluorophenyl ester, pentafluorophenyl ester, p-nitrophenyl ester, pyridyl disulfide, or the like);

In some embodiments, a functionalizable group is a reactive group that allows for covalent association between a micellic assembly (including the components thereof) and a therapeutic agent (e.g., an oligonucleotide or siRNA or peptide). Such covalent association is achieved through any suitable chemical conjugation method, including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In some embodiments, conjugation is also performed with pH-sensitive bonds and linkers, including, but not limited to, hydrazone and acetal linkages. Any other suitable conjugation method is optionally utilized as well, for example a large variety of conjugation chemistries

In some embodiments, any hydrophilic monomeric unit described herein comprises partially positively charged species at physiologic pH (e.g., amino, alkylamino, ammonium, alkylammonium, guanidine, imidazolyl, pyridyl, or the like); at least partially negatively charged species at physiologic pH that undergo protonation at lower pH (e.g., carboxyl, sulfonamide, boronate, phosphonate, phosphate, or the like); substantially neutral (or non-charged) species at physiologic pH (e.g., hydroxy, polyoxylated alkyl, polyethylene glycol, polypropylene glycol, thiol, or the like); at least partially zwitterionic species at physiologic pH (e.g., a monomeric residue comprising a phosphate group and an ammonium group at physiologic pH); conjugatable or functionalizable residues (e.g. residues that comprise a reactive group, e.g., azide, alkyne, succinimide ester, tetrafluorophenyl ester, pentafluorophenyl ester, p-nitrophenyl ester, pyridyl disulfide, or the like); or hydrogen.

In some embodiments, a constitutional unit derived from a hydrophilic polymerizable monomer of formula II is of formula III:

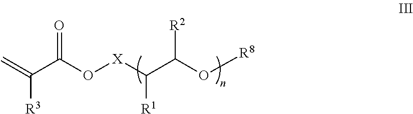

Wherein
X is absent or optionally substituted C₁-C₃ alkyl;
R¹, R² and R³ are each independently hydrogen, halogen, C₁-C₃ fluoroalkyl or optionally substituted C₁-C₃ alkyl;
n is an integer ranging from 2 to 20,
R⁸ is hydrogen, C₁-C₆ alkyl, C₁-C₆ alkenyl, C₁-C₆ alkynyl, cycloalkyl, heterocycloalkyl, aryl, or heteroaryl optionally substituted by hydroxyl, thiol, —NR⁹R¹⁰, a cleavable group or a functionalizable group; R⁹ and R¹⁰ are each independently H or C₁-C₆ alkyl; or
R⁹ and R¹⁰ together with the nitrogen to which they are attached form a heterocycle.

In some embodiments, the functionalizable moiety is suitable for forming a covalent bond to a therapeutic agent (including an siRNA) or a targeting moiety. In one embodiment, the functionalizable moiety is NHS.

In some embodiments, a constitutional unit derived from a hydrophilic polymerizable monomer of formula II is of Formula IV:

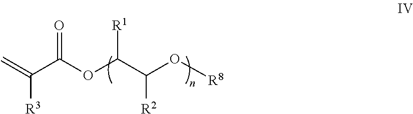

Wherein
R¹, R² and R³ are each independently hydrogen, halogen, C₁-C₃ fluoroalkyl or optionally substituted C₁-C₃ alkyl;
n is an integer ranging from 2 to 20,
R⁸ is hydrogen, C₁-C₆ alkyl, C₁-C₆ alkenyl, C₁-C₆ alkynyl, cycloalkyl, heterocycloalkyl, aryl, or heteroaryl optionally substituted by hydroxyl, thiol, —NR⁹R¹⁰, a cleavable group or a functionalizable group;
R⁹ and R¹⁰ are each independently H or C₁-C₆ alkyl; or
R⁹ and R¹⁰ together with the nitrogen to which they are attached form a heterocycle.

In some embodiments, the functionalizable moiety is suitable for forming a covalent bond to a therapeutic agent (including an siRNA) or a targeting moiety. In one embodiment, the functionalizable moiety is NHS.

In some embodiments, the hydrophilic block of a block copolymer described herein comprises hydrophilic polymerizable monomers wherein at least 10%, at least 25%, at least 40%, at least 55% or at least 70% by weight of the constitutional units comprise a monomer of Formula II, III or IV. In some embodiments, the hydrophilic block of a block copolymer described herein comprises hydrophilic polymerizable monomers wherein at least 10%, at least 25%, at least 40%, at least 55% or at least 70% by weight of the constitutional units comprise a monomer of Formula II, III or IV and wherein n is from 5 to 12.

In some embodiments, the hydrophilic block of a block copolymer described herein comprises hydrophilic polymerizable monomers wherein at least 10%, at least 25%, at least 40%, at least 55% or at least 70% by weight of the constitutional units comprise a hydrophilic polymerizable monomer with a pendant group attached thereto.

### Hydrophobic Block

Provided in certain embodiments herein, the hydrophobic block is a membrane destabilizing block copolymer that is or comprises a pH dependent membrane destabilizing hydrophobe.

In some embodiments, the block copolymer described herein comprises a first species that is anionic at about neutral pH. In certain embodiments, the block copolymer described herein comprises a first species that is anionic at about neutral pH, the hydrophobic block being a copolymer block. In some embodiments, the block copolymer described herein comprises a first species that is anionic at about neutral pH, the first species being hydrophobically shielded (e.g., by being in proximity of the polymer backbone of a polymer block comprising pendant hydrophobic moieties). In certain embodiments, the block copolymer described herein comprises a first species that is anionic at about neutral pH and a second chargeable species that is cationic at about neutral pH.

In certain embodiments, the membrane destabilizing polymer described herein comprises at least one first species, group, or monomeric unit, and at least one second species, group, or monomeric unit. In some instances, the first species, group, or monomeric unit is as described above and the second species, group, or monomeric unit is charged or chargeable to a cationic species, group, or monomeric unit. In some embodiments, the membrane destabilizing polymer described herein comprises at least one first species, group, or monomeric unit; at least one second species, group, or monomeric unit; and at least one additional species, group, or monomeric unit. In specific embodiments, the additional species, group, or monomeric unit is a neutral species, group, or monomeric unit. In certain embodiments, the additional species, group, or monomeric unit is a hydrophobic species, group, or monomeric unit.

In certain embodiments, where the hydrophobic block comprises at least one anionic species, group, or monomeric unit and at least one cationic species, group, or monomeric unit, the ratio of the number of the at least one anionic species, group, or monomeric unit to the number of the at least one cationic species, group or monomeric unit is about 1:10 to about 10:1, about 1:8 to about 8:1, about 1:6 to about 6:1, about 1:4 to about 4:1, about 1:2 to about 2:1, about 3:2 to about 2:3, or is about 1:1. In some embodiments, the hydrophobic block comprises at least one anionic species, group, or monomeric unit that is anionically charged and at least one cationic species, group, or monomeric unit that is cationically charged, wherein the ratio of the number of anionically charged species, group, or monomeric unit to the number of cationically charged species, group, or monomeric unit present on the hydrophobic block is about 1:10 to about 10:1, about 1:8 to about 8:1, about 1:6 to about 6:1, about 1:4 to about 4:1, about 1:2 to about 2:1, about 3:2 to about 2:3, or is about 1:1.

In some embodiments, the first chargeable species, groups, or monomeric units present in the hydrophobic block are species, groups, or monomeric units that are at least 50%, at least 60%, at least 70%, at least 80%, at least 85%, or at least 95% negatively charged at about neutral pH (e.g., at a pH of about 7.4). In specific embodiments, these first chargeable species, groups, or monomeric units are charged by loss of an H⁺, to an anionic species at about neutral pH. In further or alternative embodiments, the first chargeable species, groups, or monomeric units present in the hydrophobic block are species, groups, or monomeric units that are at least 20%, at least 30%, at least 40%, at least 50%, at least 60%, at least 70%, at least 80%, at least 85%, or at least 95% neutral or non-charged at a slightly acidic pH (e.g., a pH of about 6.5, or less; about 6.2, or less; about 6, or less; about 5.9, or less; about 5.8, or less; or about endosomal pH).

In some embodiments, the first species or group is, by way of non-limiting example, a carboxylic acid, anhydride, sulfonamide, sulfonic acid, sulfinic acid, sulfuric acid, phosphoric acid, phosphinic acid, boric acid, phosphorous acid, or the like. Similarly, in certain embodiments, a first monomeric unit useful herein is a monomeric unit that comprises a carboxylic acid, anhydride, sulfonamide, sulfonic acid, sulfinic acid, sulfuric acid, phosphoric acid, phosphinic acid, boric acid, phosphorous acid, or the like. In specific embodiments, a first monomeric unit useful herein is a (C₂-C₈)alkylacrylic acid.

In some embodiments, the anionic species is any organic or inorganic acid residue that is optionally present, either as a protected species, e.g., an ester, or as the free acid, in the selected polymerization process. In some embodiments, the anionic species is a weak acid, such as but not limited to the following groups: boronic acid, sulfonamide, phosphonic acid, arsonic acid, phosphinic acid, phosphate, carboxylic acid, xanthenes, tetrazole or their derivatives (e.g. esters). In certain embodiments monomers such as maleic-anhydride, (Scott M. Henry, Mohamed E. H. El-Sayed, Christopher M. Pirie, Allan S. Hoffman, and Patrick S. Stayton pH-Responsive Poly(styrene-alt-maleic anhydride) Alkylamide Copolymers for Intracellular Drug Delivery. Biomacromolecules 2006, 7, 2407-2414) are used for introduction of first chargeable species by post-polymerization hydrolysis of the maleic anhydride monomeric units. In specific embodiments, a species that is anionic at normal physiological pH includes carboxylic acids such as, but not limited to, 2-propyl acrylic acid or, more accurately, the constitutional unit derived from it, 2-propylpropionic acid, —CH₂C((CH₂)₂CH₃)(COOH) (PAA).

In some embodiments, the second species, groups, or monomeric units present in the hydrophobic block are species, groups, or monomeric units that are at least 20%, at least 30%, at least 40%, at least 50%, at least 60%, at least 70%, at least 80%, at least 85%, or at least 95% positively charged at about neutral pH (e.g., at a pH of about 7.4). In specific embodiments, these second species, groups, or monomeric units are charged by addition of an H⁺, to a cationic species. In further or alternative embodiments, the second species, groups, or monomeric units present in the hydrophobic block are species, groups, or monomeric units that are at least 20%, at least 30%, at least 40%, at least 50%, at least 60%, at least 70%, at least 80%, at least 85%, or at least 95% positively charged at a slightly acidic pH (e.g., a pH of about 6.5, or less; about 6.2, or less; about 6, or less; about 5.9, or less; about 5.8, or less; or about endosomal pH).

In specific embodiments, the second monomeric unit is a Bronsted base. In certain embodiments, the second species or group is an amine (including, e.g., non-cyclic and cyclic amines). In some embodiments, the second monomeric unit is a monomeric unit comprising an amine, such as, by way of non-limiting example, N,N-di(C₁-C₆)alkyl-amino(C₁-C₆)alkyl-ethacrylate, N,N-di(C₁-C₆)alkyl-amino(C₁-C₆)alkyl-methacrylate, or N,N-di(C₁-C₆)alkyl-amino(C₁-C₆)alkyl-acrylate. In some embodiments, the second monomeric unit comprises a nitrogen heterocycle, e.g. an imidazole, a pyridine, a piperidine, a pyrimidine, or the like.

In some embodiments, the second species is cationic. In certain embodiments, the second species is cationic at physiological pH. In specific embodiments, cationic at physiological pH species are nitrogen species such as ammonium, —NRR′R″, guanidinium (—NRC(═NR′H)⁺NR″R′″, including canonical forms), wherein the R groups are independently hydrogen, alkyl, cycloalkyl or aryl or two R groups bonded to the same or adjacent nitrogen atoms may be also be joined to one another to form a heterocyclic species such as but not limited to pyrrole, imidazole, pyrimidine, or indole.

In some embodiments, the either the first or second species is present in a zwitterionic monomeric units (i.e., wherein an anionic and a cationic chargeable species are present in the same monomeric unit).

In certain embodiments, the hydrophobic block comprises at least one non-charged or neutral monomeric unit, group, or species. In some embodiments, the non-chargeable monomeric unit is hydrophobic or comprises a hydrophobic group or species. In certain embodiments, the hydrophobic group has a π value of about 1, or more; about 2, or more; about 3, or more; about 4, or more; about 5, or more; or the like. In specific embodiments, the non-chargeable monomeric unit is, by way of non-limiting example, a (C₂-C₈)alkyl-ethacrylate, a (C₂-C₈)alkyl-methacrylate, or a (C₂-C₈)alkyl-acrylate.

In some embodiments, the block copolymers in the hydrophobic block comprise a plurality of hydrophobic species. In some embodiments, the block copolymer comprises hydrophobic monomeric units. In certain embodiments, the hydrophobic monomeric unit is a vinyl substituted aromatic or heteroaromatic compound. In further specific embodiments, hydrophobic monomers are alkyl (alkyl)acrylates. In specific embodiments, the hydrophobic monomer is a styrene derivative.

In some embodiments, provided herein the block copolymer has a number average molecular weight (Mn) of about 2,000 dalton to about 150,000,000 dalton; 2,000 dalton to about 100,000 dalton; about 5,000 dalton to about 100,000 dalton; about 5,000 dalton to about 50,000 dalton; or about 10,000 dalton to about 50,000 dalton.

### Hydrophilic Block

In certain embodiments, the micelles described herein comprise one or more shielding agents. In some embodiments, the polynucleotide carrier block/segment comprises a PEG substituted monomeric unit (e.g., the PEG is a side chain or a pendant chain and does not comprise the backbone of the polynucleotide carrier block). In some instances, one or more of the polymers (e.g., block copolymers) utilized in the micelles described herein comprise polyethylene glycol oligomer or polymer chains (PEG) with molecular weights of approximately from 100 to approximately 2,000. In some embodiments, PEG chains are attached to polymer ends groups, or to one or more pendant modifiable group present in a polymer of a micelle provided herein. In certain embodiments, a monomer comprising a PEG residue of 2-20 ethylene oxide units is co-polymerized to form the hydrophilic portion of the polymer forming a micelle provided herein.

In some instances a shielding agent enhances the stability of the therapeutic agent (e.g., polynucleotide or peptide, etc.) against enzymatic digestion in plasma. In some instances, a shielding agent reduces toxicity of micelles described herein (e.g., block copolymer attached to polynucleotides). In some embodiments, a shielding agent comprises a plurality of neutral hydrophilic monomeric residues. In some instances, a shielding polymer is covalently coupled to a membrane destabilizing block copolymer through an end group of the polymer. In some embodiments, a shielding agent is a covalently coupled pendant moiety attached to one or more monomeric residues of the polymer. In some embodiments, a plurality of monomeric residues in a micelle described herein comprise pendant shielding species (e.g., a polyethylene glycol (PEG) oligomer (e.g., having 20 or less repeat units) or polymer (e.g., having more than 20 repeat units)) covalently coupled through a functional group to the polyethylene glycol oligomer or polymer. In some instances, a block copolymer comprises a polyethylene glycol (PEG) oligomer or polymer covalently coupled to the alpha end or the omega end of the membrane destabilizing block of the copolymer.

In certain embodiments, the polynucleotide carrier block/segment comprises a monomeric unit that serves to shield, at least in part, the charge (e.g., cationic charges) on the polynucleotide carrier block/segment. In particular embodiments, the shielding arises, at least in part, form a pendant moiety on the monomeric unit that comprises, at least part, of the polynucleotide carrier block/segment. Such shielding optionally lowers the cellular toxicity from excessive charges in this segment.

In some embodiments, the hydrophilic block is non-charged at an approximately physiological pH, e.g. pH 7.4. In particular embodiments, the hydrophilic block includes a constitutional unit that has a hydrophilic pendant group, the constitutional unit originating as a polymerizable monomeric unit with the same hydrophilic pendant group, or a polymerizable monomeric unit with a different hydrophilic pendant group (e.g., the different hydrophilic pendant group on the polymerizable monomeric unit has a protecting group that is removed after the monomeric unit has been incorporated into the hydrophilic polymer block). In any case, the hydrophilic pendant group on the polymerizable monomer and the hydrophilic pendant group on the constitutional unit of the hydrophilic block both share the following structural feature:

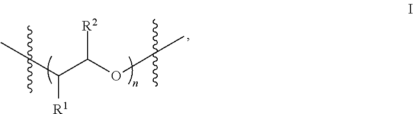

where R¹ and R² are each independently selected from the group consisting of hydrogen, halogen, C₁-C₃ haloalkyl, and optionally substituted C₁-C₃ alkyl, and n is an integer ranging from 2 to 20. Such a non-charged hydrophilic block optionally includes other constitutional units with non-charged pendant groups, including hydrophilic, hydrophobic or hydro-agnostic pendant groups, or zwitterionic (charge-balanced) groups; provided that the overall character of the block remains hydrophilic.

In specific embodiments, the hydrophilic block is non-charged at about neutral pH (e.g., at a pH of about 7.4). In specific embodiments, the hydrophilic block is also non-charged at about endosomal pH.

In some embodiments, the hydrophilic block is charged (cationic or anionic) at an approximately physiological pH, e.g. pH 7.4. In particular embodiments, the hydrophilic block includes a constitutional unit that has a hydrophilic pendant group, the constitutional unit originating as a polymerizable monomeric unit with the same hydrophilic pendant group, or a polymerizable monomeric unit with a different hydrophilic pendant group (e.g., the different hydrophilic pendant group on the polymerizable monomeric unit has a protecting group that is removed after the monomeric unit has been incorporated into the hydrophilic polymer block). In any case, the hydrophilic pendant group on the polymerizable monomer and the hydrophilic pendant group on the constitutional unit of the hydrophilic block both share the following structural feature:

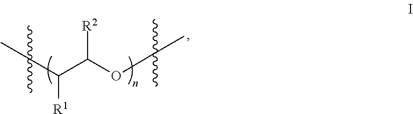

where R¹ and R² are each independently selected from the group consisting of hydrogen, halogen, C₁-C₃ haloalkyl, and optionally substituted C₁-C₃ alkyl, and n is an integer ranging from 2 to 20.

In some embodiments, the charged hydrophilic block further comprises at least one hydrophilic (e.g., non-charged, cationic, anionic, or zwitterionic) species, group, or monomeric unit. In specific embodiments, the hydrophilic block comprises at least one chargeable species, group, or monomeric unit. In specific embodiments, the chargeable species, group, or monomeric unit is charged or chargeable to a cationic species, group, or monomeric. In other specific embodiments, the chargeable species, group, or monomeric unit is charged or chargeable to an anionic species, group, or monomeric unit. In specific embodiments, the chargeable species, group, or monomeric unit is charged or chargeable to a zwitterionic species, group, or monomeric. It is to be understood that such hydrophilic blocks include species, groups, and/or monomeric units wherein none, some, or all of the chargeable species, groups, or monomeric units are charged.

In specific embodiments, the hydrophilic block is polycationic at about neutral pH (e.g., at a pH of about 7.4). In specific embodiments, the hydrophilic block is also polycationic at about endosomal pH

In specific embodiments, the hydrophilic block is polyanionic at about neutral pH (e.g., at a pH of about 7.4). In specific embodiments, the hydrophilic block is also polyanionic at about endosomal pH

In specific embodiments, the hydrophilic block is zwitterionic at about neutral pH (e.g., at a pH of about 7.4). In specific embodiments, the hydrophilic block is also zwitterionic at about endosomal pH

In some embodiments, the chargeable species, groups, or monomeric units present in the charged hydrophilic block are species, groups, or monomeric units that are at least 20%, at least 30%, at least 40%, at least 50%, at least 60%, at least 70%, at least 80%, at least 85%, or at least 95% positively charged at about neutral pH (e.g., at a pH of about 7.4). In specific embodiments, these chargeable species, groups, or monomeric units in the charged hydrophilic block are charged by addition of an H⁺, to a cationic species. In further or alternative embodiments, the chargeable species, groups, or monomeric units in the charged hydrophilic block are species, groups, or monomeric units that are at least 20%, at least 30%, at least 40%, at least 50%, at least 60%, at least 70%, at least 80%, at least 85%, or at least 95% positively charged at a slightly acidic pH (e.g., a pH of about 6.5, or less; about 6.2, or less; about 6, or less; about 5.9, or less; about 5.8, or less; or about endosomal pH).

In specific embodiments, cationic species at physiological pH species are nitrogen species such as ammonium, —NRR′R″, guanidinium (—NRC(═NR′H)⁺NR″R′″, including canonical forms) wherein the R groups are independently hydrogen, alkyl, cycloalkyl or aryl or two R groups bonded to the same or adjacent nitrogen atoms may be also be joined to one another to form a heterocyclic species such as but not limited to pyrrole, imidazole, or indole.

In some embodiments, the anionic chargeable species is any organic or inorganic acid residue that is optionally present, either as a protected species, e.g., an ester, or as the free acid, in the selected polymerization process. In some embodiments, the anionic chargeable species is a weak acid, such as but not limited to the following groups: boronic acid, sulfonamide, phosphonic acid, arsonic acid, phosphinic acid, phosphate, carboxylic acid, xanthenes, tetrazole or their derivatives (e.g. esters). In certain embodiments monomers such as maleic-anhydride, (Scott M. Henry, Mohamed E. H. El-Sayed, Christopher M. Pirie, Allan S. Hoffman, and Patrick S. Stayton pH-Responsive Poly(styrene-alt-maleic anhydride) Alkylamide Copolymers for Intracellular Drug Delivery. Biomacromolecules 2006, 7, 2407-2414) are used for introduction of first chargeable species by post-polymerization hydrolysis of the maleic anhydride monomeric units. In specific embodiments, a chargeable species that are anionic at normal physiological pH are carboxylic acids such as, but not limited to, 2-propyl acrylic acid or, more accurately, the constitutional unit derived from it, 2-propylpropionic acid, —CH₂C((CH₂)₂CH₃)(COOH) (PAA).

In specific embodiments, the chargeable monomeric unit of the hydrophilic block is a Bronsted base. In certain embodiments, the chargeable species or group of the hydrophilic block is an amine (including, e.g., non-cyclic and cyclic amines). In some embodiments, the chargeable monomeric unit of the hydrophilic block is a monomeric unit comprising an amine, such as, by way of non-limiting example, N,N-di(C₁-C₆)alkyl-amino(C₁-C₆)alkyl-ethacrylate, N,N-di(C₁-C₆)alkyl-amino(C₁-C₆)alkyl-methacrylate, or N,N-di(C₁-C₆)alkyl-amino(C₁-C₆)alkyl-acrylate. In some embodiments, the chargeable monomeric unit of the hydrophilic block is a monomeric unit comprising a nitrogen heterocycle, e.g., an imidazole or pyridine.

In some embodiments, the hydrophilic block is attached to a therapeutic agent (e.g., a polynucleotide, such as siRNA) which is a polyanion.

In some embodiments, provided herein the hydrophilic block has a number average molecular weight (Mn) of about 1,000 dalton to about 100,000 dalton; 1,000 dalton to about 100,000 dalton; about 3,000 dalton to about 100,000 dalton; about 5,000 dalton to about 50,000 dalton; about 5,000 dalton to about 25,000 dalton; or about 5,000 dalton to about 20,000 dalton.

In specific embodiments, the hydrophilic block is non-charged and hydrophilic at about neutral pH (e.g., at a pH of about 7.4). In certain embodiments, the hydrophilic block is free or substantially free of chargeable groups. In some embodiments, a non-charged hydrophilic block comprises or is polyethylene glycol (PEG), polyethylene oxide (PEO) or the like.

In certain embodiments, the hydrophilic block comprises a functionalizing group (e.g., a solubilizing group). In specific embodiments, the functionalizing group is a polyethylene glycol (PEG) group. In certain embodiments, the hydrophilic block comprises a polyethylene glycol (PEG) groups, chains or blocks with molecular weights of approximately from 1,000 to approximately 30,000. In some embodiments, the PEG is a part of (e.g., incorporated into) the hydrophilic block chain. In certain embodiments, the PEG is incorporated into the hydrophilic block chain during polymerization.

In certain embodiments, provided herein are micelles comprising a first membrane destabilizing block copolymer with a polycationic hydrophilic block, and a second membrane destabilizing block copolymer with a PEG hydrophilic block. In certain embodiments, one or more monomeric units of the hydrophilic block are substituted or functionalized with a PEG group. In some embodiments, PEG is conjugated to block copolymer ends groups, or to one or more pendant modifiable group present in a micelle provided herein. In some embodiments, PEG residues are conjugated to modifiable groups within the hydrophilic segment or block (e.g., a hydrophilic block) of a polymer (e.g., block copolymer) of a micelle provided herein. In certain embodiments, a monomer comprising a PEG residue is co-polymerized to form the hydrophilic portion of the polymer forming the micelle provided herein

### Therapeutic Agents

Provided in certain embodiments herein is a hydrophilically-shielded micelle having membrane-destabilizing copolymers comprising at least one research reagent, at least one diagnostic agent, at least one therapeutic agent, or a combination thereof. In some embodiments, such therapeutic agents are present in the shell of the micelle, in the core of the micelle, on the surface of the micelle, or a combination thereof. In specific embodiments, the therapeutic agent is a polynucleotide that is not in the core of the micelle.

In various embodiments, research reagents, diagnostic agents, and/or therapeutic agents are attached to the micelle or block copolymers thereof in any suitable manner. In specific embodiments, attachment is achieved through covalent bonds, non-covalent interactions, static interactions, hydrophobic interactions, or the like, or combinations thereof. In some embodiments, the research reagents, diagnostic agents, and/or therapeutic agents are attached to a hydrophilic block of block copolymers. In certain embodiments, the research reagents, diagnostic agents, or therapeutic agents form the hydrophilic block of a block copolymer. In some embodiments, the research reagents, diagnostic agents, or therapeutic agents are in the shell of the micelle.

In some embodiments, provided herein is a hydrophilically-shielded micelle having membrane-destabilizing copolymers comprising a first therapeutic agent in the shell of the micelle and a second therapeutic agent in the core of the micelle. In specific embodiments, the first therapeutic agent is a polynucleotide. And the second therapeutic agent is a hydrophobic drug. In certain embodiments, provided herein is a micelle comprising a hydrophobic drug (e.g., small molecule hydrophobic drug) in the core of the micelle.

In certain embodiments, provided herein is a hydrophilically-shielded micelle having membrane-destabilizing copolymers comprising at least 1-5, 5-250, 5-1000, 250-1000, at least 2, at least 5, at least 10, at least 20, or at least 50 therapeutic agents. In some embodiments, provided herein is a composition comprising a plurality of micelles described herein, wherein the micelles therein comprise, on average, at least 1-5, 5-250, 5-1000, 250-1000, at least 2, at least 5, at least 10, at least 20, or at least 50 therapeutic agents.

In some embodiments, therapeutic agents, diagnostic agents, etc., are selected from, by way of non-limiting example, at least one nucleotide (e.g., a polynucleotide), at least one carbohydrate or at least one amino acid (e.g., a peptide). In specific embodiments, the therapeutic agent is a polynucleotide, an oligonucleotide, a gene expression modulator, a knockdown agent, an siRNA, an RNAi agent, a dicer substrate, an miRNA, an shRNA, an antisense oligonucleotide, or an aptamer. In other specific embodiments, the therapeutic agent is an aiRNA (Asymmetric RNA duplexes mediate RNA interference in mammalian cells. Xiangao Sun, Harry A Rogoff, Chiang J Li Nature Biotechnology 26, 1379-1382 (2008)). In certain embodiments, the therapeutic agent is a protein, peptide, dominant-negative protein, enzyme, antibody, or antibody fragment. In some embodiments, the therapeutic agent is a carbohydrate, or a small molecule with a molecular weight of greater than about 500 Daltons.

In certain embodiments, one or more of the plurality of block copolymers is attached to a therapeutic agent.

In some embodiments, the shell of the micelle and/or hydrophilic block of one or more of the block copolymers comprises at least one nucleotide, at least one carbohydrate, or at least one amino acid. In certain embodiments, the shell of the micelle and/or hydrophilic block of one or more of the block copolymers comprises polynucleotide, an oligonucleotide, a gene expression modulator, a knockdown agent, an siRNA, an RNAi agent, a dicer substrate, an miRNA, an shRNA, an antisense oligonucleotide, an aptamer, a proteinaceous therapeutic agent, a protein, a peptide, an enzyme, a hormone, an antibody, an antibody fragment, a carbohydrate, a small molecule with a molecular weight of greater than about 500 Daltons, or a combination thereof.

In some embodiments, the micelles described herein comprise a polynucleotide, wherein the polynucleotide is a mammalian expression vector. In another embodiment, the micelles described herein comprise a polynucleotide that is designed to recombine with and correct an endogenous gene sequence in a human. In some embodiments, a polynucleotide provided in a hydrophilically-shielded micelle having membrane-destabilizing copolymers described herein is a gene expression modulator.

A mammalian expression vector comprises a complimentary DNA sequence (a “cDNA” or mini-gene) that is functionally linked to a promoter region such that the promoter drives expression of the cDNA. In certain instances, mammalian expression vectors also comprise a polyadenylation signal at the 3′ end of the cDNA. A promoter region is a nucleotide segment that is recognized by a RNA polymerase molecule, in order to initiate RNA synthesis (i.e., transcription), and may also include other transcriptional regulatory elements such as enhancers. Any number of transcriptional regulatory sequences may be used to mediate expression of linked genes in mammalian expression vectors. Promoters include but are not limited to retroviral promoters, other viral promoters such as those derived from HSV or CMV, and promoters from endogenous cellular genes. Mammalian expression vectors also typically have an origin of replication from E. Coli to enable propagation as plasmids in bacteria.

In certain instances, it is desirable to be able to introduce mammalian expression vectors into mammalian cells in culture or in vivo. In some embodiments, expression vectors are transfected into mammalian cells using the micelles provided herein.

As described herein, the micelles provided herein are used, in some embodiments, for delivery of polynucleotides into a cell or to an individual in need thereof. In certain embodiments, the micelle's polycationic blocks (e.g., the hydrophilic blocks of the block copolymers described herein) bind to the mammalian expression vector DNA and complexes the DNA with the micelle. In certain instances, polycations bind to and complex with mammalian expression vectors DNA. In some embodiments, a hydrophilically-shielded micelle having membrane-destabilizing copolymers comprising a polynucleotide complex is charge neutralized (e.g., the shell of the micelle or the hydrophilic block of a polymer of the micelle and the polynucleotide are substantially charge neutralized). Depending on the length of the polynucleotide, the length of the polycationic block is optionally adjusted to provide charge neutralization for the polynucleotide. In some instances, charge-neutralization is achieved by addition of cations and/or polycations into the formulation. In some embodiments, a hydrophilically-shielded micelle having membrane-destabilizing copolymers comprising a polymer and a polynucleotide (e.g., a 200+mer) is then diluted as necessary in an appropriate buffer and added directly to cells in culture. Expression of the transfected gene or cDNA in the resulting cells can be readily measured by including in the mammalian expression vector an expression cassette driving an indicator gene such as luciferase, chloramphenicol acetyl transferase or GFP. These genes are readily available and reporter assays are described.

In some embodiments, micelles provided herein are used for gene therapy. The treatment of diseases and disorders by gene therapy generally involves the transfer of new genetic information into cells. “Gene therapy vectors” comprise the new genetic material to be delivered, which is, optionally, in a mammalian expression vector. The uses of micelles include delivery of DNA sequences for gene replacement, inhibition of gene expression, gene correction or gene augmentation, or the introduction of genes to have some other desired effect, such as the modulation of immune responses. Inhibition of gene expression is accomplished in any suitable manner, including, by way of non-limiting example, by expression of gene cassettes in cells which express shRNAs or other RNAi agents.

In some embodiments, micelles having a polycationic hydrophilic block are mixed with gene therapy vectors, such that they become bound to the micelle. The micelle-gene therapy vector complex, in a suitable excipient (see below) is then administered to a living subject by routes including but not limited to intravenous, intra-arcticular, intrathecal, intracranial, inhalation, sub-cutaneous or intra-ocular.

In specific embodiments, a hydrophilically-shielded micelle having membrane-destabilizing copolymers provided herein comprises at least one polynucleotide (e.g., oligonucleotide). In some embodiments, the micelles provided herein are useful for delivering polynucleotides (e.g., oligonucleotides) to an individual in need thereof. In specific embodiments, the provided herein is a hydrophilically-shielded micelle having membrane-destabilizing copolymers that comprises at least 2, at least 4, at least 5, at least 10, at least 20, at least 30, at least 40, at least 50, at least 100 polynucleotides. In some embodiments, the micelle provided herein comprises 2-50 polynucleotides, 5-40 polynucleotides, 5-30 polynucleotides, 5-25 polynucleotides, 20-40 polynucleotides, or the like. In certain embodiments, the polynucleotide is an oligonucleotide gene expression modulator. In further embodiments, the polynucleotide is an oligonucleotide knockdown agent. In specific embodiments, the polynucleotide is an RNAi agent, dicer substrate, or siRNA. In certain embodiments, the micelle is a nanoparticle (e.g., a micelle) comprising a core, a shell and one or more polynucleotide, wherein the polynucleotide is not in the core of the micelle. In specific embodiments, the polynucleotide is incorporated into (e.g., is present in and/or forms a portion of) the shell of the micelle. In some embodiments, one or more polynucleotide (e.g., oligonucleotide or siRNA) is attached to hydrophilic block of the polymer (e.g., a block copolymer, or a non-membrane destabilizing diluent/carrier polymer) of the micelle. In various embodiments, attachment is achieved through one or more covalent bond, one or more non-covalent interaction, or a combination thereof. In some embodiments, the siRNA is covalently attached to a hydrophobic block of the block copolymer (e.g., a hydrophobic block). In specific embodiments, the siRNA is covalently attached to a hydrophobic block of the block copolymer and forms at least a portion of the shell of the micelle. In more specific embodiments, the siRNA is a hydrophilic block of the block copolymer. In other embodiments, the siRNA is attached to the hydrophilic block of a block copolymer, or to an optional polymer block (e.g., a spacer block). In some embodiments, one or more therapeutic agent (e.g., oligonucleotide or siRNA) is attached to a block copolymer provided herein in any manner suitable, e.g., by non-covalent association. Non-covalent association between (i) a polymer and/or an assembly of polymers provided herein (e.g., a micelle formed by a plurality of polymers) and (ii) one or more therapeutic agent (e.g., oligonucleotide) is achieved in any suitable manner, including, but not limited to, electrostatic interaction (including electrostatic interaction with a polymer having cationic groups and a therapeutic agent having anionic groups), hydrophobic interaction, affinity interaction, or a combination thereof. In certain embodiments, the one or more therapeutic agent and/or the polymers of the micelle is modified with chemical moieties that afford one or more therapeutic agent and/or polymers that have an affinity for one another, such as arylboronic acid-salicylhydroxamic acid, leucine zipper or other peptide motifs, ionic interactions between positive and negative charges on the micelle and therapeutic agent, or other types of non-covalent chemical affinity linkages. Additionally, in some embodiments, a double-stranded polynucleotide is associated with (e.g., complexed to) a polymer or micelle described herein. In some embodiments, a polymer or micelle is associated (e.g., complexed) with a nucleic acid minor groove binding agent or an intercalating agent that is attached (e.g., covalently) to a component (e.g., a polymer) of the micelle.

In some embodiments, the therapeutic agent (e.g., oligonucleotide) comprises at least one negative charge (e.g., comprises a negatively charged backbone) and is associated with a cationic shell of the micelle and/or a cationic hydrophilic block of a block copolymer of the micelle. In specific embodiments, the cationic shell or hydrophilic block at least partially neutralizes the negative charges present in the one or more therapeutic agents (e.g., oligonucleotides) attached to or present in the micelle. In certain embodiments, one or more therapeutic agent (e.g., one or more oligonucleotide, one or more siRNA, or a combination thereof) forms an association (e.g., a complex) with the polycationic hydrophilic blocks of the micelle. In some embodiments, the association (e.g., complex) between the micelle and therapeutic agent (e.g., oligonucleotide or siRNA) forms at any desired charge ratio of block copolymer forming the micelle to therapeutic agent (e.g., oligonucleotide or siRNA), e.g., between 1:1 and 16:1. In specific embodiments, the complex between the micelle and siRNA forms at the charge ratio of 2:1, 4:1 or 8:1. In other words, in some embodiments, the ratio of the number of cationic charges present in the shell of the micelle to the number of anionic charges present in the therapeutic agent is any desired value, e.g., about 1:1 to about 16:1, about 2:1 to about 8:1, about 4:1 to about 12:1, about 2:1, about 4:1, or about 8:1. In some embodiments, siRNA is charge-neutralized by a polycationic block of a block copolymer forming the micelle. For example, in some specific embodiments, a 20-base pair polynucleotide (e.g., oligonucleotide or siRNA) comprising 40 negative charges at physiologic pH is associated (e.g., complexed) with a micelle comprising a polyDMAEMA hydrophilic block (80 monomeric units in length, MW=11,680) with a pKa of about 7.4. At this pH, polyDMAEMA contains 40 negative charges, thereby resulting in a polynucleotide-hydrophilic block association (e.g., complex) that is substantially net neutral in charge. In certain instances, avoiding a large number of excess positive charges helps reduce in vitro and in vivo toxicity. In some embodiments, a therapeutic agent (e.g., oligonucleotide or siRNA) spontaneously associates with a positively charged shell of a hydrophilically-shielded micelle having membrane-destabilizing copolymers provided herein.

In some embodiments, a therapeutic agent (e.g., oligonucleotide or peptide) is chemically conjugated to the micelle and/or to one or more polymer of the micelle by any suitable chemical conjugation technique. Therapeutic agents are optionally conjugated to an end of the polymer, or to a pendant side chain of the polymer. In some embodiments, the therapeutic agent (e.g., a siRNA) is conjugated to pendant side chains on monomers present in the hydrophilic block of the polymer, including conjugated to a pendant side chain that also provides hydrophilic shielding. In some embodiments, micelles containing an RNAi agent are formed by conjugation of the RNAi agent with an already formed micelle comprising a plurality of polymers (e.g., block copolymers). In other embodiments, micelles containing an RNAi agent are formed by conjugation of the RNAi agent with a polymer (e.g., a block copolymer) and subsequently forming the micelle in any suitable manner, e.g., by self assembly of the resulting conjugates into a hydrophilically-shielded micelle having membrane-destabilizing copolymers comprising the RNAi agent. The covalent bond between a polymer and a therapeutic agent of a micelle described herein is, optionally, non-cleavable, or cleavable. In certain embodiments, a precursor of one or more RNAi agent (e.g. a dicer substrate) is attached to the micelle or to the polymeric units of micelle (e.g., the micelle by a non-cleavable bond). In some embodiments, one or more RNAi agent is attached through a cleavable bond. In certain embodiments, the cleavable bonds utilized in the micelles described herein include, by way of non-limiting example, disulfide bonds (e.g., disulfide bonds that dissociate in the reducing environment of the cytoplasm). In some embodiments, covalent association between a micelle (including the components thereof) and a therapeutic agent (e.g., an oligonucleotide or siRNA or peptide) is achieved through any suitable chemical conjugation method, including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In some embodiments, conjugation is also performed with pH-sensitive bonds and linkers, including, but not limited to, hydrazone and acetal linkages. Any other suitable conjugation method is optionally utilized as well, for example a large variety of conjugation chemistries are available (see, for example, Bioconjugation, Aslam and Dent, Eds, Macmillan, 1998 and chapters therein).

In some embodiments, the therapeutic agent is a proteinaceous agent. Conjugation of proteinatious therapeutic agents (e.g., a polypeptide) to the micelles provided herein is achieved according to a variety of conjugation processes by a chemical reaction involving one or more of the functional groups of the proteinaceous therapeutic agent (e.g., a polypeptide) with one or more of the functional groups present in the micelle (e.g., in the shell of the micelle or on a monomeric unit of the hydrophilic block). Polypeptide functional groups that are usually involved include but are not limited to amino, hydroxy, thiol, or carboxyl groups. Such groups can be present as a terminal group or present on the amino acid side chains. In some embodiments, the proteinaceous therapeutic agents are engineered to contain non-natural amino acids comprising special functional groups for formation of site-specific conjugates, e.g., azido groups for conjugation via “click” chemistry.

In certain embodiments, a conjugate of one or more therapeutic agent (e.g., oligonucleotide, such as an siRNA) with a polymer (e.g., block copolymer), wherein the polymer is a unimer or present in an assembled micelle, provided herein is prepared according to a process comprising the following two steps: (1) activating a modifiable end group (for example, 5′- or 3′-hydroxyl or) of an oligonucleotide using any suitable activation reagents, such as but not limited to 1-ethyl-3,3-dimethylaminopropyl carbodiimide (EDAC), imidazole, N-hydrosuccinimide (NHS) and dicyclohexylcarbodiimide (DCC), HOBt (1-hydroxybenzotriazole), p-nitrophenylchloroformate, carbonyldiimidazole (CDI), and N,N′-disuccinimidyl carbonate (DSC); and (2) covalently linking a block copolymer to the end of the oligonucleotide. In some embodiments, the 5′- or 3′-end modifiable group of an oligonucleotide is substituted by other functional groups prior to conjugation with the block copolymer. For example, hydroxyl group (—OH) is optionally substituted with a linker carrying sulfhydryl group (—SH), carboxyl group (—COOH), or amine group (—NH₂).

In yet another embodiment, an oligonucleotide comprising a functional group introduced into one or more of the bases (for example, a 5-aminoalkylpyrimidine), is conjugated to a polymer (e.g., block copolymer), wherein the polymer is a unimer or present in a micelle, provided herein using an activating agent or a reactive bifunctional linker according to any suitable procedure. A variety of such activating agents and bifunctional linkers is available commercially from such suppliers as Sigma, Pierce, Invitrogen and others.

In some embodiments, the micelle comprising an oligonucleotide or a plurality of oligonucleotides is formed by a spontaneous self assembly. Spontaneous self assembly of the micelle is achieved, in some embodiments, in a single pot. For example, in some embodiments, a micelle is self-assembled by diluting a solution of a polymer (e.g., block copolymer) described herein in an organic solvent (e.g., ethanol) with an aqueous media (e.g., water or PBS) is combined with one or more therapeutic agent (e.g., oligonucleotide or siRNA), the micelle comprising the polymers and one or more therapeutic agent spontaneously forming thereby. In other embodiments, spontaneous self assembly occurs by (1) contacting one or more therapeutic agent (e.g., oligonucleotide or siRNA) of interest with a polymer (e.g., membrane destabilizing block copolymer, a non-membrane destabilizing block copolymer, or a monoblock polymer) described herein so as to form a polymer-therapeutic agent conjugate; and (2) subjecting the polymer-therapeutic agent conjugates to conditions suitable to afford self assembly of the polymer-therapeutic agent conjugates into a micelle described herein. In some embodiments, the step of affording self assembly of the polymer-therapeutic agent conjugates further comprises contacting the polymer-therapeutic agent conjugates with an additional polymer (e.g., a non-conjugated block copolymer or monoblock polymer, or a diluent polymer, or the like, or a combination thereof).

### Targeting Moieties

In certain embodiments, micelles described herein comprise at least one targeting moiety (e.g., a moiety that targets a specific cell or type of cell). In some embodiments, the targeting moiety is in the core of the micelle, in the shell of the micelle, on the surface of the micelle, attached to a hydrophobic block of a block copolymer, attached to a hydrophilic block of a block copolymer, is a hydrophilic block of a membrane destabilizing agent, is present on a non-membrane destabilizing polymer within the micelle, is attached to a therapeutic agent within the micelle, attached to a pendant chain on a monomeric unit of a block copolymer, attached to the alpha or omega end of the block copolymer, or the like.

In specific instances, the micelles provided herein are useful for delivery of therapeutic agents to s cells of an individual. In certain instances, the efficiency of the cell uptake of the micelles is enhanced by incorporation of targeting moieties into or on the surface of the micelles. A “targeting moiety” (used interchangeably with “targeting agent”) recognizes a molecule on the surface of a cell (e.g., a select cell). In some embodiments, targeting moieties recognize a cell surface antigen or bind to a receptor on the surface of the target cell. Suitable targeting moieties include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting moieties of the micelles provided herein include, but are not limited, to the transferrin receptor type 1 and 2, the EGF receptor, HER2/Neu, VEGF receptors, integrins, NGF, CD2, CD3, CD4, CD8, CD19, CD20, CD22, CD33, CD43, CD38, CD56, CD69, and the asialoglycoprotein receptor.

Targeting moieties are attached, in various embodiments, to either end of a polymer (e.g., block copolymer) of the micelle, or to a side chain of a monomeric unit, or incorporated into a polymer block. Attachment of the targeting moiety to the polymer is achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In specific embodiments, “click” chemistry is used to attach the targeting ligand to the block copolymers forming the micelles provided herein (for example of “click” reactions, see Wu, P.; Fokin, V. V. Catalytic Azide-Alkyne Cycloaddition: Reactivity and Applications. Aldrichim. Acta 2007, 40, 7-17). A large variety of conjugation chemistries are optionally utilized (see, for example, Bioconjugation, Aslam and Dent, Eds, Macmillan, 1998 and chapters therein). In some embodiments, targeting ligands are attached to a monomer and the resulting compound is then used in the polymerization synthesis of a polymer (e.g., block copolymer) utilized in a micelle described herein. In some embodiments, targeting moieties are attached to a block of a first block copolymer, or to a block of a second block copolymer in a mixed micelle. In some embodiments, the targeting ligand is attached to the sense or antisense strand of siRNA bound to a polymer of the micelle. In certain embodiments, the targeting agent is attached to a 5′ or a 3′ end of the sense or the antisense strand.

In some embodiments, a block copolymer is synthesized by extension of the chain transfer agent (CTA) which comprises a targeting moiety, e.g., a galactose residue. In some instances, a targeting agent is attached to a group on a polymerizable monomer which is used to prepare the block copolymer provided herein.

In specific embodiments, the block copolymers forming the micelles provided herein are biocompatible. As used herein, “biocompatible” refers to a property of a polymer characterized by it, or its in vivo degradation products, being not, or at least minimally and/or reparably, injurious to living tissue; and/or not, or at least minimally and controllably, causing an immunological reaction in living tissue. With regard to salts, it is presently preferred that both the cationic and the anionic species be biocompatible. As used herein, “physiologically acceptable” is interchangeable with biocompatible. In some instances, the micelles and polymers used therein (e.g., block copolymers) exhibit low toxicity compared to cationic lipids.

### Cell Uptake

In some embodiments, the micelles comprising therapeutic agents (e.g., oligonucleotides or siRNA) are delivered to cells by endocytosis. Intracellular vesicles and endosomes are used interchangeably throughout this specification. Successful therapeutic agent (e.g., oligonucleotide or siRNA) delivery into the cytoplasm generally has a mechanism for endosomal escape. In certain instances, the micelles comprising therapeutic agents (e.g., oligonucleotide or siRNA) provided herein are sensitive to the lower pH in the endosomal compartment upon endocytosis. In certain instances, endocytosis triggers protonation or charge neutralization of anionically chargeable species (e.g., propyl acrylic acid units) of the micelles, resulting in a conformational transition in the micelles. In certain instances, this conformational transition results in a more hydrophobic membrane destabilizing form which mediates release of the therapeutic agent (e.g., oligonucleotide or siRNA) from the endosomes to the cytoplasm. In those micelles comprising siRNA, delivery of siRNA into the cytoplasm allows its mRNA knockdown effect to occur. In those micelles comprising other types of oligonucleotides, delivery into the cytoplasm allows their desired action to occur.

### EXAMPLES

Throughout the description of the present invention, various known acronyms and abbreviations are used to describe monomers or monomeric residues derived from polymerization of such monomers. Without limitation, unless otherwise noted: “BMA” (or the letter “B” as equivalent shorthand notation) represents butyl methacrylate or monomeric residue derived therefrom; “DMAEMA” (or the letter “D” as equivalent shorthand notation) represents N,N-dimethylaminoethyl methacrylate or monomeric residue derived therefrom; “Gal” refers to galactose or a galactose residue, optionally including hydroxyl-protecting moieties (e.g., acetyl) or to a pegylated derivative thereof (as described below); HPMA represents 2-hydroxypropyl methacrylate or monomeric residue derived therefrom; “MAA” represents methylacrylic acid or monomeric residue derived therefrom; “MAA(NHS)” represents N-hydroxyl-succinimide ester of methacrylic acid or monomeric residue derived therefrom; “PAA” (or the letter “P” as equivalent shorthand notation) represents 2-propylacrylic acid or monomeric residue derived therefrom, “PEGMA” refers to the pegylated methacrylic monomer, CH₃O(CH₂O)₇₋₈OC(O)C(CH₃)CH₂ or monomeric residue derived therefrom. In each case, any such designation indicates the monomer (including all salts, or ionic analogs thereof), or a monomeric residue derived from polymerization of the monomer (including all salts or ionic analogs thereof), and the specific indicated form is evident by context to a person of skill in the art.

### Example 1

### Preparation of Copolymers

Di-block polymers and copolymers of the following general formula are prepared:

[A1ₓ-/-A2_{y}]ₙ-[B1ₓ-/-B2_{y}-/-B3_{z}]₁₋₅ₙ

Where [A1-A2] is the first block copolymer, composed of residues of monomers A1 and A2
[B1-B2-B3] is the second block copolymer, composed of residues of monomers B1, B2, B3
x, y, z is the polymer composition in mole % monomer residue
n is molecular weight

Exemplary di-block copolymers:

[PEGMA_{w}]-[B-/-P-/-D]

[PEGMA_{w}-DMAEMA]-[B-/-P-/-D]

[PEGMA_{w}-MAA(NHS)]-[B-/-P-/-D]

Where:
B is butyl methacrylate
P is propyl acrylic acid
D is DMAEMA is dimethylaminoethyl methacrylate
PEGMA is polyethyleneglycol methacrylate where, for example, w=4-5 or 7-8 ethylene oxide units)
MAA(NHS) is methylacrylic acid-N-hydroxysuccinimide

### Example 1.1

### Synthesis of Block Copolymer Using Raft Polymerization

### A. Raft Chain Transfer Agent.

The synthesis of the chain transfer agent (CTA), 4-Cyano-4-(ethylsulfanylthiocarbonyl) sulfanylpentanoic acid (ECT), utilized for the following RAFT polymerizations, was adapted from a procedure by Moad et al., Polymer, 2005, 46(19): 8458-68. Briefly, ethane thiol (4.72 g, 76 mmol) was added over 10 minutes to a stirred suspension of sodium hydride (60% in oil) (3.15 g, 79 mmol) in diethyl ether (150 ml) at 0° C. The solution was then allowed to stir for 10 minutes prior to the addition of carbon disulfide (6.0 g, 79 mmol). Crude sodium S-ethyl trithiocarbonate (7.85 g, 0.049 mol) was collected by filtration, suspended in diethyl ether (100 mL), and reacted with Iodine (6.3 g, 0.025 mol). After 1 hour the solution was filtered, washed with aqueous sodium thiosulfate, and dried over sodium sulfate. The crude bis (ethylsulfanylthiocarbonyl) disulfide was then isolated by rotary evaporation. A solution of bis-(ethylsulfanylthiocarbonyl) disulfide (1.37 g, 0.005 mol) and 4,4′-azobis(4-cyanopentanoic acid) (2.10 g, 0.0075 mol) in ethyl acetate (50 mL) was heated at reflux for 18 h. Following rotary evaporation of the solvent, the crude 4-Cyano-4 (ethylsulfanylthiocarbonyl) sulfanylvpentanoic acid (ECT) was isolated by column chromatography using silica gel as the stationary phase and 50:50 ethyl acetate hexane as the eluent.

B. Poly(N,N-dimethylaminoethyl methacrylate) macro chain transfer agent (polyDMAEMA macroCTA).

The RAFT polymerization of DMAEMA was conducted in DMF at 30° C. under a nitrogen atmosphere for 18 hours using ECT and 2,2′-Azobis(4-methoxy-2,4-dimethyl valeronitrile) (V-70) (Wako chemicals) as the radical initiator. The initial monomer to CTA ratio ([CTA]₀/[M]₀ was such that the theoretical Mₙ at 100% conversion was 10,000 (g/mol). The initial CTA to initiator ratio ([CTA]ₒ/[I]ₒ) was 10 to 1. The resultant polyDMAEMA macro chain transfer agent was isolated by precipitation into 50:50 v:v diethyl ether/pentane. The resultant polymer was redissolved in acetone and subsequently precipitated into pentane (×3) and dried overnight in vacuo.

C. Block Copolymerization of DMAEMA, PAA, and BMA from a poly(DMAMEA) macroCTA.

The desired stoichiometric quantities of DMAEMA, PAA, and BMA were added to poly(DMAEMA) macroCTA dissolved in N,N-dimethylformamide (25 wt % monomer and macroCTA to solvent). For all polymerizations [M]ₒ/[CTA]ₒ and [CTA]ₒ/[I]ₒ were 250:1 and 10:1 respectively. Following the addition of V70 the solutions were purged with nitrogen for 30 min and allowed to react at 30° C. for 18 h. The resultant diblock copolymers were isolated by precipitation into 50:50 v:v diethyl ether/pentane. The precipitated polymers were then redissolved in acetone and subsequently precipitated into pentane (×3) and dried overnight in vacuo. Gel permeation chromatography (GPC) was used to determine molecular weights and polydispersities (PDI, M_{w}/Mₙ) of both the poly(DMAEMA) macroCTA and diblock copolymer samples in DMF with respect to polymethyl methacrylate standards (SEC Tosoh TSK-GEL R-3000 and R-4000 columns (Tosoh Bioscience, Montgomeryville, Pa.) connected in series to a Viscotek GPCmax VE2001 and refractometer VE3580 (Viscotek, Houston, Tex.). HPLC-grade DMF containing 1.0 wt % LiBr was used as the mobile phase.

### Example 1.2

### Preparation of Second Block (B1-B2-B3) Copolymerization of DMAEMA, PAA, and BMA from a poly(PEGMA) macroCTA

The desired stoichiometric quantities of DMAEMA, PAA, and BMA were added to poly(PEGMA) macroCTA dissolved in N,N-dimethylformamide (25 wt % monomer and macroCTA to solvent). For all polymerizations [M]ₒ/[CTA]ₒ and [CTA]ₒ/[I]ₒ were 250:1 and 10:1 respectively. Following the addition of AIBN the solutions were purged with nitrogen for 30 min and allowed to react at 68° C. for 6-12 h. The resulting diblock copolymers were isolated by precipitation into 50:50 v:v diethyl ether/pentane. The precipitated polymers were then redissolved in acetone and subsequently precipitated into pentane (×3) and dried overnight in vacuo. Gel permeation chromatography (GPC) was used to determine molecular weights and polydispersities (PDI, M_{W}/Mₙ) of both the poly(PEGMA) macroCTA and diblock copolymer samples in DMF using a Viscotek GPCmax VE2001 and refractometer VE3580 (Viscotek, Houston, Tex.). HPLC-grade DMF containing 1.0 wt % LiBr was used as the mobile phase. NMR spectroscopy in CDCl₃ was used to confirm the polymer structure and calculate the composition of the 2^{nd} block.

### Example 1.3

### Preparation and Characterization of PEGMA-DMAEMA Co-Polymers

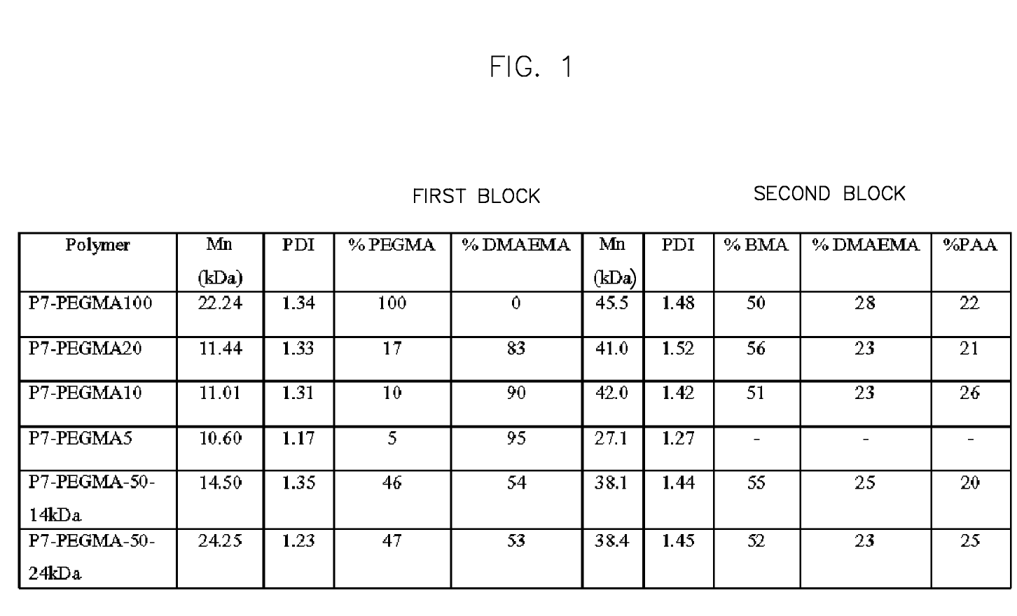

Polymer synthesis was carried out using a procedure similar to that described in Examples 1.1 and 1.2. The ratio of the PEGMA and DMAEMA in the first block was varied by using different feed ratios of the individual monomers to create the co-polymers described in FIG. 1.

### Example 1.4

### Preparation and Characterization of PEGMA-MAA(NHS) Co-Polymers

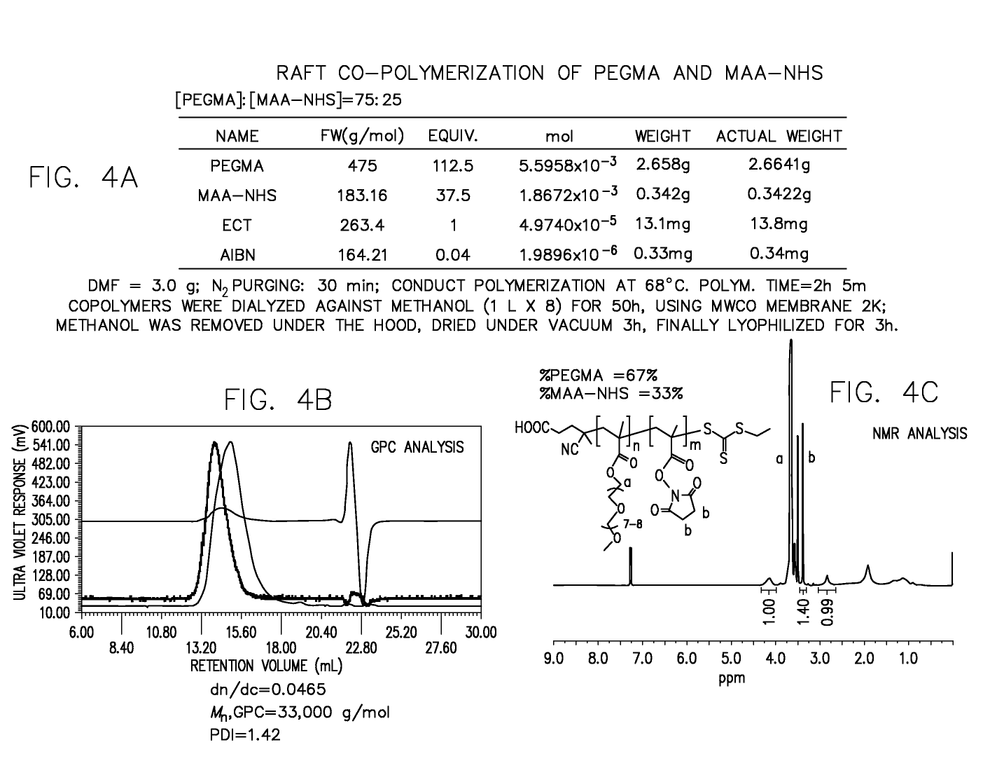

Polymer synthesis was performed as described in Examples 1.1 and 1.2, using monomer feed ratios to obtain the desired composition of the 1^{st} block copolymer. In some instances, [PEGMA_{w}-MAA(NHS)]-[B-P-D] polymer is prepared where the co-polymer ratio of monomers in the 1^{st} block is 75:25. NHS containing polymers can be incubated in aqueous buffer (phosphate or bicarbonate) at pH between 7.4 and 8.5 for 1-4 hrs at room temperature or 37° C. to generate the hydrolyzed (acidic) form. FIGS. 4A, 4B and 4C summarize the characterization of a PEGMA-MAA(NHS) co-polymer.

### Example 2

### Methods for Conjugating Targeting Ligands and Polynucleotides to a Copolymer

The following examples demonstrate methods for conjugating a targeting ligand (for example, galactose) or a polynucleotide therapeutic (for example siRNA) to a diblock copolymer. (1) The polymer is prepared using reversible addition fragmentation chain transfer (RAFT) (Chiefari et al. Macromolecules. 1998; 31(16):5559-5562) to form a galactose end-functionalized, diblock copolymer, using a chain transfer agent with galactose as the R-group substituent. (2) The first block of a diblock copolymer is prepared as a copolymer containing methylacrylic acid-N-hydroxysuccinimide (MAA(NHS)) where a galactose-PEG-amine is conjugated to the NHS groups or where an amino-disulfide siRNA is conjugated to the NHS, or where pyridyl disulfide amine is reacted with the NHS groups to form a pyridyl disulfide that is subsequently reacted with thiolated RNA to form a polymer-RNA conjugate.

### Example 2.1

### Preparation of galactose-PEG-amine and galactose-CTA

Scheme 1 illustrates the synthesis scheme for galactose-PEG-amine (compound 3) and the galactose-CTA (chain transfer agent) (compound 4).

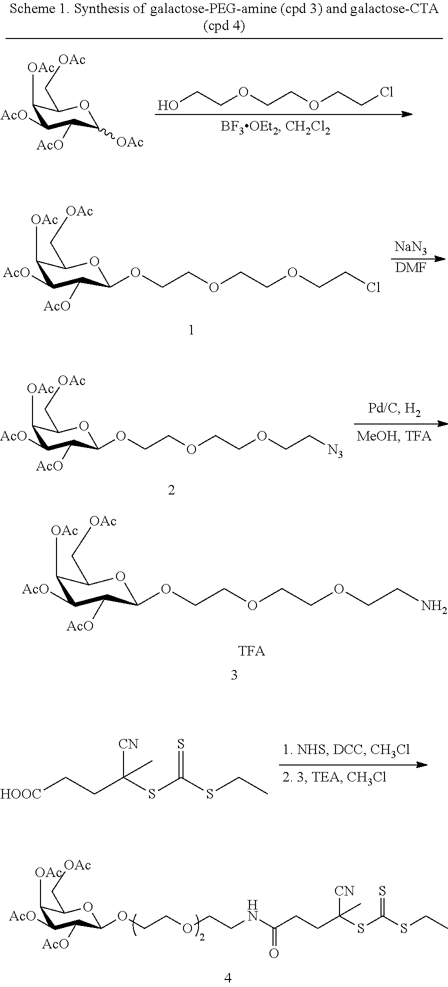

Compound 1: Pentaacetate galactose (10 g, 25.6 mmol) and 2-[2-(2-Chloroethoxy)ethoxy]ethanol (5.6 mL, 38.4 mmol) were dissolved in dry CH₂Cl₂ (64 mL) and the reaction mixture was stirred at RT for 1 h. The BF₃.OEt₂ (9.5 ml, 76.8 mmol) was added to the previous mixture dropwise over 1 h in an ice bath. The reaction mixture was stirred at room temperature (RT) for 48 h. After the reaction, 30 mL of CH₂Cl₂ was added to dilute the reaction. The organic layer was neutralized with saturated NaHCO_{3(aq)}, washed by brine and then dried by MgSO₄. The CH₂Cl₂ was removed under reduced pressure to get the crude product. The crude product was purified by flash column chromatography to get final product 1 as slight yellow oil. Yield: 55% TLC (I₂ and p-Anisaldhyde): EA/Hex: 1/1 (Rf: β=0.33; α=0.32; unreacted S.M 0.30).

Compound 2: Compound 1 (1.46 g, 2.9 mmol) was dissolved in dry DMF (35 mL) and the NaN₃ (1.5 g, 23.2 mmol) was added to the mixture at RT. The reaction mixture was heated to 85-90 C overnight. After the reaction, EA (15 mL) was added to the solution and water (50 mL) was used to wash the organic layer 5 times. The organic layer was dried by MgSO₄ and purified by flash column chromatography to get compound 2 as a colorless oil. Yield: 80%, TLC (I₂ and p-Anisaldhyde): EA/Hex: 1/1 (Rf: 0.33).

Compound 3: Compound 2 (1.034 g, 2.05 mmol) was dissolved in MeOH (24 mL) and bubbled with N₂ for 10 min and then Pd/C (10%) (90 mg) and TFA (80 uL) were added to the previous solution. The reaction mixture was bubbled again with H₂ for 30 min and then the reaction was stirred at RT under H₂ for another 3 h. The Pd/C was removed by celite and MeOH was evaporated to get the compound 3 as a sticky gel. Compound 3 can be used without further purification. Yield: 95%. TLC (p-Anisaldhyde): MeOH/CH₂Cl_{2 : 1/4} (Rf: 0.05).

Compound 4: ECT (0.5 g, 1.9 mmol), NHS (0.33 g, 2.85 mmol) and DCC (0.45 g, 2.19 mmol) were dissolved in CHCl₃ (15 mL) at 0 C. The reaction mixture was continuously stirred at RT overnight. Compound 3 (1.13 g, 1.9 mmol) and TEA (0.28 mL, 2.00 mmol) in CHCl₃ (10 mL) were added slowly to the previous reaction at 0 C. The reaction mixture was continuously stirred at RT overnight. The CH₃Cl was removed under reduced pressure and the crude product was purified by flash column chromatography to get the compound 4 as a yellow gel. Yield (35%). TLC: MeOH/CH₂Cl_{2: 1/9} (Rf: 0.75)

### Example 2.2

### Synthesis of [DMAEMA]-[BMA-PAA-DMAEMA]

A. Synthesis of DMAEMA macroCTA.

Polymerization: In a 20 mL glass vial (with a septa cap) was added 33.5 mg ECT (RAFT CTA), 2.1 mg AIBN (recrystallized twice from methanol), 3.0 g DMAEMA (Aldrich, 98%, was passed through a small alumina column just before use to remove the inhibitor) and 3.0 g DMF (high purity without inhibitor). The glass vial was closed with the Septa Cap and purged with dry nitrogen (carried out in an ice bath under stirring) for 30 min. The reaction vial was placed in a preheated reaction block at 70° C. The reaction mixture was stirred for 2 h 40 min. The septa cap was opened and the mixture was stirred in the vial in an ice bath for 2-3 minutes to stop the polymerization reaction.

Purification: 3 mL of acetone was added to the reaction mixture. In a 300 mL beaker was added 240 mL hexane and 60 mL ether (80/20 (v/v)) and under stirring the reaction mixture was added drop by drop to the beaker. Initially this produces an oil which is collected by spinning down the cloudy solution; yield=1.35 g (45%). Several precipitations were performed (e.g., 6 times) in hexane/ether (80/20 (v/v)) mixed solvents from acetone solution. Finally, the polymer was dried under vacuum for 8 h at RT; yield≈1 g. Summary: (M_{n,theory}=11,000 g/mol at 45% conv.)

| | FW | | | | | Actual | |
|---|---|---|---|---|---|---|---|
| Name | (g/mol) | Equiv. | mol | Weight | | weight | |
| DMAEMA | 157.21 | 150 | 0.0191 | 3.0 | g | 3.01 | g |
| ECT | 263.4 | 1 | 1.2722 × 10⁻⁴ | 33.5 | mg | 33.8 | mg |
| AIBN | 164.21 | 0.1 | 1.2722 × 10⁻⁵ | 2.1 | mg | 2.3 | mg |

DMF = 3.0 g;
N₂ Purging: 30 min;
Conduct polymerization at 70° C. for 2 h 45 min.

B. Synthesis of [BMA-PAA-DMAEMA] from DMAEMA macroCTA

All chemicals and reagents were purchased from Sigma-Aldrich Company unless specified. Butyl methacrylate (BMA) (99%), 2-(Dimethylamino) ethyl methacrylate (DMAEMA) (98%) were passed through a column of basic alumina (150 mesh) to remove the polymerization inhibitor. 2-propyl acrylic acid (PAA) (>99%) was purchased without inhibitor and used as received. Azobisisobutyronitrile (AIBN) (99%) was recrystallized from methanol and dried under vacuum. The DMAEMA macroCTA was synthesized and purified as described above (Mn˜10000; PDI˜1.3; >98%). N,N-Dimethylformamide (DMF) (99.99%) (Purchased from EMD) was reagent grade and used as received. Hexane, pentane and ether were purchased from EMD and they were used as received for polymer purification.

Polymerization: BMA (2.1 g, 14.7 mmoles), PAA (0.8389 g, 7.5 mmoles), DMAEMA (1.156 g, 7.35 mmoles), MacroCTA (0.8 g, 0.0816 mmoles), AIBN (1.34 mg, 0.00816 mmoles; CTA:AIBN 10:1) and DMF (5.34 ml) were added under nitrogen in a sealed vial. The CTA:Monomers ratio used was 1:360 (assuming 50% of conversion). The monomers concentration was 3 M. The mixture was then degassed by bubbling nitrogen into the mixture for 30 minutes and then placed in a heater block (Thermometer: 67° C.; display: 70-71; stiffing speed 300-400 rpm). The reaction was left for 6 hours, then stopped by placing the vial in ice and exposing the mixture to air.

Purification: Polymer purification was done from acetone/DMF 1:1 into hexane/ether 75/25 (three times). The resulting polymer was dried under vacuum for at least 18 hours. The NMR spectrum showed a high purity of the polymer. No vinyl groups were observed. The polymer was dialysed from ethanol against double de-ionized water for 4 days and then lyophilized. The polymer was analyzed by gel permeation chromatography (GPC) using the following conditions: Solvent: DMF/LiBr 1%. Flow rate: 0.75 ml/min. Injection volume: 100 μl.

Column temperature: 60° C. Poly (styrene) was used to calibrate the detectors. GPC analysis of the resulting Polymer: Mn=40889 g/mol. PDI=1.43. dn/dc=0.049967.

### Example 2.3

### Preparation and Characterization of [PEGMA-MAA(NHS)]-[B-P-D] and DMAEMA-MMA(NHS)-[B-P-D] Diblock Co-Polymers

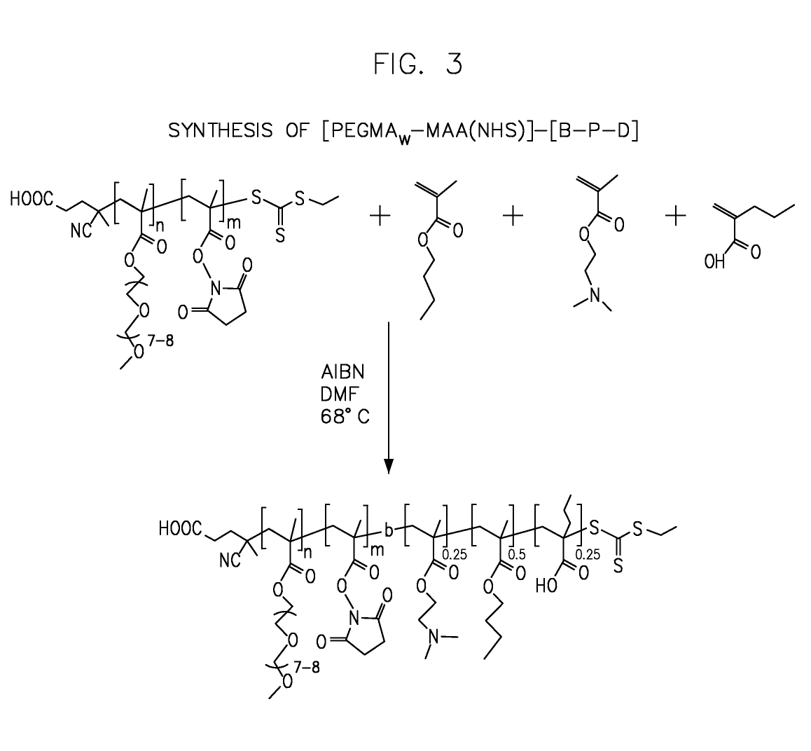

Polymer synthesis was performed as described in example 2.2 (and summarized in FIG. 3) using monomer feed ratios to obtain the desired composition of the 1^{st} block copolymer. FIG. 4 summarizes the synthesis and characterization of [PEGMA-MAA(NHS)]-[B-P-D] polymer where the co-polymer ratio of monomers in the 1^{st} block is 70:30.

### Example 2.4

### Conjugation of galactose-PEG-amine to PEGMA-MAA(NHS) to produce [PEGMA-MAA(Gal)]-[B-P-D] polymer

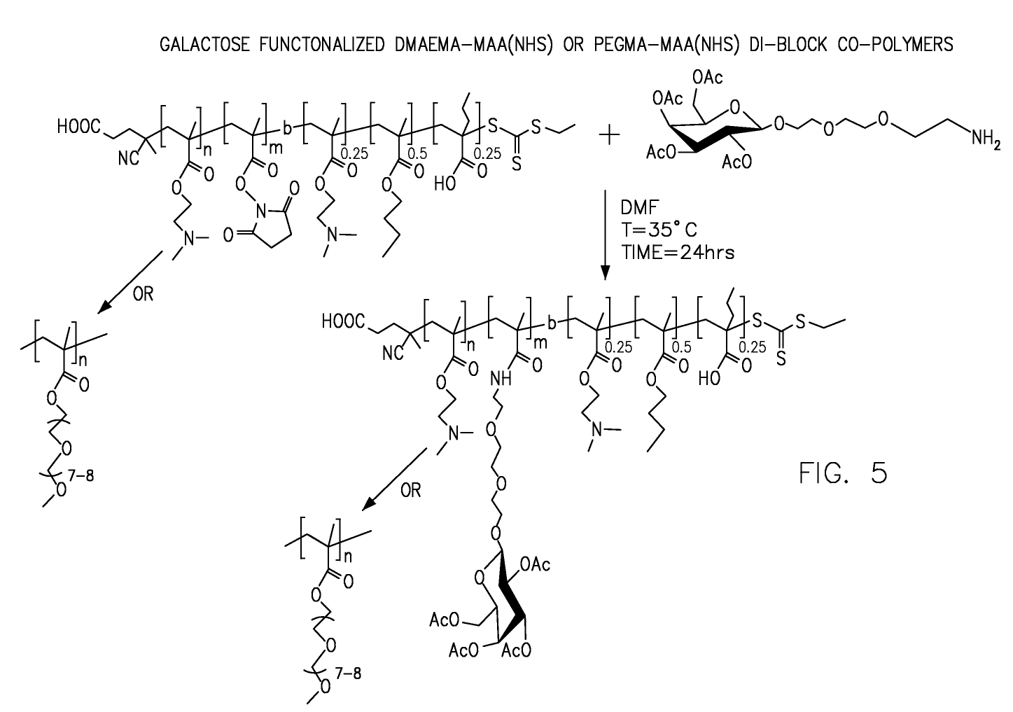

FIG. 5 illustrates the preparation of galactose functionalized DMAEMA-MAA(NHS) or PEGMA-MAA(NHS) di-block co-polymers. Polymer [DMAEMA-MAA(NHS)]-[B-P-D] or [PEGMA-MAA(NHS)]-[B-P-D] was dissolved in DMF at a concentration between 1 and 20 mg/ml. Galactose-PEG-amine prepared as described in example 2.1 (cpd 3) was neutralized with 1-2 equivalents of triethylamine and added to the reaction mixture at a ratio of 5 to 1 amine to polymer. The reaction was carried at 35° C. for 6-12 hrs, followed by addition of an equal volume of acetone, dialysis against deionized water for 1 day and lyophilization.

### Example 2.5

### Conjugation of siRNA to PEGMA-MAA(NHS)]-[B-P-D] to Produce [PEGMA-MAA(RNA)]-[B-P-D] Polymer

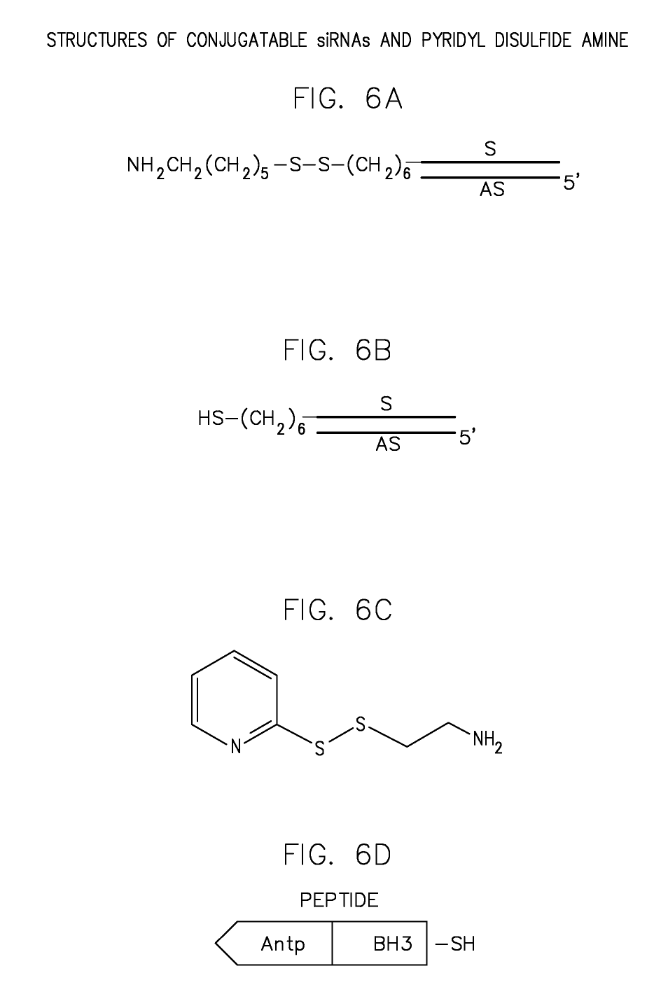

FIGS. 6 A and 6B shows the structures of 2 modified siRNAs that can be conjugated to NHS containing polymers prepared as described in example 2.3. siRNAs were obtained from Agilent (Boulder, Colo.). FIG. 6 C shows the structure of pyridyl disulfide amine used to derivatize NHS containing polymers to provide a disulfide reactive group for the conjugation of thiolated RNA (FIG. 6 B).

Reaction of NHS-containing polymer with amino-disulfide-siRNA. The reaction is carried out under standard conditions consisting of an organic solvent (for example, DMF or DMSO, or a mixed solvent DMSO/buffer pH 7.8.) at 35° C. for 4-8 hrs, followed by addition of an equal volume of acetone, dialysis against deionized water for 1 day and lyophilization.

Reaction of NHS-containing polymer with pyridyl-disulfide-amine and reaction with thiolated siRNA. Reaction of pyridyl disulfide amine with NHS containing polymers is carried out as described in example 2.4. Subsequently the lyophilized polymer is dissolved in ethanol at 50 mg/ml and diluted 10-fold in sodium bicarbonate buffer at pH 8. Thiolated siRNA (FIG. 6B) is reacted at a 2-5 molar excess over polymer NHS groups at 35° C. for 4-8 hrs, followed by dialysis against phosphate buffer, pH 7.4.

### Example 2.6

### Conjugation of a Therapeutic Peptide to a Pyridyl-Disulfide Modified Polymer

The pyridyl-disulfide modified polymer described in Example 2.5, PEGMA-MAA(NHS)]-[B-P-D], can also be used for conjugation to a therapeutic peptide (FIG. 6 D). The peptide is synthesized, prepared for conjugation, and the conjugation reaction carried out as described below to produce [PEGMA-MAA(Peptide)]-[B-P-D] polymer.

Fusion with the peptide transduction domain peptide transportin (also known as the Antennapedia peptide (Antp) sequence is utilized to synthesize a cell internalizing form of the Bak-BH3 peptide (Antp-BH3) containing a carboxy-terminal cysteine residue (NH₂-RQIKIWFQNRRMKWKKMGQVGRQLAIIGDDINRRYDSC-COOH). To ensure free thiols for conjugation, the peptide is reconstituted in water and treated for 1 hour with the disulfide reducing agent TCEP immobilized within an agarose gel. The reduced peptide (400 μM) is then reacted for 24 hours with the pyridyl disulfide end-functionalized polymer in phosphate buffer (pH 7) containing 5 mM ethylenediaminetetraacetic acid (EDTA).

Reaction of the pyridyl disulfide polymer end group with the peptide cysteine creates 2-pyridinethione, which can be spectrophotometrically measured to characterize conjugation efficiency. To further validate disulfide exchange, the conjugates are run on an SDS-PAGE 16.5% tricine gel. In parallel, aliquots of the conjugation reactions are treated with immobilized TCEP prior to SDS-PAGE to verify release of the peptide from the polymer in a reducing environment.

Conjugation reactions are conducted at polymer/peptide stoichiometries of 1, 2, and 5. UV spectrophotometric absorbance measurements at 343 nm for 2-pyridinethione release indicates conjugation efficiency. An SDS PAGE gel is utilized to further characterize peptide-polymer conjugates. At a polymer/peptide molar ratio of 1, a detectable quantity of the peptide forms dimers via disulfide bridging through the terminal cysteine. However, the thiol reaction to the pyridyl disulfide is favored, and the free peptide band is no longer visible at polymer/peptide ratios equal to or greater than 2. By treating the conjugates with the reducing agent TCEP, it is possible to cleave the polymer-peptide disulfide linkages as indicated by the appearance of the peptide band in these samples.

### Example 2.7

### Synthesis of gal-[DMAEMA]-[BMA-PAA-DMAEMA]

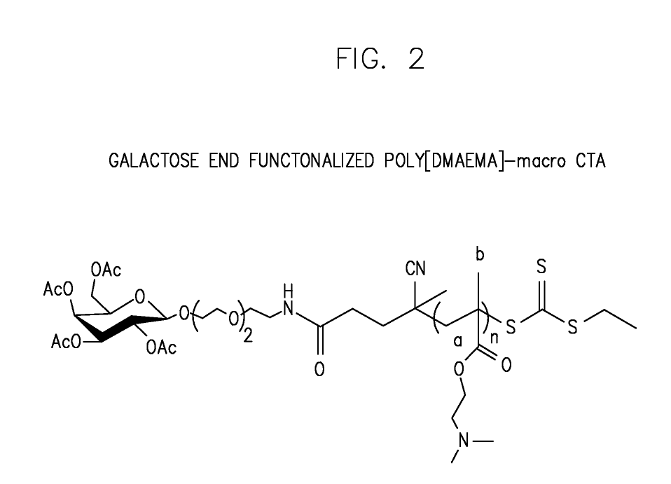

Synthesis was carried out as described in example 2.2. First, a galactose-DMAEMA macro-CTA was prepared (example 2.2.A.) except that galactose-CTA (example 2.1, cpd 4) was used in place of ECT as the chain transfer agent. This resulted in the synthesis of a polyDMAEMA with an end functionalized galactose (FIG. 2). The galactose-[DMAEMA]-macro-CTA was then used to synthesize the second block [BMA-PAA-DMAEMA] as described in example 2.2.B. Following synthesis, the acetyl protecting groups on the galactose were removed by incubation in 100 mM sodium bicarbonate buffer, pH 8.5 for 2 hrs, followed by dialysis and lyophilization. NMR spectroscopy was used to confirm the presence of the deprotected galactose on the polymer.

1. A composition comprising a micelle and a polynucleotide associated with the micelle, the micelle comprising a plurality of block copolymers, each including a hydrophilic block and a hydrophobic block;
the micelle being stable in an aqueous medium of about neutral pH;
the hydrophobic block comprising a pH dependent membrane destabilizing block;
the hydrophilic block of the copolymer comprising a plurality of constitutional units from a polymerizable monomer having a pendant group comprising a moiety of formula I 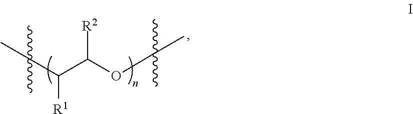
where
R¹ and R² are each independently selected from the group consisting of hydrogen, halogen, C₁-C₃ fluoroalkyl, and optionally substituted C₁-C₃ alkyl, and
n is an integer ranging from 2 to 20.

2. (canceled)

3. The composition of claim 1, wherein the pH dependent membrane destabilizing block comprises a plurality of pendant groups that are anionic at about neutral pH, and uncharged at about an endosomal pH.

4. The composition of claim 1, wherein the pH dependent membrane destabilizing block comprises a plurality of pendant groups that are cationic at about neutral pH, and cationic at about an endosomal pH.

5. (canceled)

6. The composition of claim 1, wherein the pH dependent membrane destabilizing block comprises a pendant group that is hydrophobic at about neutral pH and at about an endosomal pH.

7. The composition of claim 1, wherein the polynucleotide is not in the core of the micelle.

8-9. (canceled)

10. The composition of claim 1 wherein the micelle is covalently coupled to the polynucleotide.

11. A polymeric micelle, the micelle comprising
a block copolymer comprising a hydrophilic block and a hydrophobic block;
the micelle being stable in an aqueous medium of about neutral pH;
the hydrophobic block comprising a pH dependent membrane destabilizing block;
the hydrophilic block of the copolymer comprising a plurality of constitutional units from a polymerizable monomer having a pendant group comprising a moiety of formula I 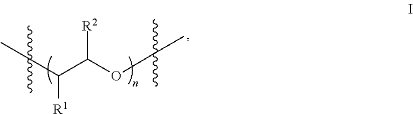
where R¹ and R² are each independently selected from the group consisting of hydrogen, halogen, C₁-C₃ fluoroalkyl, and optionally substituted C₁-C₃ alkyl, and
n is an integer ranging from 2 to 20.

12-14. (canceled)

15. The polymeric micelle of claim 1, wherein the pH dependent membrane destabilizing block comprises a plurality of pendant groups that are anionic at about neutral pH, and uncharged at about an endosomal pH.

16. The polymeric micelle of claim 15, wherein the pH dependent membrane destabilizing block comprises a plurality of pendant groups that are cationic at about neutral pH and cationic at about an endosomal pH.

17. The polymeric micelle of claim 15, wherein the pH dependent membrane destabilizing block comprises a plurality of pendant groups that are hydrophobic at about neutral pH and at about an endosomal pH.

18-19. (canceled)

20. A block copolymer comprising one or more hydrophilic blocks and one or more hydrophobic blocks,
the one or more hydrophilic blocks comprising
a plurality of constitutional units having a species charged or chargeable to a cation, and
a plurality of constitutional units from a polymerizable monomer having a pendant group comprising a moiety of formula I 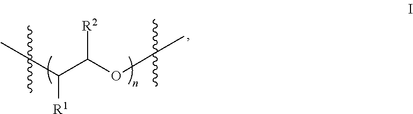
where R¹ and R² are each independently selected from the group consisting of hydrogen, halogen, C₁-C₃ fluoroalkyl, and optionally substituted C₁-C₃ alkyl, and
n is an integer ranging from 2 to 20, and
the one or more hydrophobic blocks comprises
a plurality of constitutional units having a species charged or chargeable to an anion, and
a plurality of constitutional units having a hydrophobic species.

21-24. (canceled)

25. The block copolymer of claim 20 wherein the hydrophobic block of the block copolymer further comprises a plurality of constitutional units having a species charged or chargeable to an anion, and a plurality of constitutional units having a hydrophobic species.

26. The block copolymer of claim 20 wherein the hydrophobic block of the block copolymer further comprises a plurality of constitutional units having a species charged or chargeable to an anion, a plurality of constitutional units having a species charged or chargeable to a cation, and a plurality of constitutional units having a hydrophobic species.

27-32. (canceled)

33. The block copolymer of claim 20 wherein the constitutional units are derived from a polymerizable monomer having a formula II 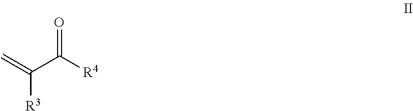
wherein
R³ is hydrogen, halogen, hydroxyl, or optionally substituted C₁-C₃ alkyl;
R⁴ is —SR⁵, —OR⁵, —NR⁶R⁷, or
R⁴ is a polyoxylated alkyl, optionally substituted by hydroxyl, thiol, —NR⁹R¹⁰, a cleavable moiety or a functionalizable moiety;
R⁵ is a polyoxylated alkyl, optionally substituted by hydroxyl, thiol, —NR⁹R¹⁰, a cleavable group or a functionalizable group;
R⁶ and R⁷ are each independently H or polyoxylated alkyl, optionally substituted by hydroxyl, thiol, —NR⁹R¹⁰, a cleavable group or a functionalizable group, provided that R⁶ and R⁷ are not both H; or
R⁶ and R⁷ together with the nitrogen to which they are attached form an optionally substituted heterocycle;
R⁹ and R¹⁰ are each independently H or C₁-C₆ alkyl; or
R⁹ and R¹⁰ together with the nitrogen to which they are attached form a heterocycle.

34. The block copolymer of claim 33, wherein R⁴ is an optionally substituted polyoxylated alkyl.

35. (canceled)

36. The block copolymer of claim 33 wherein the block copolymer comprises a plurality of constitutional units derived from a polymerizable monomer having a formula III 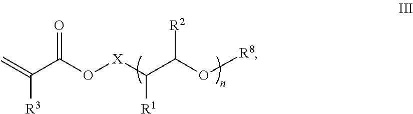
where
X is absent or optionally substituted C₁-C₃ alkyl;
R¹, R² and R³ are each independently hydrogen, halogen, C₁-C₃ fluoroalkyl or optionally substituted C₁-C₃ alkyl;
n is an integer ranging from 2 to 20,
R⁸ is hydrogen, C₁-C₆ alkyl, C₁-C₆ alkenyl, C₁-C₆ alkynyl, cycloalkyl, heterocycloalkyl, aryl, or heteroaryl optionally substituted by hydroxyl, thiol, —NR⁹R¹⁰, a cleavable group or a functionalizable group;
R⁹ and R¹⁰ are each independently H or C₁-C₆ alkyl; or
R⁹ and R¹⁰ together with the nitrogen to which they are attached form a heterocycle.

37. The block copolymer of claim 36, wherein R¹ and R² are each H.

38. The block copolymer of claim 37 wherein the block copolymer comprises a plurality of constitutional units derived from a polymerizable monomer having a formula IV 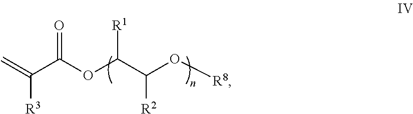
where
R¹, R² and R³ are each independently hydrogen, halogen, C₁-C₃ fluoroalkyl or optionally substituted C₁-C₃ alkyl;
n is an integer ranging from 2 to 20,
R⁸ is hydrogen, C₁-C₆ alkyl, C₁-C₆ alkenyl, C₁-C₆ alkynyl, cycloalkyl, heterocycloalkyl, aryl, or heteroaryl optionally substituted by hydroxyl, thiol, —NR⁹R¹⁰, a cleavable group or a functionalizable group;
R⁹ and R¹⁰ are each independently H or C₁-C₆ alkyl; or
R⁹ and R¹⁰ together with the nitrogen to which they are attached form a heterocycle.

39. The block copolymer of claim 38, wherein R¹ and R² are each H.

40-43. (canceled)

44. A method for intracellular delivery of a polynucleotide, comprising contacting a cell with the composition of claim 1.
